(19) United States
(12) Patent Application Publication
(10) Pub. No.: US 2021/0380680 A1
(43) Pub. Date: Dec. 9, 2021

(54) ANTI-CLAUDIN ANTIBODIES AND USES THEREOF

(71) Applicant: ZLlP HOLDING LIMITED, Grand Cayman (KY)

(72) Inventors: Yong LI, Shanghai (CN); Fengli SHAN, Shanghai (CN); Xu FANG, Shanghai (CN); Xinchuan DAI, Shanghai (CN); Shou LI, Shanghai (CN); Hong LI, Shanghai (CN); Yuan LIN, Shanghai (CN); Shali QI, Shanghai (CN); Yuejing JIANG, Shanghai (CN); Jing LI, Shanghai (CN); Bing WAN, Shanghai (CN); James YAN, Shanghai (CN); Yunpeng SU, Shanghai (CN); Valeria Rosa FANTIN, San Mateo, CA (US)

(21) Appl. No.: 17/311,307

(22) PCT Filed: Dec. 6, 2019
(86) PCT No.: PCT/CN2019/123588

§ 371 Date: Jun. 4, 2021

Related U.S. Application Data
(63) Continuation of application No. PCT/CN2018/119797, filed on Dec. 7, 2018.

(51) Int. Cl. C07K 16/28; A61P 35/00

(52) U.S. Cl. CPC C07K 16/28; A61P 35/00; A61K 2039/505; C07K 2317/734; C07K 2317/732

(57) ABSTRACT

Disclosed herein are anti-Claudin 18.2 antibodies and pharmaceutical compositions comprising the same. In some embodiments, also described herein are methods of treating a subject having a cancer with an anti-Claudin 18.2 antibody and methods of inducing cell kill effect with an anti-Claudin 18.2 antibody.

### CROSS-REFERENCE

This application claims priority to Patent Cooperation Treaty Application No. PCT/CN2018/119797, filed on Dec. 7, 2018, said application is incorporated herein by reference in its entirety for all purposes.

### BACKGROUND OF THE DISCLOSURE

Gastroesophageal and pancreatic cancers are among the malignancies with the highest unmet medical needs. Gastric cancer (GC) ranks as the third most common cause of cancer-related death and the largest proportion of gastric cancer patients is distributed in Eastern Asia, in particular in Korea, Mongolia, Japan, and China. Pancreatic cancer has the highest mortality rates of any cancer in the developed countries and is expected to increase in both the United States and China.

### SUMMARY OF THE DISCLOSURE

Disclosed herein, in certain embodiments, are anti-Claudin 18.2 antibodies and pharmaceutical compositions comprising the same. In certain embodiments, also described herein are methods of treating a subject having a cancer with an anti-Claudin 18.2 antibody and methods of inducing cell kill effect with an anti-Claudin 18.2 antibody.

Disclosed herein, in certain embodiments, is an anti-Claudin 18.2 (anti-CLDN18.2) antibody comprising a half maximal effective concentration (EC50) that is lower than an EC50 of reference antibody 175D10, wherein the reference antibody 175D10 comprises a heavy chain (HC) sequence set forth in SEQ ID NO: 98 and a light chain (LC) sequence set forth in SEQ ID NO: 99.

Disclosed herein, in certain embodiments, is an anti-Claudin 18.2 (anti-CLDN18.2) antibody comprising at least one mutation at a post-translational modification site.

Disclosed herein, in certain embodiments, is an anti-Claudin 18.2 (anti-CLDN18.2) antibody comprising at least one mutation at a Fc region that confer enhanced antibody-dependent cell-mediated cytotoxicity (ADCC), wherein the enhanced ADCC is compared to reference antibody 175D10 comprising a heavy chain (HC) sequence set forth in SEQ ID NO: 98 and a light chain (LC) sequence set forth in SEQ ID NO: 99. In some embodiments, the EC50 of the anti-CLDN18.2 antibody is about 5 nM or lower. In some embodiments, the EC50 of the anti-CLDN18.2 antibody is about 5 nM, about 4 nM, about 3 nM, about 2 nM, about 1 nM, about 0.5 nM, or lower.

Disclosed herein, in certain embodiments, is an anti-Claudin 18.2 (anti-CLDN18.2) antibody comprising a higher binding affinity to CLDN18.2 relative to a binding affinity of reference antibody 175D10, wherein the reference antibody 175D10 comprises a heavy chain (HC) sequence set forth in SEQ ID NO: 98 and a light chain (LC) sequence set forth in SEQ ID NO: 99.

In some embodiments, the anti-CLDN18.2 antibody comprises a variable heavy chain (VH) region and a variable light chain (VL) region, wherein the VH region comprises: CDR1 sequence GFSLTSYX₁VX₂; wherein X₁ is selected from N or G; and X₂ is selected from Y or H; CDR2 sequence VIWX₃X₄GX₅TX₆YX₇X₈X₉LX₁₀S; wherein X₃ is selected from N or P; X₄ is selected from T or G; X₅ is selected from A or N; X₆ is selected from R or N; X₇ is selected from N, Q, or E; X₈ is selected from S or I; X₉ is selected from T or A; and X₁₀ is selected from K or M; and CDR3 sequence DX₁₁X₁₂X₁₃X₁₄X₁₅X₁₆X₁₇X₁₈X₁₉X₂₀; wherein X₁₁ is selected from S or R; X₁₂ is selected from A or R; X₁₃ is selected from M or L; X₁₄ is selected from P or A; X₁₅ is selected from A or M; X₁₆ is selected from I or D; X₁₇ is selected from P or Y; X₁₈ is present or absence, if present, is F; X₁₉ is present or absence, if present, is A; and X₂₀ is present or absence, if present, is Y. In some embodiments, the VH region comprises CDR1 sequence X₂₁X₂₂X₂₃X₂₄X₂₅SFGMH; wherein X₂₁ is present or absence, if present, is G; X₂₂ is present or absence, if present, is F; X₂₃ is present or absence, if present, is T; X₂₄ is present or absence, if present, is F; and X₂₅ is present or absence, if present, is S; CDR2 sequence YISSGSX₂₆X₂₇IYYX₂₈DX₂₉X₃₀KG; wherein X₂₆ is selected from S or G; X₂₇ is selected from P or S; X₂₈ is selected from V or A; X₂₉ is selected from K or T; and X₃₀) is selected from L or V; and CDR3 sequence AX₃₁X₃₂X₃₃X₃₄X₃₅X₃₆X₃₇X₃₈X₃₉X₄₀X₄₁; wherein X₃₁ is selected from G or T; X₃₂ is selected from Y or S; X₃₃ is selected from A or Y; X₃₄ is selected from V or Y; X₃₅ is selected from R or Y; X₃₆ is selected from N or G; X₃₇ is selected from A or N; X₃₈ is selected from L or A; X₃₉ is selected from D or L; X₄₀ is selected from Y or E; and X₄₁ is present or absence, if present, is Y. In some embodiments, the VH region comprises CDR1 sequence consisting of SEQ ID NO: 1, CDR2 sequence VIWNTGATRYX₇SX₉LKS, and CDR3 sequence consisting of SEQ ID NO: 3, wherein X₇ is selected from N, Q, or E; and X₉ is selected from T or A. In some embodiments, the VH region comprises CDR1 sequence consisting of SEQ ID NO: 13, CDR2 sequence VIWPGGNTNYX₇X₈ALMS, and CDR3 sequence consisting of SEQ ID NO: 15, wherein X₇ is selected from N or E; and X₈ is selected from S or I. In some embodiments, the VH region comprises CDR1 sequence selected from SEQ ID NOs: 1, 7, 10, or 13; CDR2 sequence selected from SEQ ID NOs: 2, 4, 5, 6, 8, 11, 14, 16, or 17; and CDR3 sequence selected from SEQ ID NOs: 3, 9, 12, or 15. In some embodiments, the VH region comprises CDR1 sequence consisting of SEQ ID NO: 1; CDR2 sequence selected from SEQ ID NOs: 2, 4, 5, or 6; and CDR3 sequence consisting of SEQ ID NO: 3. In some embodiments, the VH region comprises CDR1 sequence consisting of SEQ ID NO: 13; CDR2 sequence selected from SEQ ID NOs: 14, 16, or 17; and CDR3 sequence consisting of SEQ ID NO: 15. In some embodiments, the VH region comprises CDR1 sequence consisting of SEQ ID NO: 7, CDR2 sequence consisting of SEQ ID NO: 8, and CDR3 sequence consisting of SEQ ID NO: 9. In some embodiments, the VH region comprises CDR1 sequence consisting of SEQ ID NO: 10, CDR2 sequence consisting of SEQ ID NO: 11, and CDR3 sequence consisting of SEQ ID NO: 12. In some embodiments, the VL region comprises CDR1 sequence selected from SEQ ID NOs: 18, 21, 24-28, 31-35, 38, or 39; CDR2 sequence selected from SEQ ID NOs: 19, 22, 29, or 36; and CDR3 sequence selected from SEQ ID NOs: 20, 23, 30, or 37. In some embodiments, the VL region comprises CDR1 sequence selected from SEQ ID NOs: 21 or 24-27; CDR2 sequence consisting of SEQ ID NO: 22; and CDR3 sequence consisting of SEQ ID NO: 23. In some embodiments, the VL region comprises CDR1 sequence selected from SEQ ID NOs: 28 or 31-34; CDR2 sequence consisting of SEQ ID NO: 29; and CDR3 sequence consisting of SEQ ID NO: 30. In some embodiments, the VL region comprises CDR1 sequence selected from SEQ ID NOs: 35, 38, or 39; CDR2 sequence consisting of SEQ ID NO: 36; and CDR3 sequence consisting of SEQ ID NO: 37. In some embodiments, the VL region comprises CDR1 sequence consisting of SEQ ID NO: 18, CDR2 sequence consisting of SEQ ID NO: 19, and CDR3 sequence consisting of SEQ ID NO: 20.

In some embodiments, the anti-CLDN18.2 antibody is a full-length antibody. In some embodiments, the anti-CLDN18.2 antibody is a binding fragment. In some embodiments, the anti-CLDN18.2 antibody comprises a monovalent Fab′, a divalent Fab2, a single-chain variable fragment (scFv), a diabody, a minibody, a nanobody, a single-domain antibody (sdAb), or a camelid antibody or binding fragment thereof. In some embodiments, the anti-CLDN18.2 antibody comprises a humanized antibody or binding fragment thereof, a chimeric antibody or binding fragment thereof, a monoclonal antibody or binding fragment thereof, or a bispecific antibody or binding fragment thereof.

In some embodiments, the anti-CLDN18.2 antibody comprises a mutation at a post-translational modification site. In some embodiments, the mutation is at an amino acid position 60, 61, or 62 of a VH region, and wherein the amino acid positions correspond to position 60, 61, or 62 of SEQ ID NO: 40. In some embodiments, the mutation is at an amino acid position 60 or 62 of SEQ ID NO: 40. In some embodiments, the mutation is at an amino acid position 60 or 61 of SEQ ID NO: 57. In some embodiments, the mutation at amino acid residue N60 is to glutamine or glutamic acid. In some embodiments, the mutation at amino acid residue S61 is to isoleucine. In some embodiments, the mutation at amino acid residue T62 is to alanine. In some embodiments, the mutation is at an amino acid position 31 or 32 of a VL region, and wherein the amino acid positions correspond to position 31 or 32 of SEQ ID NO: 46, 52, or 60. In some embodiments, the mutation is at amino acid position 31 or 32 of SEQ ID NO: 46, 52, or 60. In some embodiments, the mutation at amino acid residue N31 is to aspartic acid or glutamic acid. In some embodiments, the mutation at amino acid residue S32 is to leucine, valine, or isoleucine. In some embodiments, the mutation enhances the binding affinity of the modified anti-CLDN18.2 antibody relative to the reference antibody 175D10.

In some embodiments, the anti-CLDN18.2 antibody comprises a chimeric antibody or binding fragment thereof. In some embodiments, the chimeric antibody or binding fragment thereof comprises a VH region comprising at least 80%, 85%, 90%, 95%, or 100% sequence identity to SEQ ID NOs: 40-43 and a VL region comprising at least 80%, 85%, 90%, 95%, or 100% sequence identity to SEQ ID NO: 44. In some embodiments, the chimeric antibody or binding fragment thereof comprises a VH region comprising at least 80%, 85%, 90%, 95%, or 100% sequence identity to SEQ ID NO: 45 and a VL region comprising at least 80%, 85%, 90%, 95%, or 100% sequence identity to ID NOs: 46-50. In some embodiments, the chimeric antibody or binding fragment thereof comprises a VH region comprising at least 80%, 85%, 90%, 95%, or 100% sequence identity to SEQ ID NO: 51 and a VL region comprising at least 80%, 85%, 90%, 95%, or 100% sequence identity to SEQ ID NOs: 52-56. In some embodiments, the chimeric antibody or binding fragment thereof comprises a VH region comprising at least 80%, 85%, 90%, 95%, or 100% sequence identity to SEQ ID NOs: 57-59 and a VL region comprising at least 80%, 85%, 90%, 95%, or 100% sequence identity to SEQ ID NOs: 60-62. In some embodiments, the chimeric antibody or binding fragment thereof comprises a CH region comprising at least 80%, 85%, 90%, 95%, or 100% sequence identity to SEQ ID NO: 63 and a CL region comprising at least 80%, 85%, 90%, 95%, or 100% sequence identity to SEQ ID NO: 64.

In some embodiments, the anti-CLDN18.2 antibody comprises a humanized antibody or binding fragment thereof. In some embodiments, the humanized antibody or binding fragment thereof comprises a VH region comprising at least 80%, 85%, 90%, 95%, or 100% sequence identity to SEQ ID NOs: 65-68 and a VL region comprising at least 80%, 85%, 90%, 95%, or 100% sequence identity to SEQ ID NOs: 69-73. In some embodiments, the humanized antibody or binding fragment thereof comprises a VH region comprising at least 80%, 85%, 90%, 95%, or 100% sequence identity to SEQ ID NOs: 74-76 and a VL region comprising at least 80%, 85%, 90%, 95%, or 100% sequence identity to SEQ ID NOs: 77-80. In some embodiments, the humanized antibody or binding fragment thereof comprises a VH region comprising at least 80%, 85%, 90%, 95%, or 100% sequence identity to SEQ ID NOs: 81-84 and a VL region comprising at least 80%, 85%, 90%, 95%, or 100% sequence identity to SEQ ID NOs: 85-88. In some embodiments, the humanized antibody or binding fragment thereof comprises a VH region comprising at least 80%, 85%, 90%, 95%, or 100% sequence identity to SEQ ID NOs: 89-92 and a VL region comprising at least 80%, 85%, 90%, 95%, or 100% sequence identity to SEQ ID NOs: 93-97.

In some embodiments, the anti-CLDN18.2 antibody comprises an IgM framework.

In some embodiments, the anti-CLDN18.2 antibody comprises an IgG2 framework.

In some embodiments, the anti-CLDN18.2 antibody comprises an IgG1 framework.

In some embodiments, the anti-CLDN18.2 antibody comprises one or more mutations in the FC region. In some embodiments, the one or more mutations comprise a mutation at amino acid position S239, amino acid position I332, amino acid position F243, amino acid position R292, amino acid position Y300, amino acid position V305, amino acid position P396 or a combination thereof. In some embodiments, one or more mutations in the FC region confer enhanced ADCC to the reference antibody 175D10. In some embodiments, the anti-CLDN18.2 antibody has a complement-dependent cytotoxicity (CDC) activity compared to the reference antibody 175D10.

In some embodiments, the anti-CLDN18.2 antibody is further conjugated to a payload. In some embodiments, the payload is an auristatin or its derivative thereof. In some embodiments, the auristatin derivative is monomethyl auristatin E (MMAE). In some embodiments, the auristatin derivative is monomethyl auristatin F (MMAF).

In some embodiments, the drug-to-antibody ratio (DAR) is about 2, about 3, or about 4.

In some embodiments, the anti-CLDN18.2 antibody shares a binding epitope with the reference antibody 175D10.

In some embodiments, the anti-CLDN18.2 antibody has a cross-binding activity to mouse and cynomolgus CLDN18.2 protein.

Disclosed herein, in certain embodiments, is an anti-Claudin 18.2 (anti-CLDN18.2) antibody that specifically binds to an isoform of CLDN18.2. In some embodiments, the isoform of CLDN18.2 is an isoform expressed in cell line SNU620.

Disclosed herein, in certain embodiments, is a nucleic acid polymer encoding an anti-CLDN18.2 antibody described herein.

Disclosed herein, in certain embodiments, is a vector comprising a nucleic acid polymer encoding an anti-CLDN18.2 antibody described herein.

Disclosed herein, in certain embodiments, is a pharmaceutical composition comprising: an anti-CLDN18.2 antibody described herein; and a pharmaceutically acceptable excipient. In some embodiments, the pharmaceutical composition is formulated for systemic administration. In some embodiments, the pharmaceutical composition is formulated for parenteral administration.

Disclosed herein, in certain embodiments, is a method of treating a subject having a cancer that is characterized with an overexpression of CLDN18.2 protein, comprising: administering to the subject an anti-CLDN18.2 antibody described herein or a pharmaceutical composition described herein, thereby treating the cancer in the subject. In some embodiments, the cancer is a gastrointestinal cancer. In some embodiments, the gastrointestinal cancer is a gastric cancer. In some embodiments, the gastrointestinal cancer is a pancreatic cancer. In some embodiments, the gastrointestinal cancer is an esophageal cancer or cholangiocarcinoma. In some embodiments, the cancer is lung cancer or ovarian cancer. In some embodiments, the method further comprises administering to the subject an additional therapeutic agent. In some embodiments, the additional therapeutic agent comprises a chemotherapeutic agent, an immunotherapeutic agent, a targeted therapeutic agent, a hormone-based therapeutic agent, a stem-cell based therapeutic agent, or radiation. In some embodiments, the additional therapeutic agent and the anti-CLDN18.2 antibody are administered simultaneously. In some embodiments, the additional therapeutic agent and the anti-CLDN18.2 antibody are administered sequentially. In some embodiments, the additional therapeutic agent is administered prior to the anti-CLDN18.2 antibody. In some embodiments, the additional therapeutic agent is administered after the administration of the anti-CLDN18.2 antibody. In some embodiments, the additional therapeutic agent and the anti-CLDN18.2 antibody are formulated as separate dosage. In some embodiments, the subject is a human.

A Disclosed herein, in certain embodiments, is a method of inducing cell kill effect, comprising: contacting a plurality of cells with an anti-CLDN18.2 antibody comprising a payload for a time sufficient to internalize the anti-CLDN18.2 antibody and thereby to induce the cell kill effect. In some embodiments, the anti-CLDN18.2 antibody comprises an anti-CLDN18.2 antibody described herein. In some embodiments, the payload comprises a maytansinoid, an auristatin, a taxoid, a calicheamicins, a duocarmycin, an amatoxin, or a derivative thereof. In some embodiments, the payload comprises an auristatin or its derivative thereof. In some embodiments, the payload is monomethyl auristatin E (MMAE). In some embodiments, the payload is monomethyl auristatin F (MMAF). In some embodiments, the cell is a cancer cell. In some embodiments, the cell is from a gastrointestinal cancer. In some embodiments, the gastrointestinal cancer is a gastric cancer. In some embodiments, the gastrointestinal cancer is a pancreatic cancer. In some embodiments, the gastrointestinal cancer is an esophageal cancer or cholangiocarcinoma. In some embodiments, the cell is from a lung cancer or an ovarian cancer. In some embodiments, the method is an in vitro method. In some embodiments, the method is an in vivo method. In some embodiments, the subject is a human.

Disclosed herein, in certain embodiments, is a kit comprising an anti-CLDN18.2 antibody described herein, a vector described herein, or a pharmaceutical composition comprising an anti-CLDN18.2 antibody described herein.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates engineered expression of CLDN18.2 on HEK293 cells.

FIG. 2 illustrates human CLDN18.2 DNA sequence.

FIG. 3 illustrates CLDN18.2 ECL1 DNA.

FIG. 4A-FIG. 4C illustrate dose-dependent binding curves of purified anti-CLDN18.2 mouse-generated antibodies on CHO-CLDN18.2 cells. Antibodies showed highest (FIG. 4A), higher (FIG. 4B) and similar or weaker (FIG. 4C) maximal binding compared to that of 175D10.

FIG. 5A-FIG. 5B illustrate antibodies binding to gastric cancer cell line SNU601 (FIG. 5A) and SNU620 (FIG. 5B). The numbering “1”, “2”, “3”, and “4” indicate 282A12, 175D10, 10106, and isotype controls, respectively.

FIG. 6A-FIG. 6D illustrate chimeric 364D1A7 and 413H9F8 specifically binding to CHO-CLDN18.2 cell line. FIG. 6A and FIG. 6B illustrate the binding curves of chimeric 364D1A7 on CHO-CLDN18.1 and CHO-CLDN18.2 cell lines. FIG. 6C and FIG. 6D illustrate the binding curves of chimeric 413H9F8 on CHO-CLDN18.1 and CHO-CLDN18.2 cell lines. CHO-CLDN18.1 cell line was utilized for experiments shown in FIG. 6A and FIG. 6C and CHO-CLDN18.2 cell line was utilized for experiments shown in FIG. 6B and FIG. 6D. Chimeric 175D10 and paternal antibodies serve as controls.

FIG. 7A-FIG. 7D illustrate does-dependent binding of chimeric 282A12F3 variants to CHO-CLDN18.2 cell line. FIG. 7A and FIG. 7C show the binding curves of chimeric antibody (xi282A12F3) and chimeric antibodies with mutant PTM sites (282A12F3-VH-N60Q and 282A12F3-VH-N60E) on CHO-CLDN18.1 cell line. FIG. 7B and FIG. 7D show the binding curves of chimeric antibody (xi282A12F3) and chimeric antibodies with mutant PTM sites (282A12F3-VH-N60Q and 282A12F3-VH-N60E) on CHO-CLDN18.2 cell line.

FIG. 8A-FIG. 8D illustrate dose-dependent binding of chimeric 413H9F8 variants to CHO-CLDN18.2 cell lines. FIG. 8A and FIG. 8C show the binding curve of murine antibody (413H9F8), chimeric antibody (xi413H9F8), and chimeric antibodies with mutant PTM sites (413H9F8-VL-N31E, 413H9F8-VL-S32L, and 413H9F8-VL-S32V) on CHO-CLDN18.1 cell line. FIG. 8B and FIG. 8D show the binding curve of murine antibody (413H9F8), chimeric antibody (xi413H9F8), and chimeric antibodies with mutant PTM sites (413H9F8-VL-N31E, 413H9F8-VL-S32L, and 413H9F8-VL-S32V) on CHO-CLDN18.2 cell line.

FIG. 9A-FIG. 9D illustrate dose-dependent binding of chimeric 364D1A7 variants to CHO-CLDN18.2 cell lines. FIG. 9A and FIG. 9C show the binding curves of murine antibody (364D1A7), chimeric antibodies (xi364D1A7), and chimeric antibodies with mutant PTM sites (364D1A7-VL-N31E, 364D1A7-VL-S32L, and 364D1A7-VL-S32V) on CHO-CLDN18.1 cell line. FIG. 9B and FIG. 9D show the binding curves of murine antibody (364D1A7), chimeric antibodies (xi364D1A7), and chimeric antibodies with mutant PTM sites (364D1A7-VL-N31E, 364D1A7-VL-S32L, and 364D1A7-VL-S32V) on CHO-CLDN18.2 cell line.

FIG. 10A-FIG. 10B illustrate dose-dependent binding of chimeric 357B8F8 variants to CHO-CLDN18.2 cell line. FIG. 10A shows the binding curves of chimeric 357B8F8 antibodies with mutant PTM sites on CHO-CLDN18.1 cell line. FIG. 10B shows the binding curves of chimeric 357B8F8 antibodies with mutant PTM sites on CHO-CLDN18.2 cell line.

FIG. 11A-FIG. 11C illustrate binding of exemplary chimeric antibody variants on SNU620 cancer cell line. Binding curves of chimeric antibodies with mutant PTM sites on SNU620 gastric cancer cell line are as follow: FIG. 11A, 413H9F8; FIG. 11B, 264D1A7; and FIG. 11C, 357B8F8.

FIG. 12A-FIG. 12D illustrate competitive binding of chimeric antibodies to CHO-CLDN18.2 cell line. Binding of xi175D10 (FIG. 12A), 282A12F3 (T62A) (FIG. 12B), 413H9F8-VL-S32V (FIG. 12C), and 364D1A7-VL-S32V (FIG. 12D) on CHO-CLDN18.2 cells were monitored after incubation with exemplary concentrations of xi175D10, 282A12F3(T62A), 413H9F8-VL-S32V, 364D1A7-VL-S32V, or hIgG1.

FIG. 13A-FIG. 13E illustrate cross-species binding activity on different species of CLDN18.2 by exemplary antibodies. Binding affinities of hz282 (FIG. 13A), xi175D10 (FIG. 13B), 413H9F8-VL-S32V (FIG. 13C), 364D1A7-VL-S32V (FIG. 13D), and 357B8F8-VH-S61I-VL-S32I (FIG. 13E) were determined on CHO cells expressing human (closed square), mouse (closed circle), or cynomolgus (closed triangle) CLDN18.2. hIgG1 was set as negative control.

FIG. 14A-FIG. 14B illustrate CLDN18.2 specific ADCC activity induced by anti-CLDN18.2 antibodies and FcR-TANK (CD16A-15V) cells. ADCC activities of anti-CLDN18.2 antibody variants were determined in CHO-CLDN18.1 (FIG. 14A) and CHO-CLDN18.2 cell lines (FIG. 14B).

FIG. 15 illustrates ADCC activity of chimeric antibody variants on NCI-N87 cell line. ADCC activity was analyzed at effector (FcR-TANK (CD16A-15V)): target cell ratio of 2:1, and 16-hour incubation time. Data from duplicated wells.

FIG. 16 illustrates ADCC activity of chimeric antibody variants on NUGC4-18.2 cell line. ADCC activity was analyzed at effector (PBMC): target cell ratio of 40:1, and 5-hour incubation time. Data from one donor with duplicated well.

FIG. 17 illustrates CDC activity of chimeric antibody variants on CHO-18.2 cell line.

FIG. 18A-FIG. 18B illustrate humanized 282A12F3 (T62A) antibodies binding to CHO-CLDN18.2 FIG. 18A shows the binding curves of humanized 282A12F3 (T62A) antibodies on CHO-CLDN18.2 cells. FIG. 18B shows the binding curves of humanized 282A12F3 (T62A) antibodies on CHO-CLDN18.1 cells.

FIG. 19A-FIG. 19B illustrate humanized 282A12F3 (T62A) antibodies binding to SNU620 gastric cancer cells. FIG. 19A shows the binding curves of humanized 282A12F3 (T62A) antibodies hz282-1hz282-10 on SNU620 gastric cancer cells. FIG. 19B shows the binding curves of humanized 282A12F3 (T62A) antibodies hz282-11hz282-20 on SNU620 gastric cancer cells.

FIG. 20A-FIG. 20D illustrate binding affinities of humanized 413H9F8-VL-S32V (strategy 1) to CHO-CLDN18.2 cells. Full binding curves of humanized 413H9F8-VL-S32V antibodies are illustrated as follows: 413H9F8-cp1, 413H9F8-cp2, and 413H9F8-cp3 in FIG. 20A; 413H9F8-cp4, 413H9F8-cp5, and 413H9F8-cp6 in FIG. 20B; 413H9F8-cp7, 413H9F8-cp8, and 413H9F8-cp9 in FIG. 20C; and 413H9F8-cp10, 413H9F8-cp811, and 413H9F8-cp12 in FIG. 20D. The experiments were carried out in CHO-CLDN18.2 cells.

FIG. 21A-FIG. 21D illustrate binding affinities of humanized 413H9F8-VL-S32V in strategy 2 on CHO-CLDN18.2 cells. Full binding curves of humanized 413H9F8-VL-S32V antibodies are illustrated as follows: 413H9F8-H1L1, 413H9F8-H2L1, 413H9F8-H3L1, and 413H9F8-H4L1 in FIG. 21A; 413H9F8-H1L2, 413H9F8-H2L2, 413H9F8-H3L2, and 413H9F8-H4L2 in FIG. 21B; 413H9F8-H1L3, 413H9F8-H2L3, 413H9F8-H3L3, and 413H9F8-H4L3 in FIG. 21C; 413H9F8-H1L4, 413H9F8-H2L4, 413H9F8-H3L4, and 413H9F8-H4L4 in FIG. 21D. The experiments were carried out in CHO-CLDN18.2 cells.

FIG. 22A-FIG. 22E illustrate binding affinities of humanized 364D1A7-VL-S32V on CHO-CLDN18.2 cells. Full binding curves of humanized 364D1A7-VL-S32V antibodies are illustrated as follows: 364D1A7-H1L1, 364D1A7-H2L1, 364D1A7-H3L1, and 364D1A7-H41L in FIG. 22A; 364D1A7-H1L2, 364D1A7-H2L2, 364D1A7-H3L2, and 364D1A7-H4L2 in FIG. 22B; 364D1A7-H1L3, 364D1A7-H2L3, 364D1A7-H3L3, and 364D1A7-H4L3 in FIG. 22C; 364D1A7-H1L4, 364D1A7-H2L4, 364D1A7-H3L4, and 364D1A7-H4L4 in FIG. 22D; 364D1A7-H1L5, 364D1A7-H2L5, 364D1A7-H3L5, and 364D1A7-H4L5 in FIG. 22E. The experiments were carried out in CHO-CLDN18.2 cells.

FIG. 23A-FIG. 23C illustrate binding affinities of humanized 413H9F8-VL-32V and 364D1A7-VL-S32V antibodies on CHO-CLDN18.2 cells. FIG. 23A and FIG. 23B show full binding curves of humanized 413H9F8-VL-S32V antibodies on CHO-CLDN18.2 cells. FIG. 23C shows the full binding curve of humanized 364D1A7-VL-S32V antibodies on CHO-CLDN18.2 cells.

FIG. 24A-FIG. 24C illustrate ADCC activity of humanized antibody variants with cR-TANK (CD16A-15V) cells against NCI-N87-CLDN18.2 gastric cancer cell line. Humanized antibodies of 413H9F8 (FIG. 24A and FIG. 24B) and 364D1A7 (FIG. 24C) antibodies were analyzed for their capability to induce ADCC with FcR-TANK (CD16A-15V) cells against NCI-N87-CLDN18.2 cells at an effector: target cell ratio of 8:1. Mixed cells were cultured for 4 hours.

FIG. 25A-FIG. 25C illustrate ADCC activity of humanized antibody variants with human PBMC against NUGC4-CLDN18.2 gastric cancer cell line. Humanized antibodies of 413H9F8 (FIG. 25A and FIG. 25B) and 364D1A7 (FIG. 25C) antibodies were analyzed for their abilities to induce ADCC with human PBMCs against NUGC4-CLDN18.2 cells at an effector: target cell ratio of 40:1, cells were cultured for 5 hours. Data are from one donor with duplicated wells.

FIG. 26A-FIG. 26B illustrate CDC activities of humanized antibody variants on CHO-18.2 cell line. CDC activities of humanized 413H9F8-VL-S32V (FIG. 26A) and 364D1A7-VL-S32V (FIG. 26B) antibodies were determined with human serum against CHO-CLDN18.2 cell.

FIG. 27 illustrates an exemplary design structure for Mab-mc-vc-PAB-MMAE used in the study.

FIG. 28A-FIG. 28B illustrate CLDN18.2-specific ADCs inhibiting the viability of HEK293-CLDN18.2 cells. Viability of HEK293-CLDN18.2 (FIG. 28A) and HK293 (FIG. 28B) cells was determined after treatment with ADCs xi175D10-vcMMAE (DAR=4.02), 282A12F3(T62A)-vcMMAE (DAR=3.94) and hIgG1-vcMMAE (DAR=3.91) and naked antibodies xi175D10 282A12F3(T62A) and hIgG1 for 5 days. Viability was determined in HEK293 cell line expressing CLDN18.2.

FIG. 29A-FIG. 29B illustrate CLDN18.2-specific ADCs inhibiting the viability of NCI-N87-CLDN18.2 and NUGC4-CLDN18.2 cells. Viability of NCI-N87-CLDN18.2 (FIG. 29A) and NUGC4-CLDN18.2 (FIG. 29B) cells was determined after treatment with ADCs xi175D10-vcMMAE (DAR=4.02), 282A12F3 (T62A)-vcMMAE (DAR=3.94), and hIgG1-vcMMAE (DAR=3.91) for 5 days.

FIG. 30A-FIG. 30B illustrate CLDN18.2-specific ADCs inhibited viability of PANC-1-CLDN18.2 cell. FIG. 30A shows the ADCC efficacy of 282A12F3 (T62A) on PANC-1-CLDN18.2 cells. FIG. 30B shows the viability of PANC-1-CLDN18.2 cells after treated with CLDN18.2-specific ADCs, xi175D10-vcMMAE (DAR=4.02), 282A12F3 (T62A)-vcMMAE (DAR=3.94), and hIgG1-vcMMAE (DAR=3.91) for 5 days.

FIG. 31 illustrates ADCC activity of 413H9F8-cp2 variants with FcR-TANK (CD16A-15V) cells against CHO-CLDN18.2 cell line.

FIG. 32 illustrates ADCC activities of 413H9F8-cp2 and 413H9F8-H2L2 variants with human PBMCs against NUGC4-CLDN18.2 gastric cancer cell line.

FIG. 33A-33B illustrate internalization of anti-CLDN18.2 antibodies by NUGC4-CLDN18.2 cells (FIG. 33A) and NCI-N87-CLDN18.2 cells (FIG. 33B).

FIG. 34 illustrates efficacies of anti-CLDN18.2 antibodies in human gastric cancer GA0006 patient derived xenograft (PDX) model in nude mice.

FIG. 35A-35E illustrate efficacy of anti-CLDN18.2 antibodies in mouse xenograft models of pancreatic cancer in Nu/Nu mice.

FIG. 36 illustrates combinatorial efficacies of anti-CLD1N8.2 antibodies and chemotherapy in human gastric cancer GA0006 patient derived xenograft (PDX) model.

### DETAILED DESCRIPTION OF THE DISCLOSURE

Claudins (CLDNs) are central tight junction proteins that regulate epithelial-cell barrier function and polarity, thereby creating a boundary between the apical and basolateral plasma membrane domains. To date, 27 members of the CLDN family have been described with different organ-specific expression patterns. It has been shown that the expression levels of claudins are often abnormal in human neoplasias. One of the CLDN family members, CLDN-18 isoform 2 (CLDN18.2) is a selective gastric lineage antigen, and its expression in normal tissues is confined to differentiated epithelial cells of the gastric mucosa.

The CLDN18.2 protein is highly conserved in mouse, rat, rabbit, dog, monkey, and human and comprises four transmembrane domains and two extracellular domains. About 8 of the 51 amino acid residues within the first extracellular domain differ from lung-tissue specific CLDN-18 isoform 1 (CLDN18.1), and may serve as an epitope for monoclonal antibody binding.

Under a cancer setting, CLDN18.2 has been shown to be involved in tumor development and progression. Indeed, CLDN18.2 has been shown to be displayed on the surface of human gastric cancer cells and its metastases (Sahin, et al, “Claudin-18 splice variant 2 is a pan-cancer target suitable for therapeutic antibody development,” Clin Cancer Res 2008; 14:7624-34) and its ectopic activation was observed in pancreatic cancer (Woll, et al., “Claudin 18.2 is a target for IMAB362 antibody in pancreatic neoplasms,” Int J Cancer 2014; 134: 731-739; and Tanaka, et al., “Claudin-18 is an early-stage marker of pancreatic carcinogenesis,”J Histochem Cytochem 2011; 59: 942-952). Aberrant activation of CLDN18.2 was also observed in bile duct, esophageal, ovarian, and lung cancers, and was associated with poor overall survival and lymph node metastasis (Shinozaki, et al., “Claudin-18 in biliary neoplasms. Its significance in the classification of intrahepatic cholangiocarcinoma,” Virchows Arch 2011; 459: 73-80; and Micke, et al., “Aberrantly activated claudin 6 and 18.2 as potential therapy targets in non-small-cell lung cancer,” Int J CNCER 2014; 135: 2206-2214).

Disclosed herein, in certain embodiments, are anti-CLDN18.2 antibodies and uses thereof. In some instances, the anti-CLDN18.2 antibodies are chimeric antibodies. In other instances, the anti-CLDN18.2 antibodies are humanized antibodies. In additional instances, disclosed herein are treatment methods and methods of inducing cell kill effect that utilize an anti-CLDN18.2 antibody.

### Anti-Claudin 18.2 Antibodies

Disclosed herein, in certain embodiments, are anti-Claudin 18.2 (anti-CLDN18.2) antibodies. In some instances, an anti-CLDN18.2 antibody binds to an extracellular domain of CLDN18.2. In some cases, the anti-CLDN18.2 antibody binds to the first extracellular domain of CLDN18.2. In some cases, the anti-CLDN18.2 antibody binds to an eight residue region within the first extracellular domain of CLDN18.2, e.g., residues 32-41 of human CLDN18.2 (UniProtKB Identifier P56856-2). In some embodiments, also described herein are anti-CLDN18.2 antibodies that comprise one or more mutations at post-translational modification sites, with different functional properties than a reference anti-CLDN18.2 antibody, and/or with selectivity toward an isoform of CLDN18.2.

In some embodiments, an anti-CLDN18.2 antibody described herein comprises a half maximal effective concentration (EC50) that is lower than an EC50 of a reference anti-CLDN18.2 antibody. In some instances, the reference antibody is 175D10, which comprises a heavy chain (HC) sequence and a light chain (LC) sequence set forth in SEQ ID NO: 98 and SEQ ID NO: 99, respectively. In some cases, the EC50 of the anti-CLDN18.2 antibody is about 5 nM or lower. In some cases, the EC50 of the anti-CLDN18.2 antibody is about 4 nM, about 3 nM, about 2 nM, about 1 nM, about 0.5 nM, or lower.

In some embodiments, an anti-CLDN18.2 antibody described herein comprises a higher binding affinity to CLDN18.2 relative to a binding affinity of a reference anti-CLDN18.2 antibody. In some cases, the reference antibody is 175D10, which comprises a heavy chain sequence and a light chain sequence set forth in SEQ ID NO: 98 and SEQ ID NO: 99, respectively.

In some embodiments, an anti-CLDN18.2 antibody described herein has an enhanced antibody-dependent cell-mediated cytotoxicity (ADCC) compared to a reference anti-CLDN18.2 antibody. In some cases, the reference antibody is 175D10, which comprises a heavy chain sequence and a light chain sequence set forth in SEQ ID NO: 98 and SEQ ID NO: 99, respectively. In some cases, the anti-CLDN18.2 antibody further comprises a mutation at an Fc region that confers enhanced ADCC.

In some embodiments, an anti-CLDN18.2 antibody described herein comprises at least one mutation at a post-translational modification site. 100′741 In some embodiments, an anti-CLDN18.2 antibody described herein specifically binds to an isoform of CLDN18.2. In some cases, the isoform of CLDN18.2 is an isoform expressed in cell line SNU620.

In some embodiments, the anti-CLDN18.2 antibody comprises a variable heavy chain (VH) region and a variable light chain (VL) region, wherein the VH region comprises CDR1 sequence GFSLTSYX₁VX₂; CDR2 sequence VIWX₃X₄GX₅TX₆YX₇X₈X₉LX₁₀S; and CDR3 sequence DX₁₁X₁₂X₁₃X₁₄X₁₅X₁₆X₁₇X₁₈X₁₉X₂₀; wherein X₁ is selected from N or G; X₂ is selected from Y or H; X₃ is selected from N or P; X₄ is selected from T or G; X₅ is selected from A or N; X₆ is selected from R or N; X₇ is selected from N, Q, or E; X₈ is selected from S or I; X₉ is selected from T or A; X₁₀ is selected from K or M; X₁₁ is selected from S or R; X₁₂ is selected from A or R; X₁₃ is selected from M or L; X₁₄ is selected from P or A; X₁₅ is selected from A or M; X₁₆ is selected from I or D; X₁₇ is selected from P or Y; X₁₈ is present or absence, if present, is F; X₁₉ is present or absence, if present, is A; and X₂₀ is present or absence, if present, is Y.

In some instances, the VH region comprises CDR1 sequence X₂₁X₂₂X₂₃X₂₄X₂₅SFGMH; CDR2 sequence YISSGSX₂₆X₂₇IYYX₂₈DX₂₉X₃₀KG; and CDR3 sequence AX₃₁X₃₂X₃₃X₃₄X₃₅X₃₆X₃₇X₃₈X₃₉X₄₀X₄₁; wherein X₂₁ is present or absence, if present, is G; X₂₂ is present or absence, if present, is F; X₂₃ is present or absence, if present, is T; X₂₄ is present or absence, if present, is F; X₂₅ is present or absence, if present, is S; X₂₆ is selected from S or G; X₂₇ is selected from P or S; X₂₈ is selected from V or A; X₂₉ is selected from K or T; and X₃₀ is selected from L or V; X₃₁ is selected from G or T; X₃₂ is selected from Y or S; X₃₃ is selected from A or Y; X₃₄ is selected from V or Y; X₃₅ is selected from R or Y; X₃₆ is selected from N or G; X₃₇ is selected from A or N; X₃₈ is selected from L or A; X₃₉ is selected from D or L; X₄₀ is selected from Y or E; and X₄₁ is present or absence, if present, is Y.

In some embodiments, the VH region comprises CDR1, CDR2, and CDR3 sequences selected from Table 1.

| | | SEQ | | SEQ | | SEQ |
|---|---|---|---|---|---|---|
| | | ID | | ID | | ID |
| VH | CDR1 | NO: | CDR2 | NO: | CDR3 | NO: |
| 282A12F3- | GFSLTS | 1 | VIWNTG | 2 | DSAMPA | 3 |
| VH | YNVY | | ATRYN | | IPFAY | |
| (Parent) | | | STLKS | | | |
| 282A12F3- | GFSLTS | 1 | VIWNTG | 4 | DSAMPA | 3 |
| VH- | YNVY | | ATRYQ | | IPFAY | |
| N60Q | | | STLKS | | | |
| 282A12F3- | GFSLTS | 1 | VIWNTG | 5 | DSAMPA | 3 |
| VH- | YNVY | | ATRYE | | IPFAY | |
| N60E | | | STLKS | | | |
| 282A12F3- | GFSLTS | 1 | VIWNTG | 6 | DSAMPA | 3 |
| VH- | YNVY | | ATRYN | | IPFAY | |
| NSA | | | SALKS | | | |
| (T62A) | | | | | | |
| 413H9F8- | GFTFSS | 7 | YISSGS | 8 | AGYAVR | 9 |
| VH | FGMH | | SPIYYV | | NALDY | |
| (Parent) | | | DKLKG | | | |
| 364D1A7- | SFGMH | 10 | YISSGS | 11 | ATSYYY | 12 |
| VH | | | GSIYYA | | GNALEY | |
| (Parent) | | | DTVKG | | | |
| 357B8F8- | GFSLTS | 13 | VIWPGG | 14 | DRRLAM | 15 |
| VH | YGVH | | NTNYN | | DY | |
| (Parent) | | | SALMS | | | |
| 357B8F8- | GFSLTS | 13 | VIWPGG | 16 | DRRLAM | 15 |
| VH- | YGVH | | NTNYE | | DY | |
| N60E | | | SALMS | | | |
| 357B8F8- | GFSLTS | 13 | VIWPGG | 17 | DRRLAM | 15 |
| VH- | YGVH | | NTNYN | | DY | |
| S61I | | | IALMS | | | |

In some instances, the VH region comprises CDR1 sequence consisting of SEQ ID NO: 1, CDR2 sequence VIWNTGATRYX₇SX₉LKS, and CDR3 sequence consisting of SEQ ID NO: 3, wherein X₇ is selected from N, Q, or E; and X₉ is selected from T or A.

In some instances, the VH region comprises CDR1 sequence consisting of SEQ ID NO: 13, CDR2 sequence VIWPGGNTNYX₇X₈ALMS, and CDR3 sequence consisting of SEQ ID NO: 15, wherein X₇ is selected from N or E; and X₈ is selected from S or I.

In some instances, the VH region comprises CDR1 sequence selected from SEQ ID NOs: 1, 7, 10, or 13; CDR2 sequence selected from SEQ ID NOs: 2, 4, 5, 6, 8, 11, 14, 16, or 17; and CDR3 sequence selected from SEQ ID NOs: 3, 9, 12, or 15.

In some instances, the VH region comprises CDR1 sequence consisting of SEQ ID NO: 1; CDR2 sequence selected from SEQ ID NOs: 2, 4, 5, or 6; and CDR3 sequence consisting of SEQ ID NO: 3.

In some instances, the VH region comprises CDR1 sequence consisting of SEQ ID NO: 13; CDR2 sequence selected from SEQ ID NOs: 14, 16, or 17; and CDR3 sequence consisting of SEQ ID NO: 15.

In some instances, the VH region comprises CDR1 sequence consisting of SEQ ID NO: 7, CDR2 sequence consisting of SEQ ID NO: 8, and CDR3 sequence consisting of SEQ ID NO: 9.

In some instances, the VH region comprises CDR1 sequence consisting of SEQ ID NO: 10, CDR2 sequence consisting of SEQ ID NO: 11, and CDR3 sequence consisting of SEQ ID NO: 12.

In some embodiments, the VL region comprises CDR1, CDR2, and CDR3 sequences selected from Table 2.

| | | SEQ | | SEQ | | SEQ |
|---|---|---|---|---|---|---|
| | | ID | | ID | | ID |
| VL | CDR1 | NO: | CDR2 | NO: | CDR3 | NO: |
| 282A12_ | KSSQS | 18 | LASTRES | 19 | QQYYD | 20 |
| VL | LFGSVR | | | | IPWT | |
| | QKNYLA | | | | | |
| 413H9F8- | KSSQSL | 21 | GASTRES | 22 | QNDLF | 23 |
| VL | LNSGNQ | | | | YPLT | |
| (Parent) | KNYLA | | | | | |
| 413H9F8- | KSSQSL | 24 | GASTRES | 22 | QNDLF | 23 |
| VL- | LDSGNQ | | | | YPLT | |
| N31D | KNYLA | | | | | |
| 413H9F8- | KSSQSL | 25 | GASTRES | 22 | QNDLF | 23 |
| VL- | LESGNQ | | | | YPLT | |
| N31E | KNYLA | | | | | |
| 413H9F8- | KSSQSL | 26 | GASTRES | 22 | QNDLF | 23 |
| VL- | LNLGNQ | | | | YPLT | |
| S32L | KNYLA | | | | | |
| 413H9F8- | KSSQSL | 27 | GASTRES | 22 | QNDLF | 23 |
| VL- | LNVGNQ | | | | YPLT | |
| S32V | KNYLA | | | | | |
| 364D1A7- | KSSQSL | 28 | WASTRKS | 29 | QNVYS | 30 |
| VL | FNSGNQ | | | | YPLT | |
| (Parent) | KNYLT | | | | | |
| 364D1A7- | KSSQSL | 31 | WASTRKS | 29 | QNVYS | 30 |
| VL- | FDSGNQ | | | | YPLT | |
| N31D | KNYLT | | | | | |
| 364D1A7- | KSSQSL | 32 | WASTRKS | 29 | QNVYS | 30 |
| VL- | FESGNQ | | | | YPLT | |
| N31E | KNYLT | | | | | |
| 364D1A7- | KSSQSL | 33 | WASTRKS | 29 | QNVYS | 30 |
| VL- | FNLGNQ | | | | YPLT | |
| S32L | KNYLT | | | | | |
| 364D1A7- | KSSQSL | 34 | WASTRKS | 29 | QNVYS | 30 |
| VL- | FNVGNQ | | | | YPLT | |
| S32V | KNYLT | | | | | |
| 357B8F8- | KSSQSL | 35 | WASTRES | 36 | QNDYS | 37 |
| VL | LNSGNQ | | | | YPFT | |
| (Parent) | KNYLT | | | | | |
| 357B8F8- | KSSQSL | 38 | WASTRES | 36 | QNDYS | 37 |
| VL- | LESGNQ | | | | YPFT | |
| N31E | KNYLT | | | | | |
| 357B8F8- | KSSQSL | 39 | WASTRES | 36 | QNDYS | 37 |
| VL- | LNIGNQ | | | | YPFT | |
| S32I | KNYLT | | | | | |

In some instances, the VL region comprises CDR1 sequence selected from SEQ ID NOs: 18, 21, 24-28, 31-35, 38, or 39; CDR2 sequence selected from SEQ ID NOs: 19, 22, 29, or 36; and CDR3 sequence selected from SEQ ID NOs: 20, 23, 30, or 37.

In some instances, the VL region comprises CDR1 sequence selected from SEQ ID NOs: 21 or 24-27; CDR2 sequence consisting of SEQ ID NO: 22; and CDR3 sequence consisting of SEQ ID NO: 23.

In some instances, the VL region comprises CDR1 sequence selected from SEQ ID NOs: 28 or 31-34; CDR2 sequence consisting of SEQ ID NO: 29; and CDR3 sequence consisting of SEQ ID NO: 30.

In some instances, the VL region comprises CDR1 sequence selected from SEQ ID NOs: 35, 38, or 39; CDR2 sequence consisting of SEQ ID NO: 36; and CDR3 sequence consisting of SEQ ID NO: 37.

In some instances, the VL region comprises CDR1 sequence consisting of SEQ ID NO: 18, CDR2 sequence consisting of SEQ ID NO: 19, and CDR3 sequence consisting of SEQ ID NO: 20.

In some embodiments, the anti-CLDN18.2 antibody comprises a VH region comprising CDR1 sequence GFSLTSYX₁VX₂; CDR2 sequence VIWX₃X₄GX₅TX₆YX₇X₈X₉LX₁₀S; and CDR3 sequence DX₁₁X₁₂X₁₃X₁₄X₁₅X₁₆X₁₇X₁₈X₁₉X₂₀; wherein X₁ is selected from N or G; X₂ is selected from Y or H; X₃ is selected from N or P; X₄ is selected from T or G; X₅ is selected from A or N; X₆ is selected from R or N; X₇ is selected from N, Q, or E; X₈ is selected from S or I; X₉ is selected from T or A; X₁₀ is selected from K or M; X₁₁ is selected from S or R; X₁₂ is selected from A or R; X₁₃ is selected from M or L; X₁₄ is selected from P or A; X₁₅ is selected from A or M; X₁₆ is selected from I or D; X₁₇ is selected from P or Y; X₁₈ is present or absence, if present, is F; X₁₉ is present or absence, if present, is A; and X₂₀ is present or absence, if present, is Y; and a VL region comprising CDR1 sequence selected from SEQ ID NOs: 18, 35, 38, or 39; CDR2 sequence selected from SEQ ID NOs: 19 or 36; and CDR3 sequence selected from SEQ ID NOs: 20 or 37.

In some embodiments, the anti-CLDN18.2 antibody comprises a VH region comprising CDR1 sequence X₂₁X₂₂X₂₃X₂₄X₂₅SFGMH; CDR2 sequence YISSGSX₂₆X₂₇IYYX₂₈DX₂₉X₃₀KG; and CDR3 sequence AX₃₁X₃₂X₃₃X₃₄X₃₅X₃₆X₃₇X₃₈X₃₉X₄₀X₄₁; wherein X₂₁ is present or absence, if present, is G; X₂₂ is present or absence, if present, is F; X₂₃ is present or absence, if present, is T; X₂₄ is present or absence, if present, is F; X₂₅ is present or absence, if present, is S; X₂₆ is selected from S or G; X₂₇ is selected from P or S; X₂₈ is selected from V or A; X₂₉ is selected from K or T; and X₃₀ is selected from L or V; X₃₁ is selected from G or T; X₃₂ is selected from Y or S; X₃₃ is selected from A or Y; X₃₄ is selected from V or Y; X₃₅ is selected from R or Y; X₃₆ is selected from N or G; X₃₇ is selected from A or N; X₃₈ is selected from L or A; X₃₉ is selected from D or L; X₄₀ is selected from Y or E; and X₄₁ is present or absence, if present, is Y; and a VL region comprising CDR1 sequence selected from SEQ ID NOs: 21, 24-28, or 31-34; CDR2 sequence selected from SEQ ID NOs: 22 or 29; and CDR3 sequence selected from SEQ ID NOs: 23 or 30.

In some embodiments, the anti-CLDN18.2 antibody comprises a VH region comprising CDR1 sequence consisting of SEQ ID NO: 1, CDR2 sequence VIWNTGATRYX₇SX₉LKS, and CDR3 sequence consisting of SEQ ID NO: 3, wherein X₇ is selected from N, Q, or E; and X₉ is selected from T or A; and a VL region comprising CDR1 sequence consisting of SEQ ID NO: 18, CDR2 sequence consisting of SEQ ID NO: 19, and CDR3 sequence consisting of SEQ ID NO: 20.

In some instances, the anti-CLDN18.2 antibody comprises a VH region comprising CDR1 sequence consisting of SEQ ID NO: 13, CDR2 sequence VIWPGGNTNYX₇X₈ALMS, and CDR3 sequence consisting of SEQ ID NO: 15, wherein X₇ is selected from N or E; and X₈ is selected from S or I; and a VL region comprising CDR1 sequence selected from SEQ ID NOs: 35, 38, or 39; CDR2 sequence consisting of SEQ ID NO: 36; and CDR3 sequence consisting of SEQ ID NO: 37.

In some instances, the anti-CLDN18.2 antibody comprises a VH region comprising CDR1 sequence selected from SEQ ID NOs: 1, 7, 10, or 13; CDR2 sequence selected from SEQ ID NOs: 2, 4, 5, 6, 8, 11, 14, 16, or 17; and CDR3 sequence selected from SEQ ID NOs: 3, 9, 12, or 15; and a VL region comprising CDR1 sequence selected from SEQ ID NOs: 18, 21, 24-28, 31-35, 38, or 39; CDR2 sequence selected from SEQ ID NOs: 19, 22, 29, or 36; and CDR3 sequence selected from SEQ ID NOs: 20, 23, 30, or 37.

In some instances, the anti-CLDN18.2 antibody comprises a VH region comprising CDR1 sequence consisting of SEQ ID NO: 1; CDR2 sequence selected from SEQ ID NOs: 2, 4, 5, or 6; and CDR3 sequence consisting of SEQ ID NO: 3; and a VL region comprising CDR1 sequence consisting of SEQ ID NO: 18, CDR2 sequence consisting of SEQ ID NO: 19, and CDR3 sequence consisting of SEQ ID NO: 20.

In some instances, the anti-CLDN18.2 antibody comprises a VH region comprising CDR1 sequence consisting of SEQ ID NO: 13; CDR2 sequence selected from SEQ ID NOs: 14, 16, or 17; and CDR3 sequence consisting of SEQ ID NO: 15; and a VL region comprising CDR1 sequence selected from SEQ ID NOs: 35, 38, or 39; CDR2 sequence consisting of SEQ ID NO: 36; and CDR3 sequence consisting of SEQ ID NO: 37.

In some instances, the anti-CLDN18.2 antibody comprises a VH region comprising CDR1 sequence consisting of SEQ ID NO: 7, CDR2 sequence consisting of SEQ ID NO: 8, and CDR3 sequence consisting of SEQ ID NO: 9; and a VL region comprising CDR1 sequence selected from SEQ ID NOs: 21 or 24-27; CDR2 sequence consisting of SEQ ID NO: 22; and CDR3 sequence consisting of SEQ ID NO: 23.

In some instances, the anti-CLDN18.2 antibody comprises a VH region comprising CDR1 sequence consisting of SEQ ID NO: 10, CDR2 sequence consisting of SEQ ID NO: 11, and CDR3 sequence consisting of SEQ ID NO: 12; and a VL region comprising CDR1 sequence selected from SEQ ID NOs: 28 or 31-34; CDR2 sequence consisting of SEQ ID NO: 29; and CDR3 sequence consisting of SEQ ID NO: 30.

In some embodiments, an anti-CLDN18.2 antibody described herein is a full-length antibody or a binding fragment thereof. In some cases, the anti-CLDN18.2 antibody is a chimeric antibody or a binding fragment thereof. In other cases, the anti-CLDN18.2 antibody is a humanized antibody or a binding fragment thereof. In additional cases, the anti-CLDN18.2 antibody is a monoclonal antibody or a binding fragment thereof.

In some instances, the anti-CLDN18.2 antibody comprises a monovalent Fab′, a divalent Fab2, a single-chain variable fragment (scFv), a diabody, a minibody, a nanobody, a single-domain antibody (sdAb), or a camelid antibody or binding fragment thereof.

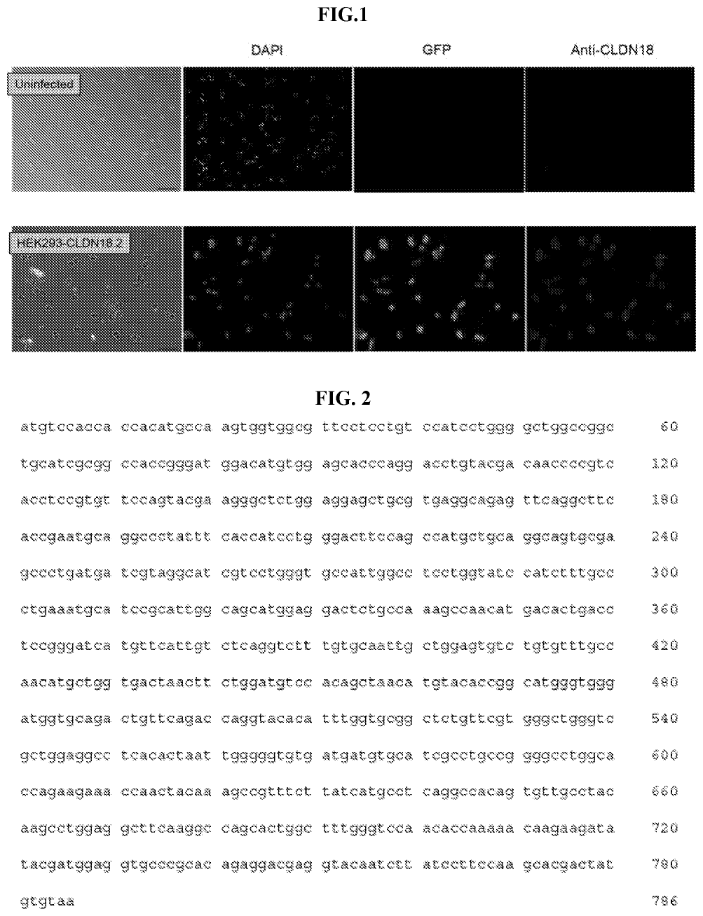

In some instances, the anti-CLDN18.2 antibody is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), nanobody, triplebody, tandems scFv (taFv), triple heads, tandem dAb/V1-1H, triple dAb/V1-1H, or tetravalent dAb/VHH. In some cases, the anti-CLDN18.2 antibody is a bispecific antibody or binding fragment thereof comprising a bispecific antibody format illustrated in FIG. 2 of Brinkmann and Kontermann, “The making of bispecific antibodies,” MABS 9(2): 182-212 (2017).

In some embodiments, an anti-CLDN18.2 antibody described herein comprises a mutation at a post-translational modification site. In some instances, the mutation is within the VH region. In other instances, the mutation is within the VL region. In additional instances, two or more mutations are within the VH region, the VL region, or a combination thereof.

In some instances, the mutation is at an amino acid position 60, 61, or 62 of the VH region of the anti-CLDN18.2 antibody, in which the amino acid position corresponds to position 60, 61, or 62 of SEQ ID NO: 40. In some instances, the mutation is at an amino acid position 60 or 61, which corresponds to position 60 or 61 of SEQ ID NO: 40. In some instances, the mutation is at an amino acid position 60 or 62, which corresponds to position 60 or 62 of SEQ ID NO: 40. In some cases, the mutation is at an amino acid position 60 (N60) or 61 (S61) of SEQ ID NO: 40. In some cases, the mutation is at an amino acid position 60 (N60) or 62 (T62) of SEQ ID NO: 40. In some cases, the mutation enhances the binding affinity of the anti-CLDN18.2 antibody relative to the reference antibody 175D10.

In some instances, the mutation is at an amino acid position 60, 61, or 62 of the VH region of the anti-CLDN18.2 antibody, in which the amino acid position corresponds to position 60, 61, or 62 of SEQ ID NO: 57. In some instances, the mutation is at an amino acid position 60 or 61, which corresponds to position 60 or 61 of SEQ ID NO: 57. In some instances, the mutation is at an amino acid position 60 or 62, which corresponds to position 60 or 62 of SEQ ID NO: 57. In some cases, the mutation is at an amino acid position 60 (N60) or 61 (S61) of SEQ ID NO: 57. In some cases, the mutation is at an amino acid position 60 (N60) or 62 (T62) of SEQ ID NO: 57. In some cases, the mutation enhances the binding affinity of the anti-CLDN18.2 antibody relative to the reference antibody 175D10.

In some instances, the amino acid residue N60 is mutated to a polar amino acid or an acidic amino acid. In some instances, the amino acid residue N60 is mutated to a polar amino acid selected from serine, threonine, asparagine, or glutamine. In some instances, the amino acid residue N60 is mutated to an acid amino acid selected from aspartic acid or glutamic acid. In some cases, the amino acid residue N60 is mutated to glutamine. In some cases, the amino acid residue N60 is mutated to glutamic acid.

In some instances, the amino acid residue S61 is mutated to a non-polar residue, optionally selected from alanine, cysteine, glycine, isoleucine, leucine, methionine, phenylalanine, proline, tryptophan, tyrosine, and valine. In some cases, the amino acid residue S61 is mutated to isoleucine.

In some instances, the amino acid residue T62 is mutated to a non-polar residue, optionally selected from alanine, cysteine, glycine, isoleucine, leucine, methionine, phenylalanine, proline, tryptophan, tyrosine, and valine. In some cases, the amino acid residue T62 is mutated to alanine.

In some instances, the mutation is at an amino acid position 31 or 32 of the VL region of the anti-CLDN18.2 antibody, in which the amino acid positions correspond to position 31 or 32 of SEQ ID NO: 46. In some cases, the mutation is at amino acid position 31 (N31) or 32 (S32) of SEQ ID NO: 46. In some cases, the mutation enhances the binding affinity of the anti-CLDN18.2 antibody relative to the reference antibody 175D10.

In some instances, the mutation is at an amino acid position 31 or 32 of the VL region of the anti-CLDN18.2 antibody, in which the amino acid positions correspond to position 31 or 32 of SEQ ID NO: 52. In some cases, the mutation is at amino acid position 31 (N31) or 32 (S32) of SEQ ID NO: 52. In some cases, the mutation enhances the binding affinity of the anti-CLDN18.2 antibody relative to the reference antibody 175D10.

In some instances, the mutation is at an amino acid position 31 or 32 of the VL region of the anti-CLDN18.2 antibody, in which the amino acid positions correspond to position 31 or 32 of SEQ ID NO: 60. In some cases, the mutation is at amino acid position 31 (N31) or 32 (S32) of SEQ ID NO: 60. In some cases, the mutation enhances the binding affinity of the anti-CLDN18.2 antibody relative to the reference antibody 175D10.

In some cases, the amino acid residue N31 is mutated to an acidic amino acid. In some cases, the amino acid residue N31 is mutated to aspartic acid or glutamic acid. In some cases, the amino acid residue N31 is mutated to aspartic acid. In some cases, the amino acid residue N31 is mutated to glutamic acid.

In some cases, the amino acid residue S32 is mutated to a non-polar residue, optionally selected from alanine, cysteine, glycine, isoleucine, leucine, methionine, phenylalanine, proline, tryptophan, tyrosine, and valine. In some cases, the amino acid residue S32 is mutated to leucine, valine, or isoleucine. In some cases, the amino acid residue S32 is mutated to leucine. In some cases, the amino acid residue S32 is mutated to valine. In some cases, the amino acid residue S32 is mutated to isoleucine.

In some embodiments, an anti-CLDN18.2 antibody described herein comprises a mutation at an amino acid position 60, 61, or 62 of the VH region of the anti-CLDN18.2 antibody, in which the amino acid position corresponds to position 60, 61, or 62 of SEQ ID NO: 57; and a mutation at an amino acid position 31 or 32 of the VL region of the anti-CLDN18.2 antibody, in which the amino acid positions correspond to position 31 or 32 of SEQ ID NO: 60. In some instances, the mutation is at an amino acid position 60 or 61, which corresponds to position 60 or 61 of SEQ ID NO: 57. In some instances, the mutation is at an amino acid position 60 or 62, which corresponds to position 60 or 62 of SEQ ID NO: 57. In some cases, the mutation is at an amino acid position 60 (N60) or 61 (S61) of SEQ ID NO: 57. In some cases, the mutation is at an amino acid position 60 (N60) or 62 (T62) of SEQ ID NO: 57. In some cases, the mutation is at amino acid position 31 (N31) or 32 (S32) of SEQ ID NO: 60. In some cases, the mutations enhance the binding affinity of the anti-CLDN18.2 antibody relative to the reference antibody 175D10.

In some embodiments, an anti-CLDN18.2 antibody described herein is a chimeric antibody or a binding fragment thereof. In some instances, the chimeric antibody or a binding fragment thereof comprises a VH region comprising at least 80%, 85%, 90%, 95%, or 100% sequence identity to SEQ ID NOs: 40-43 and a VL region comprising at least 80%, 85%, 90%, 95%, or 100% sequence identity to SEQ ID NO: 44. In some cases, the chimeric antibody or a binding fragment thereof comprises a VH region comprising at least 80%, 85%, 90%, 95%, or 100% sequence identity to SEQ ID NO: 45 and a VL region comprising at least 80%, 85%, 90%, 95%, or 100% sequence identity to ID NOs: 46-50. In some cases, the chimeric antibody or a binding fragment thereof comprises a VH region comprising at least 80%, 85%, 90%, 95%, or 100% sequence identity to SEQ ID NO: 51 and a VL region comprising at least 80%, 85%, 90%, 95%, or 100% sequence identity to SEQ ID NOs: 52-56. In some cases, the chimeric antibody or a binding fragment thereof comprises a VH region comprising at least 80%, 85%, 90%, 95%, or 100% sequence identity to SEQ ID NOs: 57-59 and a VL region comprising at least 80%, 85%, 90%, 95%, or 100% sequence identity to SEQ ID NOs: 60-62.

In some embodiments, the VH region and the VL region of a chimeric anti-CLDN18.2 antibody is illustrated in Table 3. The underlined regions denote the respective CDR1, CDR2, or CDR3 sequence.

| | | SEQ |
|---|---|---|
| | | ID |
| NAME | SEQUENCE | NO: |
| 282A12F3-VH | QVQLKESGPVLVQPSQTLSLTCTVAGFSLTSYNVYWVRQPPGKGLE | 40 |
| (Parent) | WMGVIWNTGATRYNSTLKSRLSISKDTSKSQVFLKMNSLQTEDTAT | |
| | YYCARDSAMPAIPFAYWGQGTLVTVSS | |
| 282A12F3-VH- | QVQLKESGPVLVQPSQTLSLTCTVAGFSLTSYNVYWVRQPPGKGLE | 41 |
| N60Q | WMGVIWNTGATRYQSTLKSRLSISKDTSKSQVFLKMNSLQTEDTAT | |
| | YYCARDSAMPAIPFAYWGQGTLVTVSS | |
| 282A12F3-VH- | QVQLKESGPVLVQPSQTLSLTCTVAGFSLTSYNVYWVRQPPGKGLE | 42 |
| N60E | WMGVIWNTGATRYESTLKSRLSISKDTSKSQVFLKMNSLQTEDTAT | |
| | YYCARDSAMPAIPFAYWGQGTLVTVSS | |
| 282A12F3-VH- | QVQLKESGPVLVQPSQTLSLTCTVAGFSLTSYNVYWVRQPPGKGLE | 43 |
| NSA | WMGVIWNTGATRYNSALKSRLSISKDTSKSQVFLKMNSLQTEDTAT | |
| (T62A) | YYCARDSAMPAIPFAYWGQGTLVTVSS | |
| 282A12_VL | DIVMTQSPSSLAVSAGETVTINCKSSQSLFGSVRQKNYLAWYQQKPG | 44 |
| | QSPKLLIYLASTRESGVPDRFIGSGSGTDFTLTISSVQAEDLAKYYCQ | |
| | QYYDIPWTFGGGTKLELK | |
| 413H9F8-VH | DVQLVESGGGSVQPGGSRRLSCAASGFTFSSFGMHWVRQAPEKGLE | 45 |
| (Parent) | WVAYISSGSSPIYYVDKLKGRFTVSRDNPKNTLFLQMTSLRSEDTAM | |
| | YYCARAGYAVRNALDYWGQGTSITVSS | |
| 413H9F8-VL | DIVMTQSPSSLSVSVGEKVTLSCKSSQSLLNSGNQKNYLAWYQQKT | 46 |
| (Parent) | GQPPKLLIYGASTRESGVPDRFIGSGSGTDFTLTISSVQAEDLAVYFC | |
| | QNDLFYPLTFGAGTKLELK | |
| 413H9F8-VL- | DIVMTQSPSSLSVSVGEKVTLSCKSSQSLLDSGNQKNYLAWYQQKT | 47 |
| N31D | GQPPKLLIYGASTRESGVPDRFIGSGSGTDFTLTISSVQAEDLAVYFC | |
| | QNDLFYPLTFGAGTKLELK | |
| 413H9F8-VL- | DIVMTQSPSSLSVSVGEKVTLSCKSSQSLLESGNQKNYLAWYQQKT | 48 |
| N31E | GQPPKLLIYGASTRESGVPDRFIGSGSGTDFTLTISSVQAEDLAVYFC | |
| | QNDLFYPLTFGAGTKLELK | |
| 413H9F8-VL- | DIVMTQSPSSLSVSVGEKVTLSCKSSQSLLNLGNQKNYLAWYQQKT | 49 |
| S32L | GQPPKLLIYGASTRESGVPDRFIGSGSGTDFTLTISSVQAEDLAVYFC | |
| | QNDLFYPLTFGAGTKLELK | |
| 413H9F8-VL- | DIVMTQSPSSLSVSVGEKVTLSCKSSQSLLNVGNQKNYLAWYQQKT | 50 |
| S32V | GQPPKLLIYGASTRESGVPDRFIGSGSGTDFTLTISSVQAEDLAVYFC | |
| | QNDLFYPLTFGAGTKLELK | |
| 364D1A7-VH | DVQLVESGGGLVQPGGSRKLSCAASGFTFSSFGMHWVRQAPEKGLE | 51 |
| (Parent) | WVAYISSGSGSIYYADTVKGRFTLSRDNPKNTLFLQMTSLRSEDTAI | |
| | YYCATSYYYGNALEYWGQGTSVTVSS | |
| 364D1A7-VL | DVVLTQSPSSLTVTEGEKVSMSCKSSQSLFNSGNQKNYLTWYQQKP | 52 |
| (Parent) | GQTPTLLIYWASTRKSGVPDRFTGSGSGTDFTLTINTVQAEDLAVYY | |
| | CQNVYSYPLTFGAGTKLDLK | |
| 364D1A7-VL- | DVVLTQSPSSLTVTEGEKVSMSCKSSQSLFDSGNQKNYLTWYQQKP | 53 |
| N31D | GQTPTLLIYWASTRKSGVPDRFTGSGSGTDFTLTINTVQAEDLAVYY | |
| | CQNVYSYPLTFGAGTKLDLK | |
| 364D1A7-VL- | DVVLTQSPSSLTVTEGEKVSMSCKSSQSLFESGNQKNYLTWYQQKP | 54 |
| N31E | GQTPTLLIYWASTRKSGVPDRFTGSGSGTDFTLTINTVQAEDLAVYY | |
| | CQNVYSYPLTFGAGTKLDLK | |
| 364D1A7-VL- | DVVLTQSPSSLTVTEGEKVSMSCKSSQSLFNLGNQKNYLTWYQQKP | 55 |
| S32L | GQTPTLLIYWASTRKSGVPDRFTGSGSGTDFTLTINTVQAEDLAVYY | |
| | CQNVYSYPLTFGAGTKLDLK | |
| 364D1A7-VL- | DVVLTQSPSSLTVTEGEKVSMSCKSSQSLFNVGNQKNYLTWYQQKP | 56 |
| S32V | GQTPTLLIYWASTRKSGVPDRFTGSGSGTDFTLTINTVQAEDLAVYY | |
| | CQNVYSYPLTFGAGTKLDLK | |
| 357B8F8-VH | QVQLKESGPGLVAPSQSLSITCTVSGFSLTSYGVHWVRQPPGKGLEW | 57 |
| (Parent) | LGVIWPGGNTNYNSALMSRLSISKDNSKSQVFLKMNSLQTDDTAMY | |
| | YCARDRRLAMDYWGQGTSVTVSS | |
| 357B8F8-VH- | QVQLKESGPGLVAPSQSLSITCTVSGFSLTSYGVHWVRQPPGKGLEW | 58 |
| N60E | LGVIWPGGNTNYESALMSRLSISKDNSKSQVFLKMNSLQTDDTAMY | |
| | YCARDRRLAMDYWGQGTSVTVSS | |
| 357B8F8-VH- | QVQLKESGPGLVAPSQSLSITCTVSGFSLTSYGVHWVRQPPGKGLEW | 59 |
| S61I | LGVIWPGGNTNYNIALMSRLSISKDNSKSQVFLKMNSLQTDDTAMY | |
| | YCARDRRLAMDYWGQGTSVTVSS | |
| 357B8F8-VL | DIVMTQSPSSLWTAGEKVTMTCKSSQSLLNSGNQKNYLTWYQQKP | 60 |
| (Parent) | GQPPKLLIYWASTRESGVPDRFTGSGSGTDFTLTISILQAEDLAVYYC | |
| | QNDYSYPFTFGSGTKLEIK | |
| 357B8F8-VL- | DIVMTQSPSSLTVTAGEKVTMTCKSSQSLLESGNQKNYLTWYQQKP | 61 |
| N31E | GQPPKLLIYWASTRESGVPDRFTGSGSGTDFTLTISILQAEDLAVYYC | |
| | QNDYSYPFTFGSGTKLFIK | |
| 357B8F8-VL- | DIVMTQSPSSLWTAGEKVTMTCKSSQSLLNIGNQKNYLTWYQQKP | 62 |
| S32I | GQPPKLLIYWASTRESGVPDRFTGSGSGTDFTLTISILQAEDLAVYYC | |
| | QNDYSYPFTFGSGTKLEIK | |

In some cases, the chimeric antibody or a binding fragment thereof further comprises a CH region comprising at least 80%, 85%, 90%, 95%, or 100% sequence identity to SEQ ID NO: 63 and a CL region comprising at least 80%, 85%, 90%, 95%, or 100% sequence identity to SEQ ID NO: 64. In some cases, the chimeric antibody or a binding fragment thereof comprises a CH region and a CL region as set forth in Table 4.

| | | SEQ |
|---|---|---|
| | | ID |
| | SEQUENCE | NO: |
| CH | ASTKGPSVFPLAPSSKSTSG | 63 |
| amino | GTAALGCLVKDYFPEPVTVS | |
| acid | WNSGALTSGVHTFPAVLQSS | |
| sequence | GLYSLSSVVTVPSSSLGTQT | |
| of | YICNVNHKPSNTKVDKKVEP | |
| human | KSCDKTHTCPPCPAPELLGG | |
| IgG1 | PSVFLFPPKPKDTLMISRTP | |
| | EVTCVVVDVSHEDPEVKFNW | |
| | YVDGVEVHNAKTKPREEQYN | |
| | STYRVVSVLTVLHQDWLNGK | |
| | EYKCKVSNKALPAPIEKTIS | |
| | KAKGQPREPQVYTLPPSREE | |
| | MTKNQVSLTCLVKGFYPSDI | |
| | AVEWESNGQPENNYKTTPPV | |
| | LDSDGSFFLYSKLTVDKSRW | |
| | QQGNVFSCSVMHEALHNHYT | |
| | QKSLSLSPGK | |
| CL amino | RTVAAPSVFIFPPSDEQLKS | 64 |
| acid | GTASVVCLLNNFYPREAKVQ | |
| sequence | WKVDNALQSGNSQESVTEQD | |
| of | SKDSTYSLSSTLTLSKADYE | |
| human | KHKVYACEVTHQGLSSPVTK | |
| IgG1 | SFNRGEC | |

In some embodiments, an anti-CLDN18.2 antibody described herein is a humanized antibody or a binding fragment thereof. In some instances, the humanized antibody or binding fragment thereof comprises a VH region comprising at least 80%, 85%, 90%, 95%, or 100% sequence identity to SEQ ID NOs: 65-68 and a VL region comprising at least 80%, 85%, 90%, 95%, or 100% sequence identity to SEQ ID NOs: 69-73. In some instances, the humanized antibody or binding fragment thereof comprises a VH region comprising at least 80%, 85%, 90%, 95%, or 100% sequence identity to SEQ ID NOs: 74-76 and a VL region comprising at least 80%, 85%, 90%, 95%, or 100% sequence identity to SEQ ID NOs: 77-80. In some instances, the humanized antibody or binding fragment thereof comprises a VH region comprising at least 80%, 85%, 90%, 95%, or 100% sequence identity to SEQ ID NOs: 81-84 and a VL region comprising at least 80%, 85%, 90%, 95%, or 100% sequence identity to SEQ ID NOs: 85-88. In some instances, the humanized antibody or binding fragment thereof comprises a VH region comprising at least 80%, 85%, 90%, 95%, or 100% sequence identity to SEQ ID NOs: 89-92 and a VL region comprising at least 80%, 85%, 90%, 95%, or 100% sequence identity to SEQ ID NOs: 93-97.

In some embodiments, the VH region and the VL region of a humanized anti-CLDN18.2 antibody is illustrated in Table 5. The underlined regions denote the respective CDR1, CDR2, or CDR3 sequence.

| | | SEQ |
|---|---|---|
| | | ID |
| NAME | SEQUENCE | NO: |
| 282A12_ | QVQLQESGPGLVKPSETLSLTCTVSGFSLTSYNVYWI | 65 |
| VHg0 | RQPPGKGLEWIGVIWNTGATRYNSALKSRVTISVDTS | |
| | KNQFSLKLSSVTAADTAVYYCARDSAMPAIPFAYWGQ | |
| | GTLVTVSS | |
| 282A12_ | QVQLQESGPGLVKPSETLSLTCTVSGFSLTSYNVYWI | 66 |
| VHg1 | RQPPGKGLEWIGVIWNTGATRYNSALKSRVTISKDTS | |
| | KNQVSLKLSSVTAADTAVYYCARDSAMPAIPFAYWGQ | |
| | GTLVTVSS | |
| 282A12_ | QVQLQESGPGLVKPSETLSLTCTVSGFSLTSYNVYWI | 67 |
| VHg2 | RQPPGKGLEWIGVIWNTGATRYNSALKSRVTISKDTS | |
| | KSQVSLKLSSVTAADTAVYYCARDSAMPAIPFAYWGQ | |
| | GTLVTVSS | |
| 282A12_ | QVQLQESGPGLVKPSETLSLTCTVAGFSLTSYNVYWI | 68 |
| VHg3 | RQPPGKGLEWIGVIWNTGATRYNSALKSRVTISKDTS | |
| | KSQVSLKLSSVTAADTAVYYCARDSAMPAIPFAYWGQ | |
| | GTLVTVSS | |
| 282A12_ | DIVMTQSPDSLAVSLGERATINCKSSQSLFGSVRQKN | 69 |
| VLg1 | YLAWYQQKPGQPPKLLIYLASTRESGVPDRFSGSGSG | |
| | TDFTLTISSLQAEDVAVYYCQQYYDIPWTFGGGTKVE | |
| | IK | |
| 282A12_ | DIVMTQSPDSLAVSLGERATINCKSSQSLFGSVRQKN | 70 |
| VLg1 | YLAWYQQKPGQSPKLLIYLASTRESGVPDRFSGSGSG | |
| | TDFTLTISSLQAEDVAKYYCQQYYDIPWTFGGGTKVE | |
| | IK | |
| 282A12_ | DIQMTQSPSSLSASVGDRVTITCKSSQSLFGSVRQKN | 71 |
| VLg2 | YLAWYQQKPGKAPKLLIYLASTRESGVPSRFSGSGSG | |
| | TDFTLTISSLQPEDFATYYCQQYYDIPWTFGGGTKVE | |
| | IK | |
| 282A12_ | DIQMTQSPSSLSASVGDRVTITCKSSQSLFGSVRQKN | 72 |
| VLg3 | YLAWYQQKPGKSPKLLIYLASTRESGVPDRFSGSGSG | |
| | TDFTLTISSLQPEDFAKYYCQQYYDIPWTFGGGTKVE | |
| | IK | |
| 282A12_ | DIVMTQSPSSLSASVGDRVTINCKSSQSLFGSVRQKN | 73 |
| VLg4 | YLAWYQQKPGKSPKLLIYLASTRESGVPDRFSGSGSG | |
| | TDFTLTISSLQPEDFAKYYCQQYYDIPWTFGGGTKVE | |
| | IK | |
| 413H9F8_ | EVQLVESGGGLVQPGGSLRLSCAASGFTFSSFGMHWV | 74 |
| VHg0 | RQAPGKGLEWVSYISSGSSPIYYVDKLKGRFTISRDN | |
| | AKNSLYLQMNSLRAEDTAVYYCARAGYAVRNALDYWG | |
| | QGTLVTVSS | |
| 413H9F8_ | DVQLVESGGGLVQPGGSLRLSCAASGFTFSSFGMHWV | 75 |
| VHg1 | RQAPGKGLEWVAYISSGSSPIYYVDKLKGRFTISRDN | |
| | AKNSLYLQMNSLRAEDTAVYYCARAGYAVRNALDYWG | |
| | QGTLVTVSS | |
| 413H9F8_ | DVQLVESGGGLVQPGGSLRLSCAASGFTFSSFGMHWV | 76 |
| VHg2 | RQAPGKGLEWVAYISSGSSPIYYVDKLKGRFTVSRDN | |
| | AKNSLYLQMNSLRAEDTAVYYCARAGYAVRNALDYWG | |
| | QGTLVTVSS | |
| 413H9F8_ | EIVMTQSPPTLSLSPGERVTLSCKSSQSLLNVGNQKN | 77 |
| VLg0 | YLAWYQQKPGQAPRLLIYGASTRESGIPARFSGSGSG | |
| | TDFTLTISSLQPEDFAVYYCQNDLFYPLTFGGGTKVE | |
| | IK | |
| 413H9F8_ | DIVMTQSPPTLSLSPGERVTLSCKSSQSLLNVGNQKN | 78 |
| VLg1 | YLAWYQQKPGQAPRLLIYGASTRESGIPDRFSGSGSG | |
| | TDFTLTISSLQPEDFAVYYCQNDLFYPLTFGGGTKVE | |
| | IK | |
| 413H9F8_ | DIVMTQSPPTLSLSPGERVTLSCKSSQSLLNVGNQKN | 79 |
| VLg2 | YLAWYQQKPGQAPKLLIYGASTRESGIPDRFSGSGSG | |
| | TDFTLTISSLQPEDFAVYYCQNDLFYPLTFGGGTKVE | |
| | IK | |
| 413H9F8_ | DIVMTQSPPTLSLSPGERVTLSCKSSQSLLNVGNQKN | 80 |
| VLg3 | YLAWYQQKPGQAPKLLIYGASTRESGIPDRFSGSGSG | |
| | TDFTLTISSLQPEDFAVYFCQNDLFYPLTFGGGTKVE | |
| | IK | |
| 413H9F8_ | EVQLVESGGGLVQPGGSLRLSCAASGFTFSSFGMHWV | 81 |
| HC-V1 | RQAPGKGLEWVSYISSGSSPIYYVDKLKGRFTISRDN | |
| | AKNSLYLQMNSLRAEDTAVYYCARAGYAVRNALDYWG | |
| | QGTLVTVSS | |
| 413H9F8_ | EVQLVESGGGLVQPGGSLRLSCAASGFTFSSFGMHWV | 82 |
| HC-V2 | RQAPGKGLEWVAYISSGSSPIYYVDKLKGRFTISRDN | |
| | AKNSLYLQMNSLRAEDTAVYYCARAGYAVRNALDYWG | |
| | QGTLVTVSS | |
| 413H9F8_ | EVQLVESGGGLVQPGGSLRLSCAASGFTFSSFGMHWV | 83 |
| HC-V3 | RQAPGKGLEWVAYISSGSSPIYYVDKLKGRFTVSRDN | |
| | AKNSLYLQMNSLRAEDTAVYYCARAGYAVRNALDYWG | |
| | QGTLVTVSS | |
| 413H9F8_ | EVQLVESGGGLVQPGGSLRLSCAASGFTFSSFGMHWV | 84 |
| HC-V4 | RQAPGKGLEWVAYISSGSSPIYYVDKLKGRFTVSRDN | |
| | AKNSLYLQMTSLRAEDTAVYYCARAGYAVRNALDYWG | |
| | QGTLVTVSS | |
| 413H9F8_ | DIVMTQSPDSLAVSLGERATINCKSSQSLLNVGNQKN | 85 |
| LC-V1 | YLAWYQQKPGQPPKLLIYGASTRESGVPDRFSGSGSG | |
| | TDFTLTISSLQAEDVAVYYCQNDLFYPLTFGGGTKVE | |
| | IK | |
| 413H9F8_ | DIVMTQSPDSLAVSLGERATINCKSSQSLLNVGNQKN | 86 |
| LC-V2 | YLAWYQQKPGQPPKLLIYGASTRESGVPDRFSGSGSG | |
| | TDFTLTISSLQAEDVAVYFCQNDLFYPLTFGGGTKVE | |
| | IK | |
| 413H9F8_ | DIVMTQSPDSLAVSLGERATINCKSSQSLLNVGNQKN | 87 |
| LC-V3 | YLAWYQQKPGQPPKLLIYGASTRESGVPDRFIGSGSG | |
| | TDFTLTISSLQAEDVAVYFCQNDLFYPLTFGGGTKVE | |
| | IK | |
| 413H9F8_ | DIVMTQSPDSLAVSLGERATISCKSSQSLLNVGNQKN | 88 |
| LC-V4 | YLAWYQQKPGQPPKLLIYGASTRESGVPDRFIGSGSG | |
| | TDFTLTISSLQAEDVAVYFCQNDLFYPLTFGAGTKVE | |
| | IK | |
| 364D1A7_ | EVQLVESGGGLVQPGGSLRLSCAASGFTFSSFGMHWV | 89 |
| HC_V1 | RQAPGKGLEWVSYISSGSGSIYYADTVKGRFTISRDN | |
| | AKNSLYLQMNSLRAEDTAVYYCATSYYYGNALEYWGQ | |
| | GTTVTVSS | |
| 364D1A7_ | EVQLVESGGGLVQPGGSLRLSCAASGFTFSSFGMHWV | 90 |
| HC_V2 | RQAPGKGLEWVAYISSGSGSIYYADTVKGRFTISRDN | |
| | PKNSLYLQMNSLRAEDTAVYYCATSYYYGNALEYWGQ | |
| | GTTVTVSS | |
| 364D1A7_ | EVQLVESGGGLVQPGGSLRLSCAASGFTFSSFGMHWV | 91 |
| HC_V3 | RQAPGKGLEWVAYISSGSGSIYYADTVKGRFTLSRDN | |
| | PKNSLYLQMNSLRAEDTAVYYCATSYYYGNALEYWGQ | |
| | GTTVTVSS | |
| 364D1A7_ | DVQLVESGGGLVQPGGSLRLSCAASGFTFSSFGMHWV | 92 |
| HC_V4 | RQAPGKGLEWVAYISSGSGSIYYADTVKGRFTLSRDN | |
| | PKNTLYLQMNSLRAEDTAVYYCATSYYYGNALEYWGQ | |
| | GTTVTVSS | |
| 364D1A7_ | DIVMTQSPDSLAVSLGERATINCKSSQSLFNVGNQKN | 93 |
| LC_V1 | YLTWYQQKPGQPPKLLIYWASTRKSGVPDRFSGSGSG | |
| | TDFTLTISSLQAEDVAVYYCQNVYSYPLTFGGGTKVE | |
| | IK | |
| 364D1A7_ | DIVLTQSPDSLAVSLGERATINCKSSQSLFNVGNQKN | 94 |
| LC_V2 | YLTWYQQKPGQTPKLLIYWASTRKSGVPDRFSGSGSG | |
| | TDFTLTISSLQAEDVAVYYCQNVYSYPLTFGGGTKVE | |
| | IK | |
| 364D1A7_ | DVVLTQSPDSLAVSLGERATINCKSSQSLFNVGNQKN | 95 |
| LC_V3 | YLTWYQQKPGQTPKLLIYWASTRKSGVPDRFSGSGSG | |
| | TDFTLTISSLQAEDVAVYYCQNVYSYPLTFGGGTKVE | |
| | IK | |
| 364D1A7_ | DVVLTQSPDSLAVSLGERATISCKSSQSLFNVGNQKN | 96 |
| LC_V4 | YLTWYQQKPGQTPTLLIYWASTRKSGVPDRFSGSGSG | |
| | TDFTLTISSLQAEDVAVYYCQNVYSYPLTFGGGTKVE | |
| | IK | |
| 364D1A7_ | DVVLTQSPDSLAVSLGERATISCKSSQSLFNVGNQKN | 97 |
| LC_V5 | YLTWYQQKPGQTPTLLIYWASTRKSGVPDRFTGSGSG | |
| | TDFTLTISSLQAEDVAVYYCQNVYSYPLTFGAGTKVE | |
| | IK | |

In some embodiments, an anti-CLDN18.2 antibody described herein comprises a VH region and a VL region as illustrated in Table 6.

| | 282A12_VH g0 | 282A12_VH g1 | 282A12_VH g2 | 282A12_VH g3 |
|---|---|---|---|---|
| | (SEQ ID NO: 65) | (SEQ ID NO: 66) | (SEQ ID NO: 67) | (SEQ ID NO: 68) |
| 282A12_VL g0 | hz282-1 | hz282-2 | hz282-3 | hz282-4 |
| (SEQ ID NO: 69) | (SEQ ID NO: 65 + | (SEQ ID NO: 66 + | (SEQ ID NO: 67 + | (SEQ ID NO: 68 + |
| | SEQ ID NO: 69) | SEQ ID NO: 69) | SEQ ID NO: 69) | SEQ ID NO: 69) |
| 282A12_VL g1 | hz282-5 | hz282-6 | hz282-7 | hz282-8 |
| (SEQ ID NO: 70) | (SEQ ID NO: 65 + | (SEQ ID NO: 66 + | (SEQ ID NO: 67 + | (SEQ ID NO: 68 + |
| | SEQ ID NO: 70) | SEQ ID NO: 70) | SEQ ID NO: 70) | SEQ ID NO: 70) |
| 282A12_VL g2 | hz282-9 | hz282-10 | hz282-11 | hz282-12 |
| (SEQ ID NO: 71) | (SEQ ID NO: 65 + | (SEQ ID NO: 66 + | (SEQ ID NO: 67 + | (SEQ ID NO: 68 + |
| | SEQ ID NO: 71) | SEQ ID NO: 71) | SEQ ID NO: 71) | SEQ ID NO: 71) |
| 282A12_VL g3 | hz282-13 | hz282-14 | hz282-15 | hz282-16 |
| (SEQ ID NO: 72) | (SEQ ID NO: 65 + | (SEQ ID NO: 66 + | (SEQ ID NO: 67 + | (SEQ ID NO: 68 + |
| | SEQ ID NO: 72) | SEQ ID NO: 72) | SEQ ID NO: 72) | SEQ ID NO: 72) |
| 282A12_VL g4 | hz282-17 | hz282-18 | hz282-19 | hz282-20 |
| (SEQ ID NO: 73) | (SEQ ID NO: 65 + | (SEQ ID NO: 66 + | (SEQ ID NO: 67 + | (SEQ ID NO: 68 + |
| | SEQ ID NO: 73) | SEQ ID NO: 73) | SEQ ID NO: 73) | SEQ ID NO: 73) |

In some embodiments, an anti-CLDN18.2 antibody described herein comprises a VH region and a VL region as illustrated in Table 7.

| | 413H9F8_VH g0 | 413H9F8_VH g1 | 413H9F8_VH g2 |
|---|---|---|---|
| | (SEQ ID NO: 74) | (SEQ ID NO: 75) | (SEQ ID NO: 76) |
| 413H9F8_VL g0 | 413H9F8-cp1 | 413H9F8-cp2 | 413H9F8-cp3 |
| (SEQ ID NO: 77) | (SEQ ID NO: 74 + | SEQ ID NO: 75 + | SEQ ID NO: 76 + |
| | SEQ ID NO: 77) | SEQ ID NO: 77) | SEQ ID NO: 77) |
| 413H9F8_VL g1 | 413H9F8-cp4 | 413H9F8-cp5 | 413H9F8-cp6 |
| (SEQ ID NO: 78) | (SEQ ID NO: 74 + | (SEQ ID NO: 75 + | (SEQ ID NO: 76 + |
| | SEQ ID NO: 78) | SEQ ID NO: 78) | SEQ ID NO: 78) |
| 413H9F8_VL g2 | 413H9F8-cp7 | 413H9F8-cp8 | 413H9F8-cp9 |
| (SEQ ID NO: 79) | (SEQ ID NO: 74 + | (SEQ ID NO: 75 + | (SEQ ID NO: 76 + |
| | SEQ ID NO: 79) | SEQ ID NO: 79) | SEQ ID NO: 79) |
| 413H9F8_VL g3 | 413H9F8-cp10 | 413H9F8-cp11 | 413H9F8-cp12 |
| (SEQ ID NO: 80) | (SEQ ID NO: 74 + | (SEQ ID NO: 75 + | (SEQ ID NO: 76 + |
| | SEQ ID NO: 80) | SEQ ID NO: 80) | SEQ ID NO: 80) |

In some embodiments, an anti-CLDN18.2 antibody described herein comprises a VH region and a VL region as illustrated in Table 8.

| | 413H9F8_LC-V1 | 413H9F8_LC-V2 | 413H9F8_LC-V3 | 413H9F8_LC-V4 |
|---|---|---|---|---|
| | (SEQ ID NO: 85) | (SEQ ID NO: 86) | (SEQ ID NO: 87) | (SEQ ID NO: 88) |
| 413H9F8_HC-V1 | 413H9F8-H1L1 | 413H9F8-H1L2 | 413H9F8-H1L3 | 413H9F8-H1L4 |
| (SEQ ID NO: 81) | (SEQ ID NO: 85 + | (SEQ ID NO: 86 + | (SEQ ID NO: 87 + | (SEQ ID NO: 88 + |
| | SEQ ID NO: 81) | SEQ ID NO: 81) | SEQ ID NO: 81) | SEQ ID NO: 81) |
| 413H9F8_HC-V2 | 413H9F8-H2L1 | 413H9F8-H2L2 | 413H9F8-H2L3 | 413H9F8-H2L4 |
| (SEQ ID NO: 82) | (SEQ ID NO: 85 + | (SEQ ID NO: 86 + | (SEQ ID NO: 87 + | (SEQ ID NO: 88 + |
| | SEQ ID NO: 82) | SEQ ID NO: 82) | SEQ ID NO: 82) | SEQ ID NO: 82) |
| 413H9F8_HC-V3 | 413H9F8-H3L1 | 413H9F8-H3L1 | 413H9F8-H3L1 | 413H9F8-H3L1 |
| (SEQ ID NO: 83) | (SEQ ID NO: 85 + | (SEQ ID NO: 86 + | (SEQ ID NO: 87 + | (SEQ ID NO: 88 + |
| | SEQ ID NO: 83) | SEQ ID NO: 83) | SEQ ID NO: 83) | SEQ ID NO: 83) |
| 413H9F8_HC-V4 | 413H9F8-H4L1 | 413H9F8-H4L2 | 413H9F8-H4L3 | 413H9F8-H4L4 |
| (SEQ ID NO: 84) | (SEQ ID NO: 85 + | (SEQ ID NO: 86 + | (SEQ ID NO: 87 + | (SEQ ID NO: 88 + |
| | SEQ ID NO: 84) | SEQ ID NO: 84) | SEQ ID NO: 84) | SEQ ID NO: 84) |

In some embodiments, an anti-CLDN18.2 antibody described herein comprises a VH region and a VL region as illustrated in Table 9.

| | 364D1A7_LC_V1 | 364D1A7_LC_V2 | 364D1A7_LC_V3 | 364D1A7_LC_V4 | 364D1A7_LC_V5 |
|---|---|---|---|---|---|
| | (SEQ ID NO: 93) | (SEQ ID NO: 94) | (SEQ ID NO: 95) | (SEQ ID NO: 96) | (SEQ ID NO: 97) |
| 364D1A7_HC_V1 | 364D1A7_H1L1 | 364D1A7_H1L2 | 364D1A7_H1L3 | 364D1A7_H1L4 | 364D1A7_H1L5 |
| (SEQ ID NO: 89) | (SEQ ID NO: 93 + | (SEQ ID NO: 94 + | (SEQ ID NO: 95 + | (SEQ ID NO: 96 + | (SEQ ID NO: 97 + |
| | SEQ ID NO: 89) | SEQ ID NO: 89) | SEQ ID NO: 89) | SEQ ID NO: 89) | SEQ ID NO: 89) |
| 364D1A7_HC_V2 | 364D1A7_H2L1 | 364D1A7_H2L2 | 364D1A7_H2L3 | 364D1A7_H2L4 | 364D1A7_H2L5 |
| (SEQ ID NO: 90) | (SEQ ID NO: 93 + | (SEQ ID NO: 94 + | (SEQ ID NO: 95 + | (SEQ ID NO: 96 + | (SEQ ID NO: 97 + |
| | SEQ ID NO: 90) | SEQ ID NO: 90) | SEQ ID NO: 90) | SEQ ID NO: 90) | SEQ ID NO: 90) |
| 364D1A7_HC_V3 | 364D1A7_H3L1 | 364D1A7_H3L2 | 364D1A7_H3L3 | 364D1A7_H3L4 | 364D1A7_H3L5 |
| (SEQ ID NO: 91) | (SEQ ID NO: 93 + | (SEQ ID NO: 94 + | (SEQ ID NO: 95 + | (SEQ ID NO: 96 + | (SEQ ID NO: 97 + |
| | SEQ ID NO: 91) | SEQ ID NO: 91) | SEQ ID NO: 91) | SEQ ID NO: 91) | SEQ ID NO: 91) |
| 364D1A7_HC_V4 | 364D1A7_H4L1 | 364D1A7_H4L2 | 364D1A7_H4L3 | 364D1A7_H4L4 | 364D1A7_H4L5 |
| (SEQ ID NO: 92) | (SEQ ID NO: 93 + | (SEQ ID NO: 94 + | (SEQ ID NO: 95 + | (SEQ ID NO: 96 + | (SEQ ID NO: 97 + |
| | SEQ ID NO: 92) | SEQ ID NO: 92) | SEQ ID NO: 92) | SEQ ID NO: 92) | SEQ ID NO: 92) |

In some embodiments, an anti-CLDN18.2 antibody described herein comprises a framework region selected from IgM, IgG (e.g., IgG1, IgG2, IgG3, or IgG4), IgA, or IgE. In some cases, the anti-CLDN18.2 antibody comprises an IgM framework. In some cases, the anti-CLDN18.2 antibody comprises an IgG (e.g., IgG1, IgG2, IgG3, or IgG4) framework. In some cases, the anti-CLDN18.2 antibody comprises an IgG1 framework. In some cases, the anti-CLDN18.2 antibody comprises an IgG2 framework.

In some embodiments, the anti-CLDN18.2 antibody comprises one or more mutations in the framework region, e.g., in the CH1 domain, CH2 domain, CH3 domain, hinge region, or a combination thereof. In some cases, the one or more mutations modulate Fc receptor interactions, e.g., to increase Fc effector functions such as ADCC and/or complement-dependent cytotoxicity (CDC). In some cases, the one or more mutations stabilize the antibody and/or increase the half-life of the antibody. In additional cases, the one or more mutations modulate glycosylation.

In some embodiments, the Fc region comprises one or more mutations that modulate Fc receptor interactions, e.g., to enhance effector functions such as ADCC and/or CDC. In such instances, exemplary residues when mutated modulate effector functions include S239, F243, R292, Y300, V305, P396, K326, A330, I332, or E333, in which the residue position correspond to IgG1 and the residue numbering is in accordance to Kabat numbering (EU index of Kabat et al 1991 Sequences of Proteins of Immunological Interest). In some instances, the one or more mutations comprise S239D, F243L, R292P, Y300L, V305I, P396L, K326W, A330L, I332E, E333A, E333S, or a combination thereof. In some cases, the one or more mutations comprise S239D, I332E, or a combination thereof. In some cases, the one or more mutations comprises F243L, R292P, Y300L, V305I, P396L, I332E, or a combination thereof. In some cases, the one or more mutations comprise S239D, A330L, I332E, or a combination thereof. In some cases, the one or more mutations comprise K326W, E333S, or a combination thereof. In some cases, the mutation comprises E333A.

In some cases, the anti-CLDN18.2 antibody shares a binding epitope with the reference antibody 175D10.

In some cases, the anti-CLDN18.2 antibody has a cross-binding activity to mouse and cynomolgus CLDN18.2 protein.

### Antibody Production

In some embodiments, anti-CLDN18.2 antibodies are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-CLDN18.2 antibodies can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, incorporated fully herein by reference. Such antibodies can be referred to as human xenogenic antibodies.

Alternatively, anti-CLDN18.2 antibodies can be produced from phage libraries containing human variable regions. See U.S. Pat. No. 6,174,708, incorporated fully herein by reference.

In some aspects of any of the embodiments disclosed herein, an anti-CLDN18.2 antibody is produced by a hybridoma.

For monoclonal anti-CLDN18.2 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-CLDN18.2 antibody may be produced by genetic engineering.

Anti-CLDN18.2 antibodies disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-CLDN18.2 antibodies include, but are not limited to, humanized, chimeric, or xenogenic human anti-CLDN18.2 antibodies.

Chimeric anti-CLDN18.2 antibodies can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains, and incorporate only the complementarity-determining regions of the animal-derived antibody. This can be accomplished by carefully examining the sequence of the hyper-variable loops of the variable regions of a monoclonal antigen binding unit or monoclonal antibody, and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions, and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983)J Mol. Biol. 168:595) are available to predict the ideal sequence for the V region. The invention thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755.

Humanized antibodies can be prepared by a process of analysis of the parental sequences and various conceptual humanized products using three dimensional models of the parental and humanized sequences. Three dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

As noted above, an anti-CLDN18.2 antibody can be either “monovalent” or “multivalent.” Whereas the former has one binding site per antigen-binding unit, the latter contains multiple binding sites capable of binding to more than one antigen of the same or different kind. Depending on the number of binding sites, antigen binding units may be bivalent (having two antigen-binding sites), trivalent (having three antigen-binding sites), tetravalent (having four antigen-binding sites), and so on.

Multivalent anti-CLDN18.2 antibodies can be further classified on the basis of their binding specificities. A “monospecific” anti-CLDN18.2 antibody is a molecule capable of binding to one or more antigens of the same kind. A “multispecific” anti-CLDN18.2 antibody is a molecule having binding specificities for at least two different antigens. While such molecules normally will only bind two distinct antigens (i.e. bispecific anti-CLDN18.2 antibodies), antibodies with additional specificities such as trispecific antibodies are encompassed by this expression when used herein. This disclosure further provides multispecific anti-CLDN18.2 antibodies. Multispecific anti-CLDN18.2 antibodies are multivalent molecules capable of binding to at least two distinct antigens, e.g., bispecific and trispecific molecules exhibiting binding specificities to two and three distinct antigens, respectively.

### Polynucleotides and Vectors

In some embodiments, the present disclosure provides isolated nucleic acids encoding any of the anti-CLDN18.2 antibodies disclosed herein. In another embodiment, the present disclosure provides vectors comprising a nucleic acid sequence encoding any anti-CLDN18.2 antibody disclosed herein. In some embodiments, this invention provides isolated nucleic acids that encode a light-chain CDR and a heavy-chain CDR of an anti-CLDN18.2 antibody disclosed herein.

The subject anti-CLDN18.2 antibodies can be prepared by recombinant DNA technology, synthetic chemistry techniques, or a combination thereof. For instance, sequences encoding the desired components of the anti-CLDN18.2 antibodies, including light chain CDRs and heavy chain CDRs are typically assembled cloned into an expression vector using standard molecular techniques know in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-CLDN18.2 antibody of interest.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection (atcc.org), which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-CLDN18.2 antibodies can also be modified, for example, by substituting the coding sequence for human heavy and light chain constant regions in place of the homologous non-human sequences. In that manner, chimeric antibodies are prepared that retain the binding specificity of the original anti-CLDN18.2 antibody.

It is also understood that the polynucleotides embodied in the disclosure include those coding for functional equivalents and fragments thereof of the exemplified polypeptides. Functionally equivalent polypeptides include those that enhance, decrease or not significantly affect properties of the polypeptides encoded thereby. Functional equivalents may be polypeptides having conservative amino acid substitutions, analogs including fusions, and mutants.

Due to the degeneracy of the genetic code, there can be considerable variation in nucleotides of an antigen binding unit coding sequence, as well as sequences suitable for construction of the polynucleotide and vectors of the present invention. Sequence variants may have modified DNA or amino acid sequences, one or more substitutions, deletions, or additions, the net effect of which is to retain the desired antigen-binding activity. For instance, various substitutions can be made in the coding region that either do not alter the amino acids encoded or result in conservative changes. These substitutions are encompassed by the present invention. Conservative amino acid substitutions include substitutions within the following groups: glycine, alanine; valine, isoleucine, leucine; aspartic acid, glutamic acid; asparagine, glutamine; serine, threonine; lysine, arginine; and phenylalanine, tyrosine. While conservative substitutions do effectively change one or more amino acid residues contained in the polypeptide to be produced, the substitutions are not expected to interfere with the antigen-binding activity of the resulting antigen binding units to be produced. Nucleotide substitutions that do not alter the amino acid residues encoded are useful for optimizing gene expression in different systems. Suitable substitutions are known to those of skill in the art and are made, for instance, to reflect preferred codon usage in the expression systems.

Where desired, the recombinant polynucleotides may comprise heterologous sequences that facilitate detection of the expression and purification of the gene product. Examples of such sequences are known in the art and include those encoding reporter proteins such as β-galactosidase, β-lactamase, chloramphenicol acetyltransferase (CAT), luciferase, green fluorescent protein (GFP) and their derivatives. Other heterologous sequences that facilitate purification may code for epitopes such as Myc, HA (derived from influenza virus hemagglutinin), His-6, FLAG, or the Fc portion of immunoglobulin, glutathione S-transferase (GST), and maltose-binding protein (MBP).

Polynucleotides disclosed herein can be conjugated to a variety of chemically functional moieties described above. Commonly employed moieties include labels capable of producing a detectable signal, signal peptides, agents that enhance immunologic reactivity, agents that facilitate coupling to a solid support, vaccine carriers, bioresponse modifiers, paramagnetic labels and drugs. The moieties can be covalently linked polynucleotide recombinantly or by other means known in the art.

Polynucleotides disclosed herein can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

Polynucleotides disclosed herein can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

Polynucleotides comprising a desired sequence can be inserted into a suitable vector which in turn can be introduced into a suitable host cell for replication and amplification. Accordingly, a variety of vectors comprising one or more of the polynucleotides described above are contemplated herein. Also provided are selectable libraries of expression vectors comprising at least one vector encoding an anti-CLDN18.2 antibody disclosed herein.

Vectors generally comprise transcriptional or translational control sequences required for expressing the antigen binding units. Suitable transcription or translational control sequences include but are not limited to replication origin, promoter, enhancer, repressor binding regions, transcription initiation sites, ribosome binding sites, translation initiation sites, and termination sites for transcription and translation.

The choice of promoters will largely depend on the host cells in which the vector is introduced. It is also possible, to utilize promoters normally associated with a desired light or heavy chain gene, provided that such control sequences are compatible with the host cell system. Cell-specific or tissue-specific promoters may also be used. A vast diversity of tissue specific promoters have been described and employed by artisans in the field. Exemplary promoters operative in selective animal cells include hepatocyte-specific promoters and cardiac muscle specific promoters. Depending on the choice of the recipient cell types, those skilled in the art will know of other suitable cell-specific or tissue-specific promoters applicable for the construction of the expression vectors of the present invention.

Using known molecular cloning or gene engineering techniques, appropriate transcriptional control sequences, enhancers, terminators, or any other genetic element known in the art can integrated in operative relationship, optionally additionally with intact selectable fusion genes to be expressed in accordance with the present invention. In addition to the above-described elements, the vectors may contain a selectable marker (for example, a gene encoding a protein necessary for the survival or growth of a host cell transformed with the vector), although such a marker gene can be carried on another polynucleotide sequence co-introduced into the host cell.

The polynucleotides and vectors described herein have several specific uses. They are useful, for example, in expression systems for the production of antigen binding units. Such polynucleotides are useful as primers to effect amplification of desired polynucleotides. Furthermore, polynucleotides are also useful in pharmaceutical compositions including vaccines, diagnostics, and drugs.

The host cells can be used, inter alia, as repositories of the subject polynucleotides, vectors, or as vehicles for producing and screening desired anti-CLDN18.2 antibodies based on their antigen binding specificities.

Accordingly, the disclosure provides a method of identifying an anti-CLDN18.2 antibody that is immunoreactive with a desired antigen. Such a method can involve the following steps: (a) preparing a genetically diverse library of anti-CLDN18.2 antibodies, wherein the library comprises at least one subject anti-CLDN18.2 antibody; (b) contacting the library of anti-CLDN18.2 antibodies with the desired antigen; (c) detecting a specific binding between anti-CLDN18.2 antibodies and the antigen, thereby identifying the anti-CLDN18.2 antibody that is immunoreactive with the desired antigen. 0102 The ability of an anti-CLDN18.2 antibody to specifically bind to a desired antigen can be tested by a variety of procedures well established in the art. See Harlow and Lane (1988) Antibodies: A Laboratory Manual, Cold Spring Harbor Laboratory, New York; Gherardi et al. (1990) J. Immunol. Meth. 126:61-68. Typically, anti-CLDN18.2 antibodies exhibiting desired binding specificities can be detected directly by immunoassays, for example, by reacting labeled anti-CLDN18.2 antibodies with the antigens that are immobilized on a solid support or substrate. In general, the substrate to which the antigen is adhered is fabricated with material exhibiting a low level of non-specific binding during immunoassay. An example solid support is made from one or more of the following types of materials: plastic polymers, glass, cellulose, nitrocellulose, semi-conducting material, and metal. In some examples, the substrate is petri dish, chromatography beads, magnetic beads, and the like.

For such solid-phase assays, the unreacted anti-CLDN18.2 antibodies are removed by washing. In a liquid-phase assay, however, the unreacted anti-CLDN18.2 antibodies are removed by some other separation technique, such as filtration or chromatography. After binding the antigen to the labeled anti-CLDN18.2 antibodies, the amount of bound label is determined. A variation of this technique is a competitive assay, in which the antigen is bound to saturation with an original binding molecule. When a population of the subject anti-CLDN18.2 antibody is introduced to the complex, only those that exhibit higher binding affinity will be able to compete, and thus remain bound to the antigen.

Alternatively, specific binding to a given antigen can be assessed by cell sorting, which involves presenting the desired antigen on the cells to be sorted, then labeling the target cells with anti-CLDN18.2 antibodies that are coupled to detectable agents, followed by separating the labeled cells from the unlabeled ones in a cell sorter. A sophisticated cell separation method is fluorescence-activated cell sorting (FACS). Cells traveling in single file in a fine stream are passed through a laser beam, and the fluorescence of each cell bound by the fluorescently labeled anti-CLDN18.2 antibody is then measured.

Subsequent analysis of the eluted anti-CLDN18.2 antibodies may involve protein sequencing for delineating the amino acid sequences of the light chains and heavy chains. Based on the deduced amino acid sequences, the cDNA encoding the anti-CLDN18.2 antibodies can then be obtained by recombinant cloning methods including PCR, library screening, homology searches in existing nucleic acid databases, or any combination thereof. Commonly employed databases include but are not limited to GenBank, EMBL, DDBJ, PDB, SWISS-PROT, EST, STS, GSS, and HTGS.

When a library of anti-CLDN18.2 antibodies is displayed on phage or bacterial particles, selection is preferably performed using affinity chromatography. The method typically proceeds with binding a library of phage anti-CLDN18.2 antibodies to an antigen coated plates, column matrices, cells or to biotinylated antigen in solution followed by capture. The phages or bacteria bound to the solid phase are washed and then eluted by soluble hapten, acid or alkali. Alternatively, increasing concentrations of antigen can be used to dissociate the anti-CLDN18.2 antibodies from the affinity matrix. For certain anti-CLDN18.2 antibodies with extremely high affinity or avidity to the antigen, efficient elution may require high pH or mild reducing solution as described in WO 92/01047.

The efficiency of selection is likely to depend on a combination of several factors, including the kinetics of dissociation during washing, and whether multiple anti-CLDN18.2 antibodies on a single phage or bacterium can simultaneously bind to antigens on a solid support. For example, antibodies with fast dissociation kinetics (and weak binding affinities) can be retained by use of short washes, multivalent display and a high coating density of antigen at the solid support. Conversely, the selection of anti-CLDN18.2 antibodies with slow dissociation kinetics (and good binding affinities) can be favored by use of long washes, monovalent phages, and a low coating density of antigen.

Where desired, the library of anti-CLDN18.2 antibodies can be pre-selected against an unrelated antigen to counter-select the undesired antibodies. The library may also be pre-selected against a related antigen in order to isolate, for example, anti-idiotypic antibodies.

### Host Cells

In some embodiments, the present disclosure provides host cells expressing any one of the anti-CLDN18.2 antibodies disclosed herein. A subject host cell typically comprises a nucleic acid encoding any one of the anti-CLDN18.2 antibodies disclosed herein.

The invention provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-CLDN18.2 antibodies can be determined using any nucleic acid or protein assay known in the art. For example, the presence of transcribed mRNA of light chain CDRs or heavy chain CDRs, or the anti-CLDN18.2 antibody can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-CLDN18.2 antibody.

Expression of the vector can also be determined by examining the expressed anti-CLDN18.2 antibody. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Payloads

In some embodiments, an anti-CLDN18.2 antibody described herein is further conjugated to a payload. In some instances, the payload is conjugated directly to the anti-CLDN18.2 antibody. In other instances, the payload is conjugated indirectly to the anti-CLDN18.2 antibody via a linker. In some cases, the payload comprises a small molecule, a protein or functional fragment thereof, a peptide, or a nucleic acid polymer.

In some cases, the number of payloads conjugated to the anti-CLDN18.2 antibody (e.g., the drug-to-antibody ratio or DAR) is about 1:1, one payload to one anti-CLDN18.2 antibody. In some cases, the ratio of the payloads to the anti-CLDN18.2 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-CLDN18.2 antibody is about 2:1. In some cases, the ratio of the payloads to the anti-CLDN18.2 antibody is about 3:1. In some cases, the ratio of the payloads to the anti-CLDN18.2 antibody is about 4:1. In some cases, the ratio of the payloads to the anti-CLDN18.2 antibody is about 6:1. In some cases, the ratio of the payloads to the anti-CLDN18.2 antibody is about 8:1. In some cases, the ratio of the payloads to the anti-CLDN18.2 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the tubulysin is a tubulysin analog or derivative such as described in U.S. Pat. Nos. 8,580,820 and 8,980,833 and in U.S. Publication Nos. 20130217638, 20130224228, and 201400363454.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycine A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SIG-136 (SG-2000), ZC-423 (SG2285), SJG-720, SJG-738, ZC-207 (SG2202), and DSB-120 (Table 2). In some embodiments, the PBD dimer is an unsymmetrical dimer. Examples of unsymmetrical PBD dimers include, but are not limited to, SIG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. Exemplary detectable moieties include fluorescent dyes; enzymes; substrates; chemiluminescent moieties; specific binding moieties such as streptavidin, avidin, or biotin; or radioisotopes.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “Si factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL—panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display improved properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modification include morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof.

### Conjugation Chemistry

KOI In some instances, the payload is conjugated to an anti-CLDN18.2 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-CLDN18.2 antibody described herein by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-CLDN18.2 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-CLDN18.2 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-CLDN18.2 antibody utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-CD38 antibody, an anti-ICAM1 antibody, or a multi-specific antibody (e.g., a bispecific anti-CD38/ICAM1 antibody) described herein by a method as described in PCT Publication No. WO2014/140317, which utilizes a sequence-specific transpeptidase.

In some instances, the payload is conjugated to an anti-CLDN18.2 antibody described herein by a method as described in U.S. Patent Publication Nos. 2015/0105539 and 2015/0105540.

### Linker

In some instances, a linker described above comprises a natural or synthetic polymer, consisting of long chains of branched or unbranched monomers, and/or cross-linked network of monomers in two or three dimensions. In some instances, the linker includes a polysaccharide, lignin, rubber, or polyalkylen oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide units. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) staring material in a step-wise fashion. In some cases, a dPEG has a specific molecular weight, rather than an average molecular weight. In some cases, a dPEG described herein is a dPEG from Quanta Biodesign, LMD.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some cases, the linker is a dPEG from Quanta Biodesign, LMD.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such as 1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Methods of Use

In certain embodiments, disclosed herein is a method of treating a subject having a cancer that is characterized with an overexpression of CLDN18.2 protein. In some cases, the method comprises administering to the subject an anti-CLDN18.2 antibody described herein or a pharmaceutical composition comprising an anti-CLDN18.2 antibody to treat the cancer in the subject. In some cases, the cancer is a gastrointestinal cancer. Exemplary gastrointestinal cancers include cancers of the esophagus, gallbladder and biliary tract, liver, pancreas, stomach, small intestine, large intestine, colon, rectum, and/or anus.

In some instances, the gastrointestinal cancer is stomach (or gastric) cancer. In some cases, the stomach (or gastric) cancer comprises adenocarcinomas of the stomach, gastric lymphoma, gastrointestinal stromal tumor (GIST), carcinoid tumor, squamous cell carcinoma, small cell carcinoma, or leiomyosarcoma.

In some instances, the gastrointestinal cancer is pancreatic cancer. In some cases, the pancreatic cancer comprises an exocrine tumor such as adenocarcinoma of the pancreas, acinar cell carcinoma, intraductal papillary-mucinous neoplasma (IPMN), or mucinous cystadenocarcinoma; or a pancreatic neuroendocrine tumor (PNET) (also known as islet cell tumor) such as gastrinoma, glucaganoma, insulinoma, somatostatinoma, VIPoma, or nonfunctional islet cell tumor.

In some instances, the gastrointestinal cancer is esophageal cancer. In some cases, the esophageal cancer comprises adenocarcinoma of the esophagus, squamous cell carcinoma, or small cell carcinoma.

In some instances, the gastrointestinal cancer is cholangiocarcinoma.

In some instances, the cancer is lung cancer. In some cases, the lung cancer comprises a non-small cell lung cancer (NSCLC) such as lung adenocarcinoma, squamous cell carcinoma, or large cell carcinoma; or small cell lung cancer (SCLC).

In some instances, the cancer is ovarian cancer. In some cases, the ovarian cancer comprises an epithelial ovarian tumor, an ovarian germ cell tumor, an ovarian stromal tumor, or a primary peritoneal carcinoma.

In some embodiments, the method further comprises administering to the subject an additional therapeutic agent. In some instances, the additional therapeutic agent comprises a chemotherapeutic agent, an immunotherapeutic agent, a targeted therapeutic agent, a hormone-based therapeutic agent, or a stem-cell based therapeutic agent.

In some instances, the additional therapeutic agent comprises a chemotherapeutic agent. Exemplary chemotherapeutic agents include, but are not limited to, alkylating agents such as cyclophosphamide, mechlorethamine, chlorambucil, melphalan, dacarbazine, or nitrosoureas; anthracyclines such as daunorubicin, doxorubicin, epirubicin, idarubicin, mitoxantrone, or valrubicin; cytoskeletal disruptors such as paclitaxel, docetaxel, abraxane, or taxotere; epothilones; histone deacetylase inhibitors such as vorinostat or romidepsin; topoisomerase I inhibitors such as irinotecan or topotecan; topoisomerase II inhibitors such as etoposide, teniposide, or tafluposide; kinase inhibitors such as bortezomib, erlotinib, gefitinib, imatinib, vemurafenib, or vismodegib; nucleotide analogs and precursor analogs such as azacitidine, azathioprine, capecitabine, cytarabine, doxifluridine, fluorouracil, gemcitabine, hydrozyurea, mercaptopurine, methotrexate, or tioguanine; platinum-based agents such as carboplatin, cisplatin, or oxaliplatin; retinoids such as tretinoin, alitretinoin, or bexarotene; or vinca alkaloids and derivatives such as vinblastine, vincristine, vindesine, or vinorelbine.

In some instances, the additional therapeutic agent comprises an immunotherapeutic agent. In some instances, the immunotherapy is an adoptive cell therapy. Exemplary adoptive cell therapies include AFP TCR, MAGE-A10 TCR, or NY-ESO-TCR from Adaptimmune; ACTR087/rituximab from Unum Therapeutics; anti-BCMA CAR-T cell therapy, anti-CD19 “armored” CAR-T cell therapy, JCAR014, JCAR018, JCAR020, JCAR023, JCAR024, or JTCR016 from Juno Therapeutics; JCAR017 from Celgene/Juno Therapeutics; anti-CD19 CAR-T cell therapy from Intrexon; anti-CD19 CAR-T cell therapy, axicabtagene ciloleucel, KITE-718, KITE-439, or NY-ESO-1 T-cell receptor therapy from Kite Pharma; anti-CEA CAR-T therapy from Sorrento Therapeutics; anti-PSMA CAR-T cell therapy from TNK Therapeutics/Sorrento Therapeutics; ATA520 from Atara Biotherapeutics; AU101 and AU105 from Aurora BioPharma; baltaleucel-T (CMD-003) from Cell Medica; bb2121 from bluebird bio; BPX-501, BPX-601, or BPX-701 from Bellicum Pharmaceuticals; BSK01 from Kiromic; IMCgp100 from Immunocore; JTX-2011 from Jounce Therapeutics; LN-144 or LN-145 from Lion Biotechnologies; MB-101 or MB-102 from Mustang Bio; NKR-2 from Celyad; PNK-007 from Celgene; tisagenlecleucel-T from Novartis Pharmaceuticals; or TT12 from Tessa Therapeutics.

In some instances, the immunotherapy is a dendritic cell-based therapy.

In some instances, the immunotherapy comprises a cytokine-based therapy, comprising e.g., an interleukin (IL) such as IL-2, IL-15, or IL-21, interferon (IFN)-α, or granulocyte macrophage colony-stimulating factor (GM-CSF).

In some instances, the immunotherapy comprises an immune checkpoint modulator. Exemplary immune checkpoint modulators include PD-1 modulators such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDI0680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 modulators such as ipilimumab (Yervoy), or AGEN 1884 from Agenus; PD-L1 modulators such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; LAG3 modulators such as BMS-986016 from Bristol-Myers Squibb, IMP701 from Novartis Pharmaceuticals, LAG525 from Novartis Pharmaceuticals, or REGN3767 from Regeneron Pharmaceuticals; OX40 modulators such as BMS-986178 from Bristol-Myers Squibb, GSK3174998 from GlaxoSmithKline, INCAGN1949 from Agenus/Incyte, MEDI0562 from MedImmune, PF-04518600 from Pfizer, or RG7888 from Genentechp; GITR modulators such as GWN323 from Novartis Pharmaceuticals, INCAGN1876 from Agenus/Incyte, MEDI1873 from MedImmune, MK-4166 from Merck, or TRX518 from Leap Therapeutics; KIR modulators such as lirilumab from Bristol-Myers Squibb; or TIM modulators such as MBG453 from Novartis Pharmaceuticals or TSR-022 from Tesaro.

In some instances, the additional therapeutic agent comprises a hormone-based therapeutic agent. Exemplary hormone-based therapeutic agents include, but are not limited to, aromatase inhibitors such as letrozole, anastrozole, exemestane, or aminoglutethimide; gonadotropin-releasing hormone (GnRH) analogues such as leuprorelin or goserelin; selective estrogen receptor modulators (SERMs) such as tamoxifen, raloxifene, toremifene, or fulvestrant; antiandrogens such as flutamide or bicalutamide; progestogens such as megestrol acetate or medroxyprogesterone acetate; androgens such as fluoxymesterone; estrogens such as estrogen diethylstilbestrol (DES), Estrace, or polyestradiol phosphate; or somatostatin analogs such as octreotide.

In some instances, the additional therapeutic agent is a first-line therapeutic agent.

In some embodiments, the anti-CLDN18.2 antibody and the additional therapeutic agent are administered simultaneously.

In some instances, the anti-CLDN18.2 antibody and the additional therapeutic agent are administered sequentially. In such instances, the anti-CLDN18.2 antibody is administered to the subject prior to administering the additional therapeutic agent. In other instances, the anti-CLDN18.2 antibody is administered to the subject after the additional therapeutic agent is administered.

In some cases, the additional therapeutic agent and the anti-CLDN18.2 antibody are formulated as separate dosage.

In some instances, the subject has undergone surgery. In some instances, the anti-CLDN18.2 antibody and optionally the additional therapeutic agent are administered to the subject after surgery. In some cases, the anti-CLDN18.2 antibody and optionally the additional therapeutic agent are administered to the subject prior to surgery.

In some instances, the subject has undergone radiation. In some instances, the anti-CLDN18.2 antibody and optionally the additional therapeutic agent are administered to the subject during or after radiation treatment. In some cases, the anti-CLDN18.2 antibody and optionally the additional therapeutic agent are administered to the subject prior to undergoing radiation.

In some instances, the subject is a human.

In some embodiments, also described herein is a method of inducing cell kill effect. In some cases, the method comprises contacting a plurality of cells with an anti-CLDN18.2 antibody comprising a payload for a time sufficient to internalize the anti-CLDN18.2 antibody to induce the cell kill effect. In some cases, the payload comprises a maytansinoid, an auristatin, a taxoid, a calicheamicins, a duocarmycin, an amatoxin, or a derivative thereof. In some cases, the payload comprises an auristatin or its derivative thereof. In some cases, the payload is monomethyl auristatin E (MMAE). In some cases, the payload is monomethyl auristatin F (MMAF).

In some instances, the cell is a cancer cell. In some cases, the cell is from a gastrointestinal cancer. In some cases, the gastrointestinal cancer is a gastric cancer. In some cases, the gastrointestinal cancer is a pancreatic cancer. In some cases, the gastrointestinal cancer is an esophageal cancer or cholangiocarcinoma. In some cases, the cell is from a lung cancer or an ovarian cancer.

In some embodiments, the method is an in vitro method.

In some embodiments, the method is an in vivo method

### Pharmaceutical Compositions

In some embodiments, an anti-CLDN18.2 antibody is further formulated as a pharmaceutical composition. In some instances, the pharmaceutical composition is formulated for administration to a subject by multiple administration routes, including but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition describe herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. Exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle. In some cases, a nanoparticle has at least one dimension of less than about 500 nm, 400 nm, 300 nm, 200 nm, or 100 nm.

In some embodiments, the pharmaceutical compositions include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical compositions include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical compositions include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical compositions described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™ Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants are included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the improved disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-CLDN18.2 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL” Generally, the term “about” includes an amount that would be expected to be within experimental error, e.g., within 15%, 10%, or 5%.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

### EXAMPLES

These examples are provided for illustrative purposes only and not to limit the scope of the claims provided herein.

### Example 1—Targets and Reagents

HEK293 and CHO cells over-expressing CLDN18.2 were generated in NovoBioSci and Genomeditech for immunization and screening purposes. HEK293 cell expressing CLDN18.2 were co-expressed with GFP by an IRES. The expression of GFP and CLDN18.2 were demonstrated by staining with a commercial available fluorescence-labeled antibody against CLDN18 (ab203563) (FIG. 1). The expression of CLDN18.2 on CHO cells was confirmed by DNA sequencing.

### Example 2—Immunization

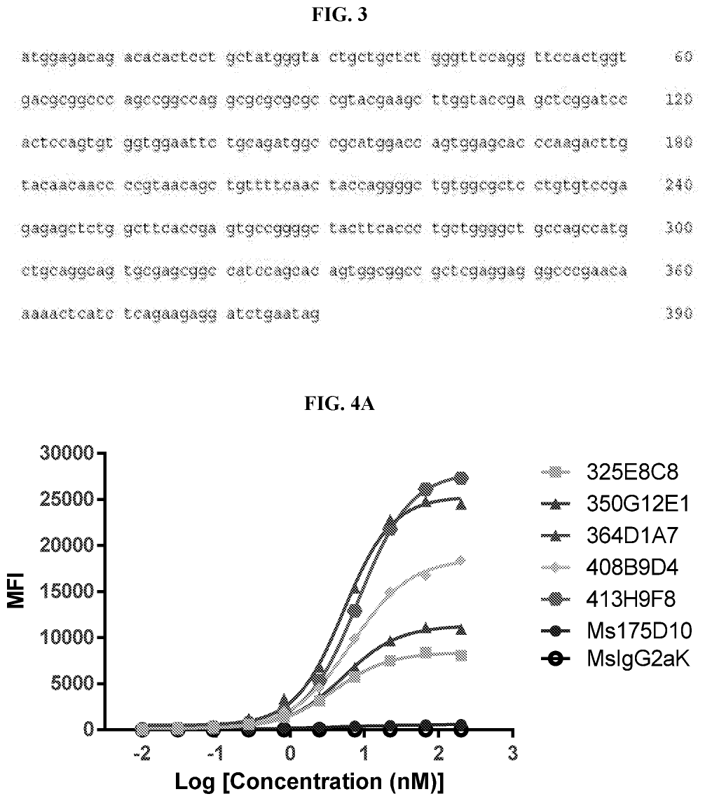

The immunization of rat to generate antibody was conducted using the following immunization schedule (Table 10). In brief, in the first two immunizations, two types of DNA constructs, either extracellular loops 1 (ECL1, FIG. 3) only or full length CLDN18.2 (FIG. 2) DNA was used. The third immunization was done with HEK293 cells over-expressing CLDN18.2, and the fourth immunization was done using either corresponding DNA or DNA with cells over-expressing CLDN18.2. The final boost was done with HEK293 cells over-expressing CLDN18.2. Four rats were used for fusion.

TABLE 10
Rat immunization scheme.

| Immunization | Antigens | | | |
|---|---|---|---|---|
| 1^{st} & 2^{nd} | CLDN18.2 - ECL1 DNA | | CLDN18.2 - FL DNA | |
| 3^{rd} | HEK293-CLDN18.2 cells | | HEK293-CLDN18.2 cells | |
| 4^{th} | CLDN18.2- | CLDN18.2- | CLDN18.2-FL | CLDN18.2-FL |
| | ECL1 DNA | ECL1 DNA + | DNA | DNA + |
| | | HEK293- | | HEK293- |
| | | CLDN18.2 cells | | CLDN18.2 cells |
| Final Boost | HEK293-CLDN18.2 cells | | | |
| Animal # | 4518* | 4521* | 4468 | 4515 |
| | 4519* | 4522 | 4472 | 4516 |
| | 4520* | 4523 | 4473 | 4517 |

*rat selected for fusion

For mouse immunization (Table 11), either CHO or HEK293 cells over-expressing CLDN18.2 were used in 4 rounds of immunization plus final boost. Four mice were used in each group and 3 fusions were performed to generate hybridomas.

TABLE 11
Mouse immunization scheme.

| Immunization | Antigens | |
|---|---|---|
| 1^{st} - 4^{th} | HEK293-CLDN18.2 cells | CHO-CLDN18.2 cells |
| Boost | HEK293-CLDN18.2 cells | CHO-CLDN18.2 cells |
| Animal # | 6375 | 6379 |
| | 6376* | 6380 |
| | 6377 | 6381 |
| | 6378* | 6382* |

*mouse selected for fusion

### Example 3—Screening of Primary Hybridoma Clones by FACS Binding

Hybridoma supernatants specifically bound to CHO-CLDN18.2. A total of 80 96-well plates were seeded and screened by cell-based ELISA from the hybridoma of immunized animals. 194 clones were identified as positive based on an OD value of >0.3. To obtain antibodies that specifically bound to CLDN18.2 but not CLDN 18.1, hybridomas from rat or/and mouse immunization with engineered cell lines, CHO-CLDN18.1 and CHO-CLDN18.2, were screened. Briefly, 50 μL CHO-CLDN18.1 or CHO-CLDN18.2 cells (cell density: 2×106 cells/mL, viability>90%) were incubated with equal volume hybridoma supernatant in 96 well plate at 4° C. for 1 h. After washed with FACS buffer (DPBS containing 2% FBS), the cells/antibody mixture was stained with secondary antibody (Goat anti Rat IgG(H+L) iFlour 647, Genscript, or Alexa Flour® 647-conjugated rabbit anti-mouse IgG, Jackson ImmunoResearch). Finally, the mixture was washed and resuspended with FACS buffer, and subjected to FACS analysis on BD FACS Celesta. The raw data was analyzed with FlowJo software.

7 hybridomas from immunized rats and 31 hybridomas from immunized mice showed stronger specific binding to CHO-CLDN18.2 when compared to their respective binding to CHO-CLDN18.1 cells.

### Example 4—Purified Antibodies Specifically Bind to CHO-CLDN18.2

Purified antibodies were generated by protein G affinity purification from the supernatants. Briefly, hybridoma supernatant was centrifugated at 8000 rpm and 4° C. for 30 minutes. Next the supernatant was filtered with 0.22 μm microfiltration membrane. NaCl was added to the supernatant at the ratio of 1 g NaCl for 10 mL supernatant. The supernatant sample was loaded on to the purification column at a velocity of 3 mL per minute at 4° C. Protein G resin was equilibrated with 4-5 column volume of 1×PBS, then washed with eluate buffer (0.1M Tris, pH12). Neutralization buffer was then immediately added to the collection tube containing the eluted antibody to neutralize the pH. Next, the eluted antibody was dialyzed against 1×PBS at room temperature for 2 hours. The antibody was subsequently stored for analysis.

Purified rat antibodies were tested in binding assay using cells over-expressing CLDN18.2 or CLDN18.1 according to the method described above. 4 purified rat antibodies 181B7B7, 193H11D8, 184A10D8, and 282A12F3 showed specific binding to CLDN18.2, but not to CLDN 18.1. Particularly, 282A12F3 showed stronger binding to CLDN18.2 than reference antibody 175D10, and two purified rat antibodies 101C6A8 and 186A4B9 bound to both CLDN18.1 and CLDN18.2.

18 purified mouse antibodies including 325F12H3, 325E8C8, 328G2C4, 350G12E1, 357B8F8, 360F1G1, 364D1A7, 382A11H12, 399H6A10, 406D10H7, 408B9D4, 409E2C5, 413B5B4, 413H9F8, 416E8G10, 417H3B1, 420G5E2, and 429G1B7 showed specific binding to CLDN18.2, but not to CLDN18.1. Particularly, 325E8C8, 350G12E1, 357B8F8, 364D1A7, 408B9D4, and 413H9F8 showed stronger binding to CHO-CLDN18.2 than reference antibody 175D10.

### Example 5—Binding Curve of Purified Antibodies

Binding curves were generated to rank the binding affinities of hybridoma antibodies. Briefly, a total of 1×10⁵ CHO-CLDN18.2 cells for each well were seeded in 96-well plate and washed by FACS buffer (DPBS containing 2% FBS) twice. Cells were incubated by series diluted purified hybridoma antibodies for 1 h. After primary antibody incubation, cells were washed by FACS buffer for two times. Then, cells were stained with secondary antibody (Alexa Flour® 647-conjugated rabbit anti-mouse IgG, Jackson ImmunoResearch). Alexa Fluor 647 signals of the stained cells were detected by BD FACS Celesta and the geometric mean fluorescence signals were determined. FlowJo software was used for analysis. Data was plotted as the logarithm of antibody concentration versus mean fluorescence signals. Nonlinear regression analysis was performed by GraphPad Prism 6 (GraphPad Software) and EC50 values were calculated.

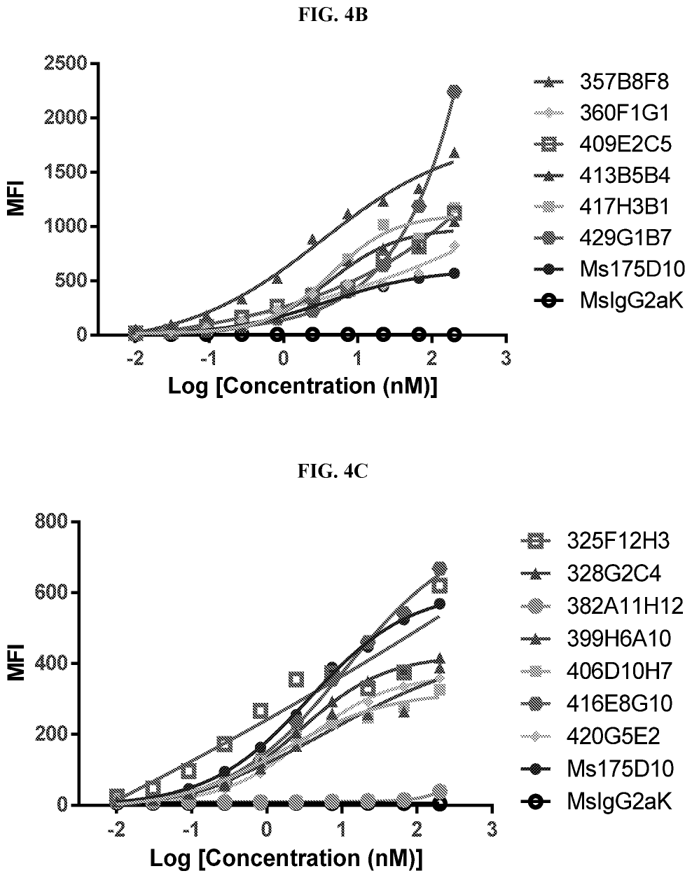

As shown in FIG. 4A-FIG. 4C, purified anti-CLDN18.2 mouse-generated antibodies showed a dose-dependent binding on CHO-CLDN18.2 cells. Five antibodies, 325E8C8, 350G12E1, 364D1A7, 408B9D4, and 413H9F8, out of a total of 18 tested antibodies showed the highest maximal binding compared with that of reference antibody 175D10 (FIG. 4A). Five antibodies, 417H3B1, 413B5B4, 357B8F8, 360F1G1 and 429G1B7 showed higher maximal binding than that of 175D10 but lower than antibodies 325E8C8, 350G12E1, 364D1A7, 408B9D4, and 413H9F8 (FIG. 4B). Additional tested antibodies showed similar or weaker maximal binding compared to that of 175D10 (FIG. 4C). The EC50s of select anti-CLDN18.2 antibodies to CLDN18.2 were about 10 nM or less (Table 12).

TABLE 12
Binding affinities (EC₅₀ₛ) to CHO-CLDN18.2 cells of antibodies
derived from mouse-immunized hybridoma clones.

| Antibodies | EC₅₀, nM |
|---|---|
| 325E8C8 | 3.86 |
| 325F12H3 | na. |
| 328G2C4 | 2.79 |
| 350G12E1 | 5.17 |
| 357B8F8 | 3.26 |
| 360F1G1 | na. |
| 364D1A7 | 5.22 |
| 382A11H12 | na. |
| 399H6A10 | 6.71 |
| 406D10H7 | 1.64 |
| 408B9D4 | 6.73 |
| 409E2C5 | na. |
| 413B5B4 | 4.07 |
| 413H9F8 | 8.30 |
| 416E8G10 | 10.97 |
| 417H3B1 | 4.34 |
| 420G5E2 | 3.24 |
| 429G1B7 | na. |
| Ms175D10 | 3.68 |
| MsIgG2aK | na. |

The term “na.” used herein and in the following tables indicates “not applicable”.

### Example 6—Binding of Antibodies to Gastric Cancer Cell Lines

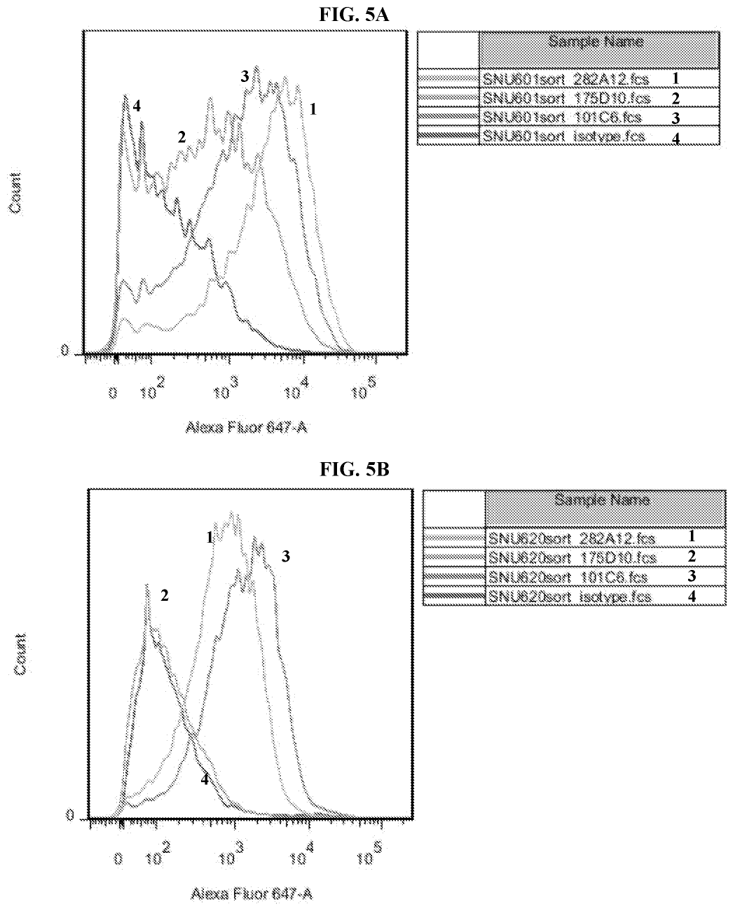

Gastric cancer cell lines SNU601 and SNU620 have endogenous expression of CLDN18.2. The expression of CLDN18.2 on SNU601 and SNU620 cells were confirmed by RT-PCR using CLDN18.2 specific primers and DNA sequencing. SNU601 and SNU620 cells with high level expression of CLDN18.2 were sorted for binding assay. Binding assay was performed as described previously. Rat generated clones 282A12 and 10106 and the reference antibody 175D10 all bound to gastric cancer line SNU601, but clones 282A12 and 10106 also bound to SU620 (FIG. 5A and FIG. 5B).

SNU620 cell line was used for determining binding affinities of mouse monoclonal antibodies to endogenous expressed CLDN18.2. All murine-immunized positive antibodies were tested at the final concentration of 10 μg/mL. 15 out of the 18 mouse monoclonal antibodies including 325F12H3, 325E8C8, 328G2C4, 350G12E1, 360F1G1, 364D1A7, 406D10H7, 408B9D4, 409E2C5, 413B5B4, 413H9F8, 416E8G10, 417H3B1, 420G5E2, and 429G1B7 showed stronger binding to SU620 compared with 175D10. Particularly, 413H9F8, 364D1A7, and 408B9D4 bound to SNU620 cancer cells strongly.

In summary, antibodies such as 282A12F3, 364D1A7, and 413H9F8 bound to CHO-CLDN18.2 and gastric cancer SNU620 specifically (Table 13).

TABLE 13
Summary of binding activities of CLDN18.2-specific antibodies.

| | CHO-18.1 | CHO-18.2 | SNU620 | |
|---|---|---|---|---|
| | (@30 ug/ml) | (@30 ug/ml) | (@10 ug/ml) | Isotype |
| 175D10 | − | ++ | − | IgG |
| 282A12 | − | +++ | ++ | IgG |
| 325F12H3 | − | ++ | + | IgG |
| 328G2C4 | − | + | + | IgG |
| 360F1G1 | + | ++ | + | IgG |
| 382A11H12 | na | na | na | IgM |
| 399H6A10 | + | ++ | + | IgG |
| 406D10H7 | − | + | + | IgG |
| 420G5E2 | − | + | + | IgG |
| 429G1B7 | + | +++ | + | IgG |
| 409E2C5 | − | +++ | + | IgG |
| 413B5B4 | − | +++ | + | IgG |
| 416E8G10 | + | ++ | + | IgG |
| 417H3B1 | − | +++ | + | IgG |
| 325E8C8 | − | +++ | + | IgG |
| 350G12E1 | − | +++ | + | IgG |
| 357B8F8 | − | +++ | − | IgG |
| 364D1A7 | − | +++ | ++ | IgG |
| 408B9D4 | − | +++ | ++ | IgG |
| 413H9F8 | − | +++ | ++ | IgG |

### Example 7—Chimerization

Murine and rat antibodies were chimerized by expressing murine and rat light chain variable region in the pCDNA3.1(+) plasmid which comprises a DNA sequence encoding amino acids of a signal sequence and a constant region of human IgG1. The sequences of heavy and light chain constant regions (CH and CL) of human IgG1 are shown in Table 4.

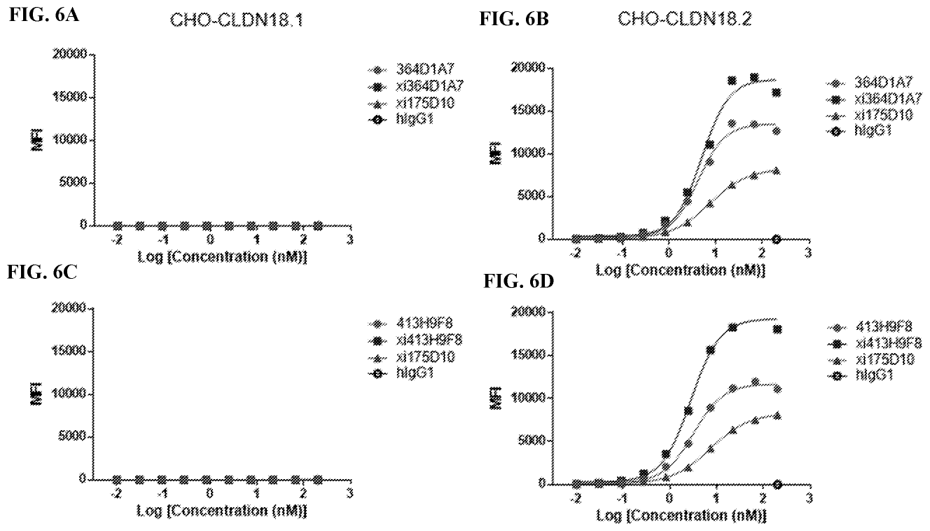

The binding affinities of chimeric antibodies on CHO-CLDN18.2 cell line were determined as described previously. As shown in FIG. 6A-FIG. 6D, chimeric antibodies 282A12F3, 64D1A7, and 413H9F8 specifically bind to CLDN18.2. Chimeric 282A12F3, 64D1A7 and 413H9F8 showed stronger binding affinities as compared with reference antibody 175D10.

### Example 8—Antibody Sequence Analysis and Removal of Post-Translational Modification Sites

The sequences of antibodies produced by hybridoma technology were analyzed for post-translational modifications (PTMs), which sometimes cause problems during the development of a therapeutic protein such as increased heterogeneity, reduced bioactivity, reduced stability, immunogenicity, fragmentation and aggregation. The potential impact of PTMs depends on their location and in some cases on solvent exposure. The CDRs of all sequences were analyzed for asparagine deamination, aspartate isomerization, free cysteine thiol groups, N-glycosylation, oxidation, and fragmentation by potential hydrolysis sites.

Multiple alignments of the parental sequences to the human germline sequences were performed using Igblast tool. Based on the parental antibody sequence alignment to the human germlines, the highest homology entries were identified.

Structural models of antibody 282A12F3, 413H9F8 and 364D1A7 were generated using customized Build Homology Models protocol. Candidate structural template fragments were scored, ranked and selected from PDB database based on their sequence identity to the target, as well as qualitative crystallographic measures of the template structure. Based on the homology modelling data for 282A12F3, 413H9F8, 364D1A7 individually, exposed residues in the framework region (FR) and CDR regions were identified, potential PTM sites on protein structure surface were highlighted. Base on the PTM analysis data and sequence identity for human germline template, three antibodies 282A12F3, 413H9F8, and 364D1A7 were utilized as parental antibodies for further humanization.

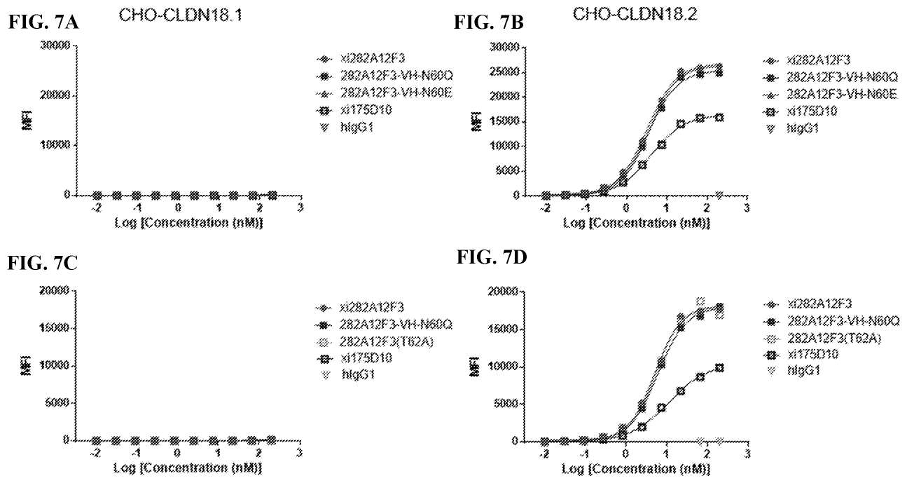

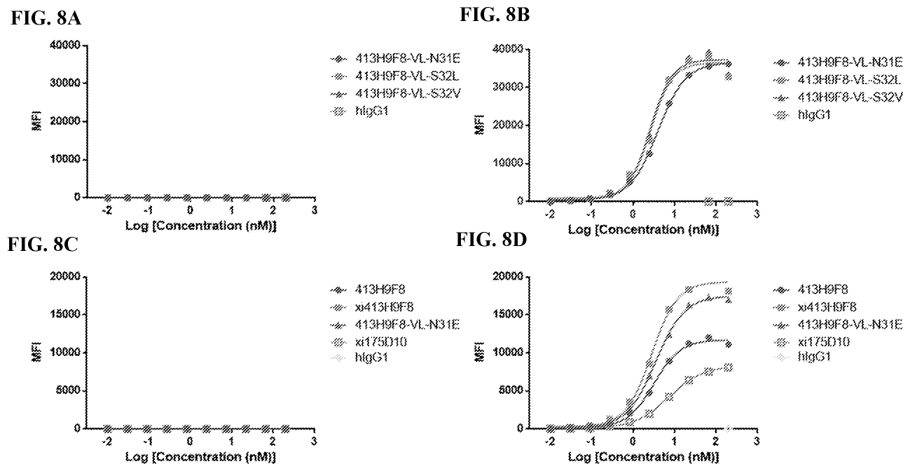

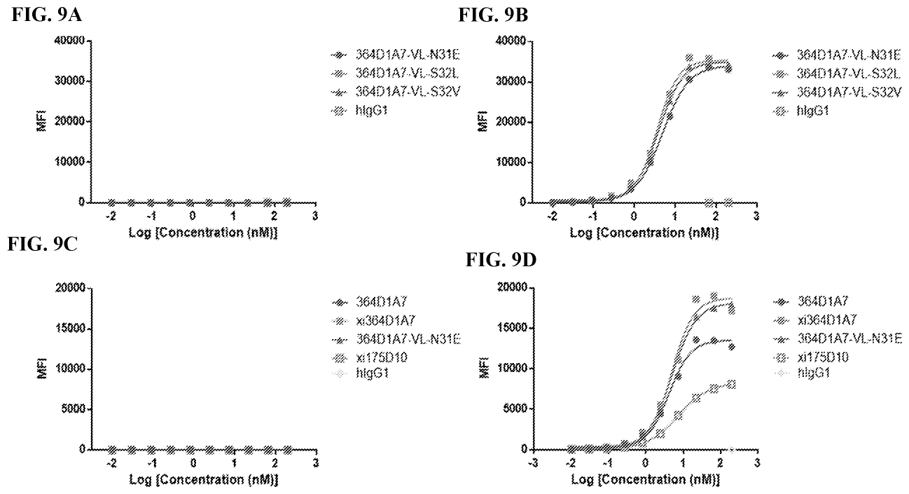

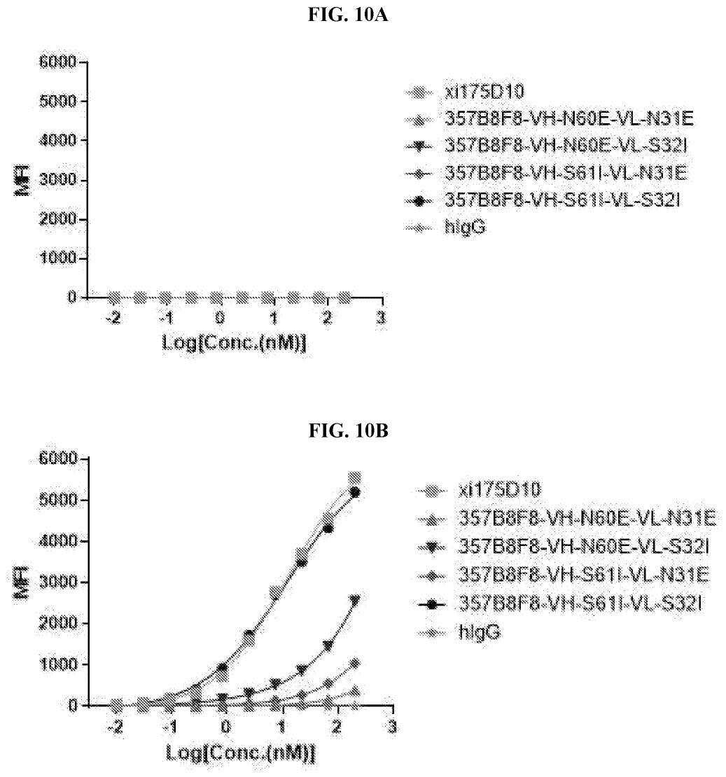

Binding test of PTM sites removed mutants were tested on cells expressing CLDN18.2 or CLDN18.1, along with reference antibody 175D10 as positive control. As shown in FIG. 7A-FIG. 10B, after potential PTM site removal, 282A12F3-VH-N60Q, 282A12F3-VH-N60E, and 282A12F3(T62A) from 282A12F3 clone, 413H9F8-VL-N31E, 413H9F8-VL-S32L, and 413H9F8-VL-S32V from 413H9F8 clone, and 364D1A7-VL-N31E, 364D1A7-VL-S32L, and 364D1A7-VL-S32V from 364D1A7 clone showed specific binding to CHO-CLDN18.2 instead of CHO-CLDN18.1 cell lines. All of the antibodies with potential PTM site removal had stronger binding compared with reference antibody 175D10 to CHO-CLDN18.2 cells. 357B8F8-VH-N60E-VL-N31E, 357B8F8-VH-N60E-VL-S32I, 357B8F8-VH-S61I-VL-N31E, and 357B8F8-VH-S61I-VL-S32I from 357B8F8 clone showed specific binding to CHO-CLDN18.2, however only 357B8F8-VH-S61I-VL-S32I showed comparable binding affinity with 175D10 (xi175D10) to CHO-CLDN18.2.

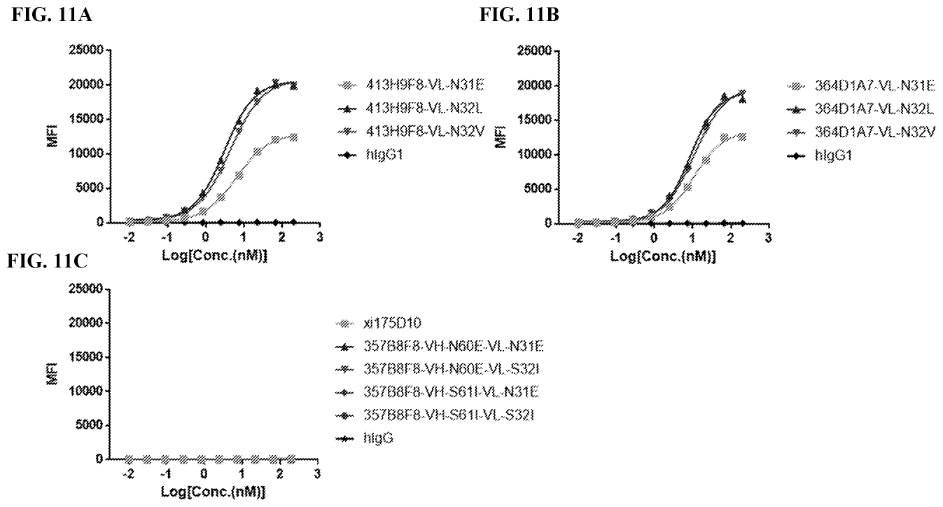

The chimeric clones with PTM removal mutations were tested for their binding to SNU620 with endogenous CLDN18.2 expression as described above. As shown in FIG. 11A-FIG. 11C, both 413H9F8 and 364D1A7 variants bound to SNU620 at different levels. The S32V and S32L mutants showed better binding activity to CHO-CLDN18.2 and SNU620 cells than the N31E mutant. Clone 413H9F8 showed better binding activity to CLDN18.2 than 364D1A7. Chimeric 357B8F8, and its PTM removal variants, could not bind to SNU620 cancer cell line, which is similar to the reference antibody 175D10.

### Example 9—Competitive Binding of Chimeric Antibodies

To investigate the epitope binding group of CLDN18.2-binding antibodies, four chimeric antibodies were tested for their competitive binding activities using CHO-CLDN18.2 cells. The working concentration of each antibody was determined by CHO-CLDN18.2 cell-based binding assay. Cells were collected and washed with PBS, then 1×10⁵ cells in 50 μL in PBS were added in 96-well plate. Test antibodies were diluted from 60 μg/mL with PBS in 3-fold series for 12 points and 50 μL of diluted antibodies were mixed with cells, and incubated at 4° C. for 120 min. Next, the wells were washed with PBS. For competitive binding assays, 100 μL biotin-labeled anti-CLDN18.2 antibodies was added at working concentration (5, 1, 0.5 and 1 μg/mL of xi175D10, 282A12F3 (T62A), 413H9F8-VL-S32V, and 364D1A7-VL-S32V respectively). Biotin-labeled goat anti-human IgG Fc was added in 1:800 dilution at 100 μL/well as the control. The plates were incubated at 4° C. for 40 min. Streptavidin-APC (1:1700) was used to detect biotin-labeled antibody. Flow cytometry was performed to measure the binding.

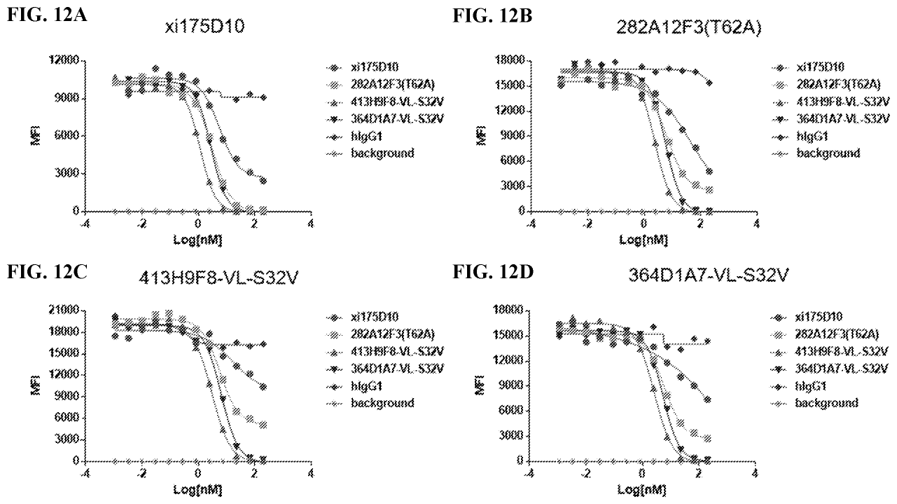

Binding of 175D10 on CHO-CLDN18.2 was completely inhibited by 282A12F3 (T62A), 413H9F8-VL-S32V, or 364D1A7-VL-32V (FIG. 12A-FIG. 12D). Binding of 282A12 (T62A) on CHO-CLDN18.2 was completely inhibited by 413H9F8-VL-532V or 364D1A7-VL-S32V, partially inhibited by 175D10. Binding of 413H9F8-VL-S32V and 364D1A7-VL-S32V on CHO-CLDN18.2 was partially inhibited by 175D10 or 282A12F3 (T62A).

### Example 10—Cross-Binding Activity on Mouse and Cynomolgus CLDN18.2

Species cross-reactivity enables evaluation of the clinical candidate in pharmacology models (mice) and toxicity models (cynomolgus monkey). The species cross-reactivity of anti-human CLDN18.2 antibodies were determined by cell based binding assay. Binding of the identified monoclonal antibodies to murine and cynomolgus CLDN18.2 was analyzed by flow cytometry. HEK293 cells were transiently co-transfected with a fluorescence marker and murine CLDN18.2 and cynomolgus CLDN18.2. Briefly, 2.5×10⁶ HEK-293 cells per dish were plated into two 10-cm² dishes with 10 mL DMEM medium for each dish. 24 h after planting, cells were transfected with mouse GFP-CLDN18.2 and cynomolgus GFP-CLDN18.2 plasmids. A total plasmid mass of 10 μg per dish was transfected using 20 μL Lipofectamine 2000 (Life Technologies). Culture medium was replaced 5 h after transfection. 48 h after transfection, cells were dissociated and prepared for binding affinity detection. The stable cell line HEK293-GFP-CLDN18.2, which expressing human CLDN18.2, was used to detect human GFP-CLDN18.2.

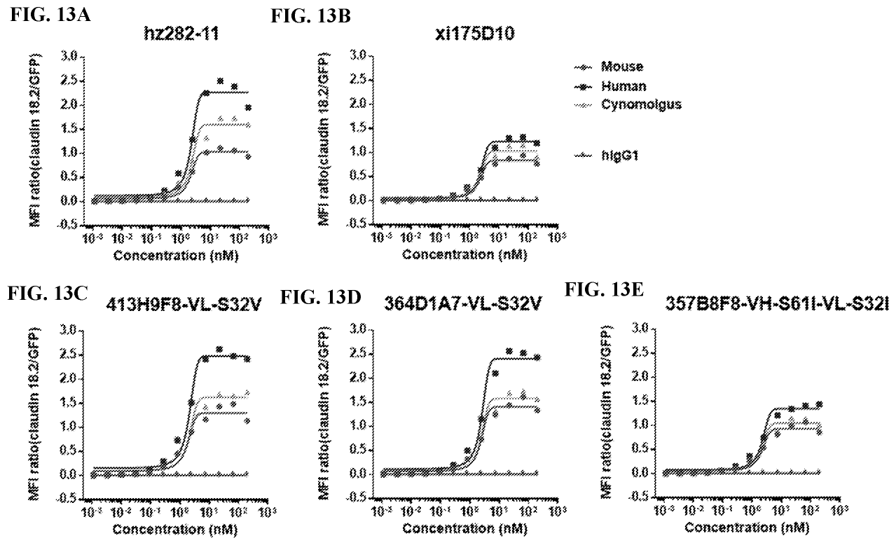

The binding affinities of anti-human CLDN18.2 antibodies were determined on human, mouse and cynomolgus CLDN18.2 over-expressing cells. Briefly, 1×10⁵ cells for each well were seeded in 96-well plate and washed by FACS buffer (D-PBS containing 2% FBS) for two times. Cells were incubated by series diluted anti-CLDN18.2 antibodies for 1 h. Control group comprised cancer cells incubated with human IgG1. After primary antibody incubation, cells were washed by FACS buffer for two times. Then, cells were stained by Alexa Fluor 647 labeled anti-human IgG secondary antibody (Jackson ImmunoResearch Laboratories) for 30 min at 4° C. Alexa Fluor 647 and GFP signals of the stained cells were detected by BD FACS Celesta and the geometric mean fluorescence signals were determined. FlowJo software was used for analysis. Data was plotted as the antibody concentration versus mean fluorescence ratio by Alexa Fluor 647/GFP. Nonlinear regression analysis was performed by GraphPad Prism 6 (GraphPad Software). As shown in FIG. 13A-FIG. 13E, variants of chimeric antibodies 413H9F8, 364D1A7, and 357B8F8, humanized 282A12(T62A) (hz282-11) and reference antibody 175D10 cross reacted with mouse and cynomolgus CLDN18.2. All of the tested antibodies showed stronger binding to human CLDN18.2 compared to those of cynomolgus and mouse CLDN18.2.

### Example 11—Antibody-Dependent Cellular Cytotoxicity (ADCC) of Chimeric Antibodies

Chimeric Anti-CLDN18.2 Antibodies Induced Specific ADCC on CHO-CLDN18.2 Cells.

The specificity of anti-CLDN18.2 antibodies induced ADCC was tested on CHO-CLDN18.1 and CHO-CLDN18.2 cells. The target cells, CHO-CLDN18.1 and CHO-CLDN18.2, were labeled by CFSE (Life technology) at a final concentration of 2.5 μM for 30 min. Labeled target cell concentration was adjusted to 2×10⁵ cells/mL, effector cells (FcR-TANK (CD16A-15V), which was an engineered NK92 cell line overexpressing CD16a developed by ImmuneOnco) were adjusted to 8×10⁵ cells/mL. Then, 50 μL of target cell suspension, 100 μL of effector cell suspension and 50 μL of series diluted antibodies were mixed in each well (effector cell/target cell ratio was 8:1). Duplicate wells were prepared for each concentration of antibody. Control group comprised cancer cells incubated only with effector cells. After incubation at 37° C., 5% CO₂ for 4-16 h, 1 μg/mL 7-AAD (Invitrogen) was added and analyzed by flow cytometry (BD FACS Celesta). ADCC was calculated by the formula: ADCC %=% 7-AAD positive cell with antibody-% 7-AAD positive cell without antibody.

Anti-CLDN18.2 Antibodies and FcR-TANK (CD16A-15V) Cell Induced ADCC on NCI-N87-CLDN18.2 Gastric Cell Line

The ADCC of chimeric antibodies and humanized antibodies were tested on NCI-N87 cancer cells. The target cells were labeled by CFSE (Life technology) at the final concentration of 2.5 μM for 30 min. Labeled target cell concentration was adjusted to 2×10⁵ cells/mL, effector cells (FcR-TANK (CD16A-15V) were adjusted to 8×10⁵ cells/mL. Then, 50 μL of target cell suspension, 100 μL of effector cell suspension and 50 μL of series diluted antibodies were mixture in each well (effector cell/target cell ratio was 8:1). Duplicate wells were prepared for each concentration of antibody. Control group comprised cancer cells incubated only with effector cells. After incubation at 37° C., 5% CO₂ for 4-16 h, 1 μg/mL 7-AAD (Invitrogen) was added and analyzed by flow cytometry (BD FACS Celesta). ADCC was calculated by the formula: ADCC %=% 7-AAD positive cell with antibody-% 7-AAD positive cell without antibody.

Human Peripheral Blood Mononuclear Cell (PBMC) Induced ADCC on NUGC4-CLDN18.2 Gastric Cancer Cell Line

The ADCC of chimeric antibodies and humanized antibodies induced by PBMCs were tested on NUGC4-CLDN18.2 gastric cancer cells. Cryopreserved PBMCs (AllCells) of a healthy subject were thawed one day before the assay and cultured overnight in RPMI-10% FBS medium with 200 IU IL-2 (R&D) in a CO₂ incubator. The target cells were labeled by CFSE (Life technology) at the final concentration of 2.5 μM for 15 min. After staining, cell concentration was adjusted to 6×10⁴ cells/mL and mixed with 2-times volume of PBMCs which were adjusted to 1×10⁶ cells/mL (effector cell/target cell ratio was 40:1). Then, 150 μL of mixed target and effector cell suspension and 50 μL of series diluted antibodies were mixed in each well. Duplicate wells were prepared for each concentration of antibody. Target cell alone was control group. After incubation at 37° C., 5% CO₂ for 5 h, 1 μg/mL PI (Invitrogen) was added and analyzed by flow cytometry (BD FACS Celesta). Specific cytotoxicity was calculated by the formula: specific cytotoxicity=% PI positive cell with antibody−% PI positive cell without antibody.

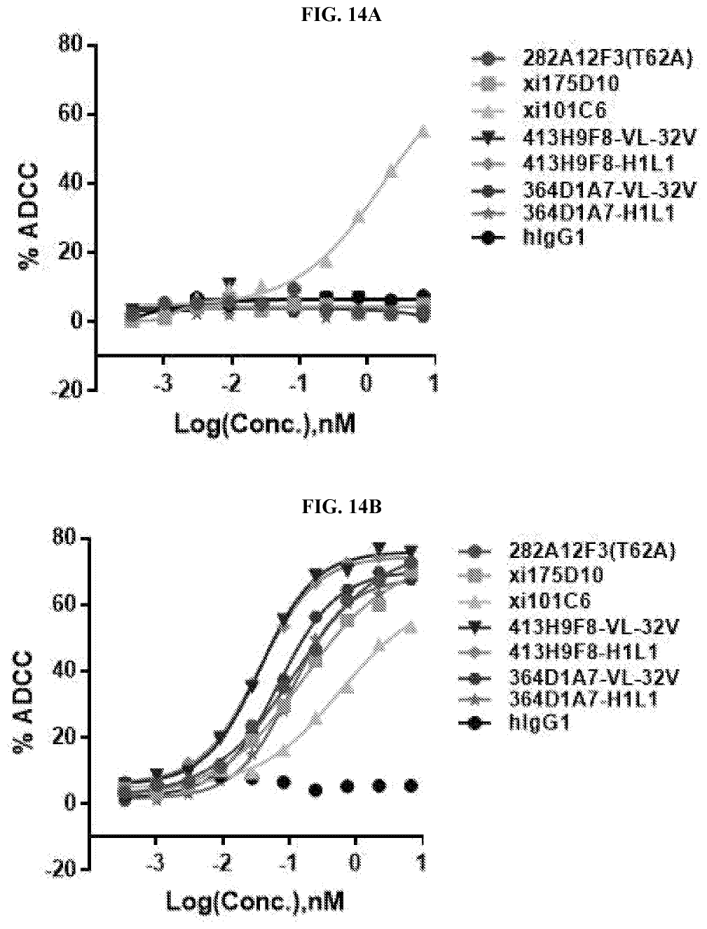

Tumor specific mAb may exert their effects through Fc-based mechanisms including antibody-dependent cell-mediated cytotoxicity (ADCC). The ADCC function of CLDN18.2 specific chimeric antibodies were analyzed by NK cell line or PBMC induced ADCC in the presence of selected antibodies. As shown in FIG. 14A-FIG. 14B, chimeric antibodies were analyzed for their capability to induce ADCC with FcR-TANK (CD16A-15V) against CHO cells with stably expression of human CLDN18.1 (CHO-CLDN18.1) or human CLDN18.2 (CHO-CLDN18.2). CLDN18.2 specific antibodies, 282A12F3 (T62A), xi175D10, 413H9F8, and 364D1A7 induced ADCC mediated lysis of CHO-CLDN18.2 but not CHO-CLDN18.1. Clone 10106, which binds to both CLDN18.1 and CLDN18.2, induced ADCC activity against both CHO-CLDN18.1 and CHO-CLDN18.2 cells. The specific ADCC activity of 282A12F3 (T62A), xi175D10, 413H9F8, and 364D1A are consistent with their specific binding profiles to CLDN18.2.

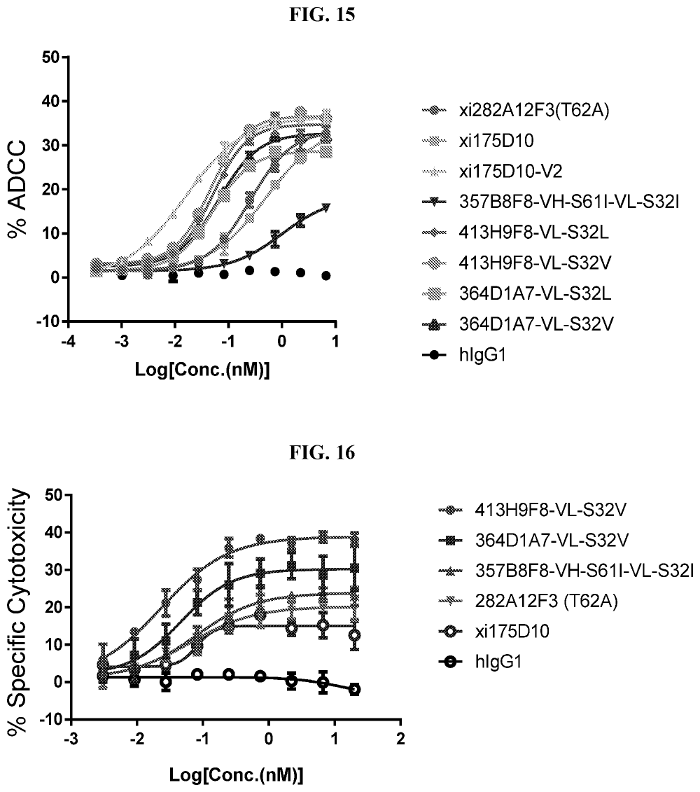

Gastric cancer line NCI-N87 with stable expression of human CLDN18.2 (NCI-N87-CLDN18.2) was used as target cell to test the ADCC activities of chimeric antibodies. As shown in FIG. 15 and Table 14, 282A12F3 (T62A), reference antibody 175D10, 413H9F8, and 364D1A7 induced ADCC mediated lysis of NCI-N87-CLDN18.2 cells. Clone 282A12F3, 413H9F8, and 364D1A7 showed stronger ADCC activity than reference antibody 175D10, while 357B8F8 showed less activity. S239D/I332E Fc variants have been shown to mediate enhanced ADCC activity of antibodies (Lazar, et al., “Engineered antibody Fc variants with enhanced effector function,” PNAS USA 2006; 103: 4005-4010). S239D/I332E mutations in Fc of 175D10 were introduced to enhance ADCC activity (175D10-V2). As shown in FIG. 15 and Table 14, 175D10 with S239D/I332E mutations in Fc (xi175D10-V2) had stronger ADCC activity than its parental antibody 175D10.

To further validate the function of CLDN18.2 specific antibodies, cryopreserved PBMCs from healthy human donors were used to test their ADCC activities against another gastric cancer line NUGC4 with stable expression of CLDN18.2 (NUGC4-CLDN18.2). As shown in FIG. 16 and Table 15, 282A12F3 (T62A), xi175D10, 413H9F8, and 364D1A7 induced ADCC mediated lysis of NUGC4-CLDN18.2 cells in a concentration dependent manner. 282A12F3, 357B8F8, 413H9F8, and 364D1A7 showed stronger ADCC activity than control antibody 175D10, and 413H9F8 showed the highest maximal specific cytotoxicity.

TABLE 14
ADCC activity of chimeric antibody on NCI-N87-18.2 cell line

| Antibody | EC₅₀, nM* |
|---|---|
| 282A12F3 (T62A) | 73.24 |
| xi175D10 | 96.43 |
| xi175D10-V2 | 3.51 |
| 357B8F8-VH-S61I-VL-S32I | 158.60 |
| 413H9F8-VL-S32L | 8.56 |
| 413H9F8-VL-S32V | 7.84 |
| 364D1A7-VL-S32L | 9.56 |
| 364D1A7-VL-S32V | 12.02 |
| hIgG1 | na. |

*Average of 2 independent experiments

TABLE 15
ADCC activity of chimeric antibodies
on NUGC4-CLDN18.2 cell line

| | | % specific cytotoxicity |
|---|---|---|
| Antibody | EC50, nM | @ 6.67 nM |
| 413H9F8-VL-S32V | 7.69 | 31.4 |
| 364D1A7-VL-S32V | 6.36 | 26.6 |
| 357B8F8-VH-S61I-VL-S32I | 4.87 | 19.6 |
| 282A12F3 (T62A) | 9.52* | 21.0 |
| xi175D10 | 9.21 | 16.8 |
| hIgG1 | na. | −0.8 |

*Data from 1 donor. Remaining data indicate an average of 3 donors.

### Example 12—Complement-Dependent Cytotoxicity (CDC) Activities of Chimeric Antibodies

In some instances, tumor specific mAbs also exert their effects through complement-dependent cytotoxicity (CDC). Human serum and CHO-CLDN18.2 cell lines were used to validate CDC function of chimeric antibodies. 50 μL 3×10⁴ CHO-CLDN18.2 cells were mixed with 25 μL serial diluted chimeric anti-human CLDN18.2 mAbs. Incubated for 1530 min at room temperature. 25 μL of 40% human serum was added to get final serum concentration of 10%. After incubation at 37° C., 5% CO₂ for 30 min, 1 μg/mL PI (Invitrogen) was added and analyzed by flow cytometry (BD FACS Celesta).

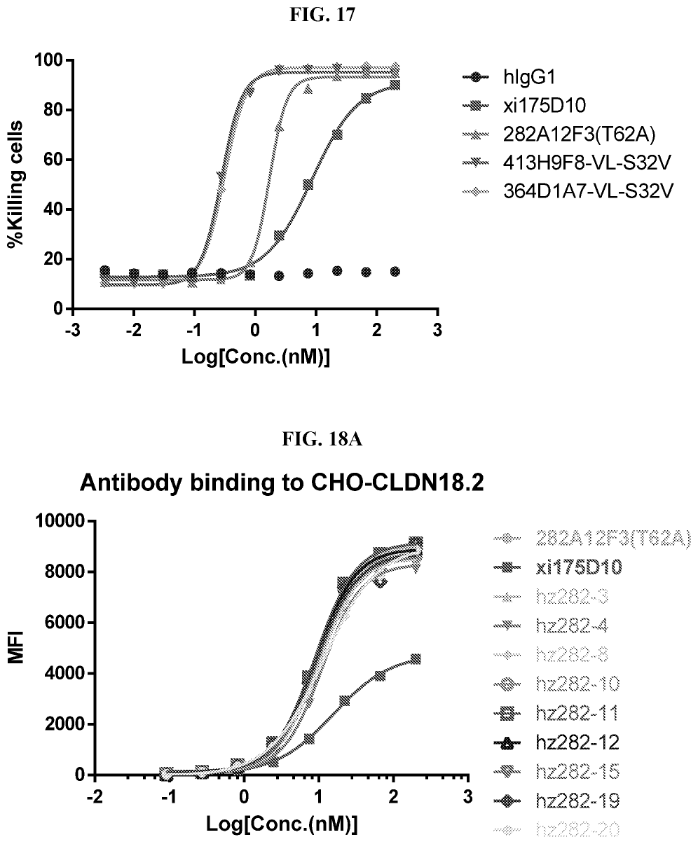

As shown in FIG. 17, chimeric antibodies, 282A12F3 (T62A), xi175D10, 413H9F8-VL-S32V, and 364D1A7-VL-S32V induced CDC mediated lysis of CHO-CLDN18.2. Antibodies 282A12F3 (T62A), 413H9F8-VL-S32V, and 364D1A7-VL-S32V induced stronger CDC compared with reference antibody 175D10.

### Example 13—Humanization of Exemplary Anti-CLDN18.2 Antibodies

Humanization of Antibody 282A12F3

Humanization of murine antibody was performed by grafting CDRs residues from mouse antibody onto a human germline framework. First, the sequences of the VH and VL region of selected candidates were compared with human germline sequences, and the best-fit germline acceptors were selected based on homology, canonical structure and physical properties. Subsequently, structure models of candidates were generated using homology modelling. The CDR regions in both heavy and light chains of candidate antibodies were fixed and the murine frameworks were replaced with selected human germline frameworks. Different residues between mouse and human frameworks that potentially influence CDR conformation were subjected to back mutation. DNA fragments encoding the designed humanized variants were synthesized and subcloned into IgG expression vectors. DNA sequences were confirmed by sequencing. Different combinations of humanized heavy and light chains were co-transfected into CHO-K1 for expression. The humanized antibodies were compared with parental antibody in antigen binding affinity, for example, by FACS on cells expressing the target antigen.

There was one glycosylation site in the VH sequence which was mutated from T to A. The sequence also did not contain free cysteine or Asn/Asp degradation motifs NG or DG. The original sequence of 282A12 VH (SEQ ID NO: 40) and 282A12 VL (SEQ ID NO: 44) was input into BLAST for analysis; and the sequence of the best mutation site was selected according to homology analysis for CDR grafting.

SEQ ID NOs: 65-68 illustrate 4 variant 282A12 VH sequences and SEQ ID NOs: 69-73 illustrate 5 variant 282A12 VL sequences.

Table 6 illustrates the humanized heavy and light chain combinations of 282A12F3 (T62A).

Humanization of Antibody 413H9F8-VL-S32V

Two strategies with a slightly different CDR-grafting approach were utilized for the humanization design of 413H9F8-VL-S32V. SEQ ID NOs: 74-76 illustrate 3 variant 413H9F8 VH sequences and SEQ ID NOs: 77-80 illustrate 4 variant 413H9F8 VL sequences that utilized a first strategy. Table 7 illustrates the humanized heavy and light chain combinations of 413H9F8-VL-S32V derivatives.

Under a second strategy, SEQ ID NOs: 81-84 illustrate 4 variant 413H9F8 VH sequences and SEQ ID NOs: 85-88 illustrate 4 variant 413H9F8 VL sequences. Table 8 illustrates the humanized heavy and light chain combinations of 413H9F8-VL-S32V derivatives.

Humanization of 364D1A7-VL-S32V

SEQ ID NOs: 89-92 illustrate 4 variant 364D1A7 VH sequences and SEQ ID NOs: 93-97 illustrate 5 variant 364D1A7 VL sequences. Table 9 illustrates the humanized heavy and light chain combinations of 364D1A7-VL-S32V derivatives.

### Example 14—Binding Activities of Humanized 282A12F3 (T62A) Antibodies

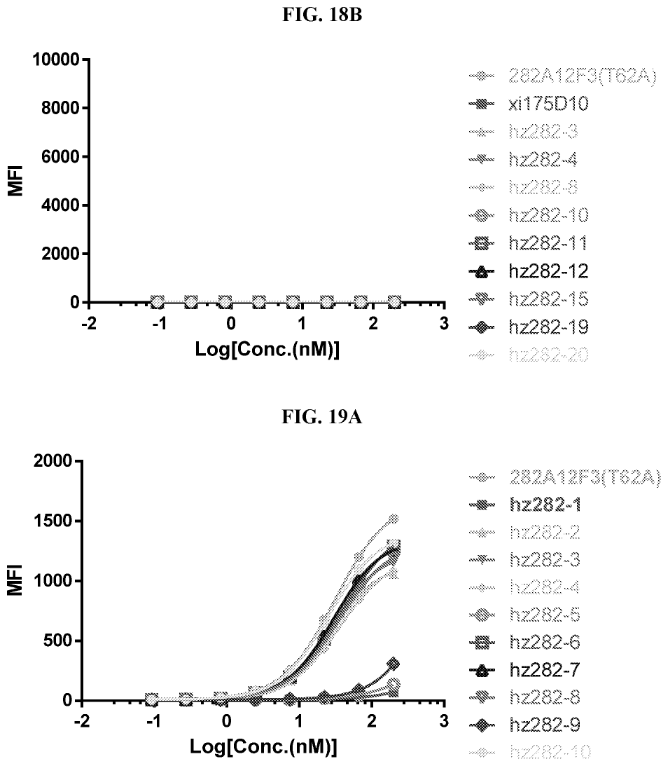

The binding affinities and specificity of humanized antibodies were compared with those of parental antibody by FACS analysis using CHO-CLDN18.1 and CHO-CLDN18.2 cells described above. As shown in FIG. 18A-FIG. 18B, humanized 282A12F3 (T62A) clones including hz282-3, hz282-4, hz282-8, hz282-10, hz282-11, hz282-12, hz282-15, hz282-19, and hz282-10 showed similar binding affinities to CHO-CLDN18.2 with 282A12F3 (T62A). None of the humanized clones bound to CHO-CLDN18.1. The data indicates that the humanized 282A12F3 (T62A) antibodies retained binding specificity and affinity to CLDN18.2.

The binding affinities of humanized 282A12T62A clones were further validated on SNU620 gastric cancer cell. As shown in FIG. 19A-FIG. 19B, the majority of the humanized 282A12T62A clones showed high binding affinities to SNU620 cancer cells. Antibodies comprising the 282A2-VHg0 heavy chains, e.g., hz282-1, hz282-5, hz282-9, hz282-13, and hz282-17, did not bind to SNU620, indicating that at least 2 residues, K and V, in the FR3 of Vh region of 282A12(T62A) are involved in binding to SNU620 (Table 6).

Binding affinity and specificity data are summarized in Table 16. Most of the humanized antibodies of 282A12 (T62A) retained the specificity and affinity of paternal antibodies.

TABLE 16
Summary of binding activities of humanized 282A12 (T62A)
antibodies on CHO-CLDN18.2 and SNU620 cancer cell line.

| | Exp. 1 | Exp. 2 | | Exp. 3 | |
|---|---|---|---|---|---|
| | MFI_CHO-18.2/CHO-8.1 | MFI_CHO-18.2/CHO- 18.1 | | MFI_SNU620 | |
| ID | (@30 ug/ml) | (@30 ug/ml) | EC50 | (@30 ug/ml) | EC50 |
| huIgGl | 1.1 | 1.0 | — | 15.7 | — |
| Xi-175D10 | 1034.6 | 1388.8 | 1.21 | 81.5 | — |
| X1-282A12 | 810.8 | 768.2 | 0.66 | 1519 | 2.88 |
| hz282-l | 261.9 | — | — | 69.6 | 48.23 |
| hz282-2 | 521.4 | — | — | 1061 | 2.56 |
| hz282-3 | 1101.6 | 1674.0 | 0.63 | 1154 | 2.78 |
| hz282-4 | 1237.7 | 2538.8 | 0.78 | 1082 | 3.35 |
| hz282-5 | 309.5 | — | — | 135 | 81.18 |
| hz282-6 | 521.3 | — | — | 1289 | 3.66 |
| hz282-7 | 623.9 | — | — | 1254 | 2.73 |
| hz282-8 | 959.5 | 1608.1 | 0.69 | 1191 | 3.33 |
| hz282-9 | 162.7 | — | — | 308 | 9773.00 |
| hz282-10 | 807.9 | 1504.9 | 0.58 | 1314 | 2.25 |
| hz282-11 | 1186.4 | 2000.7 | 0.60 | 1336 | 2.02 |
| hz282-12 | 1226.1 | 1935.4 | 0.62 | 1224 | 2.09 |
| hz282-13 | 200.8 | — | — | 144 | 35.79 |
| hz282-14 | 797.9 | — | — | 1225 | 2.72 |
| hz282-15 | 1380.5 | 2015.7 | 0.62 | 1232 | 2.24 |
| hz282-16 | 550.0 | — | — | 1278 | 3.30 |
| hz282-17 | 248.8 | — | — | 176 | 46.18 |
| hz282-18 | 781.3 | — | — | 1212 | 3.08 |
| hz282-19 | 812.5 | 1006.4 | 0.59 | 1313 | 2.29 |
| hz282-20 | 980.6 | 987.1 | 0.81 | 1236 | 2.50 |

—, Not tested or not applicable

### Example 15—Binding Activities of Humanized 413H9F8-VL-S32V and 364D1A7-VL-S32V Antibodies

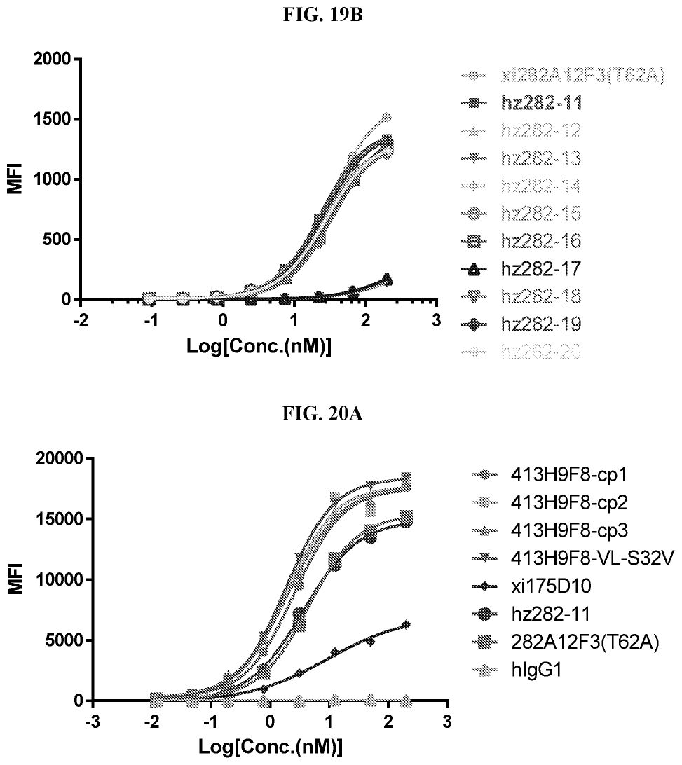

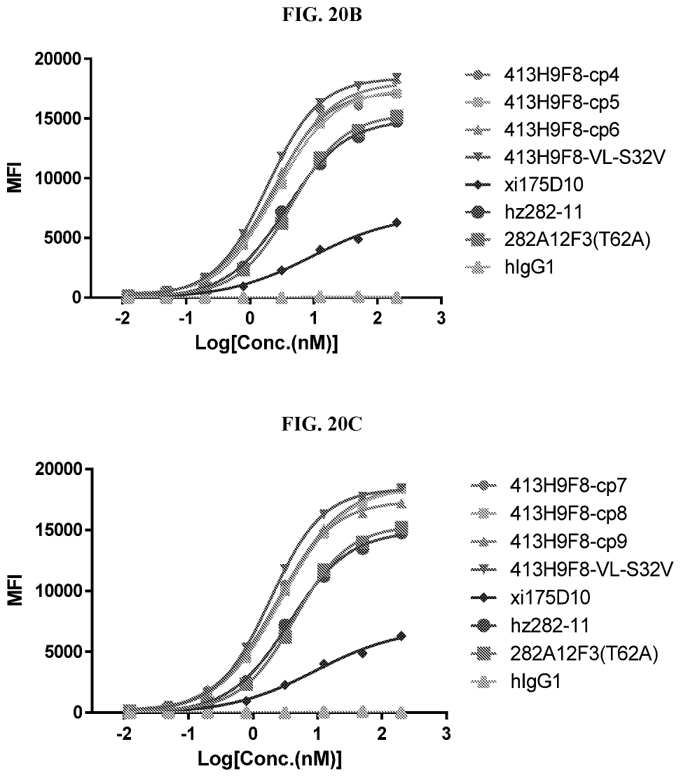

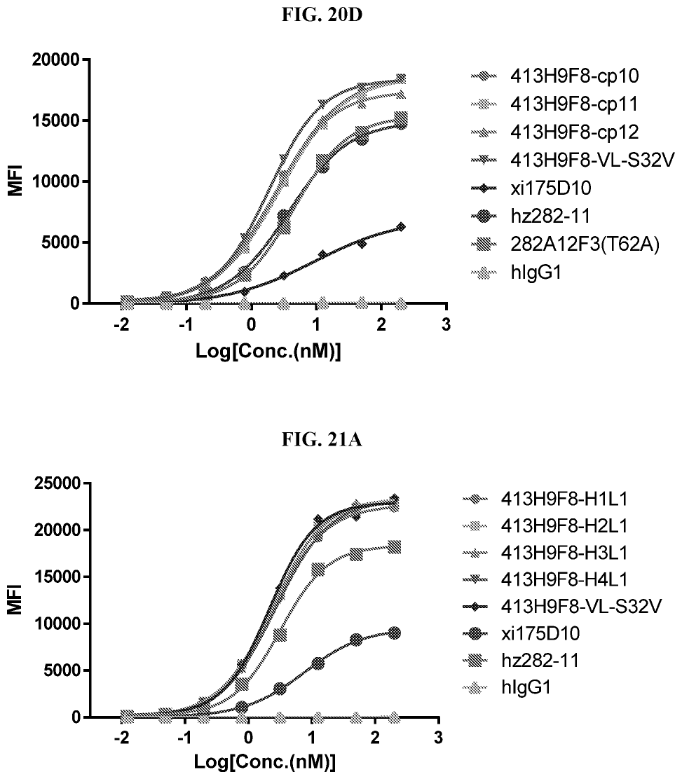

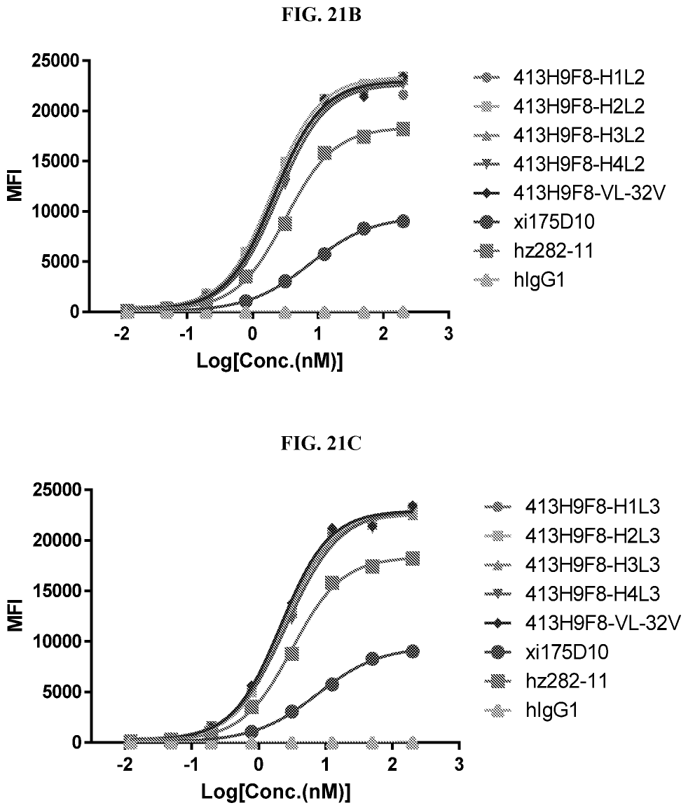

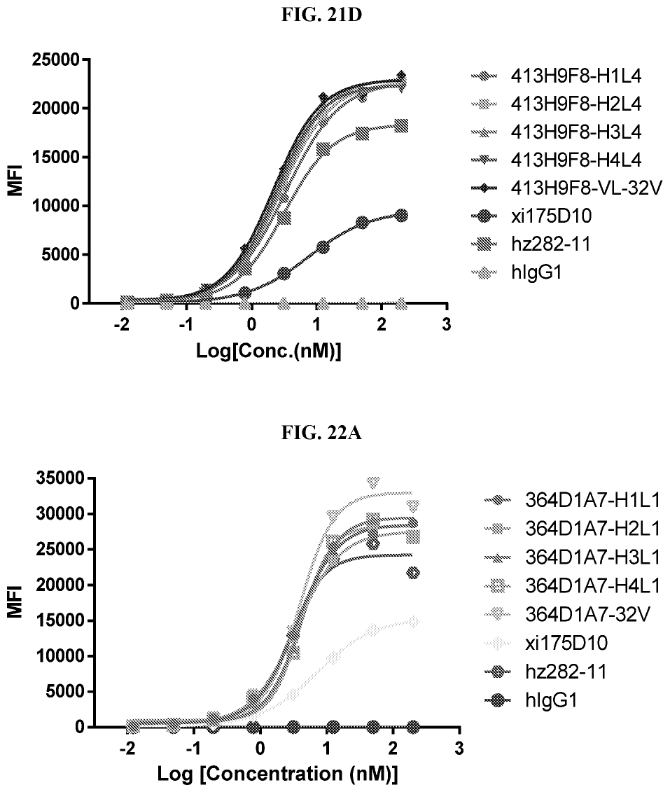

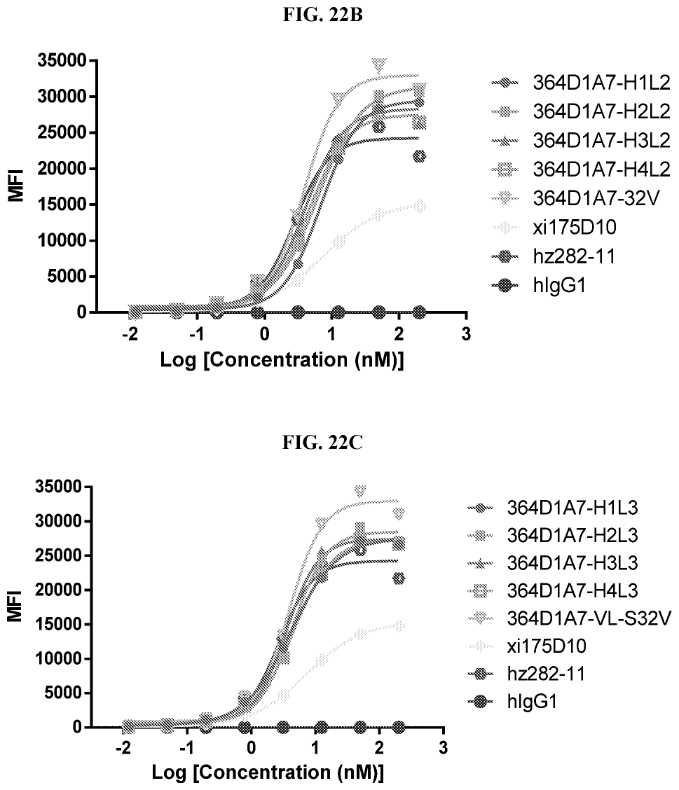

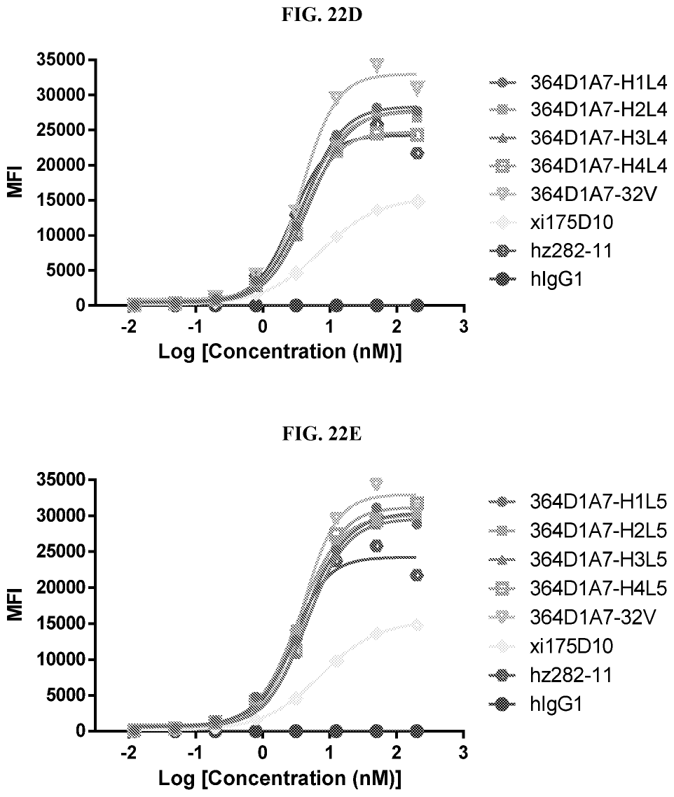

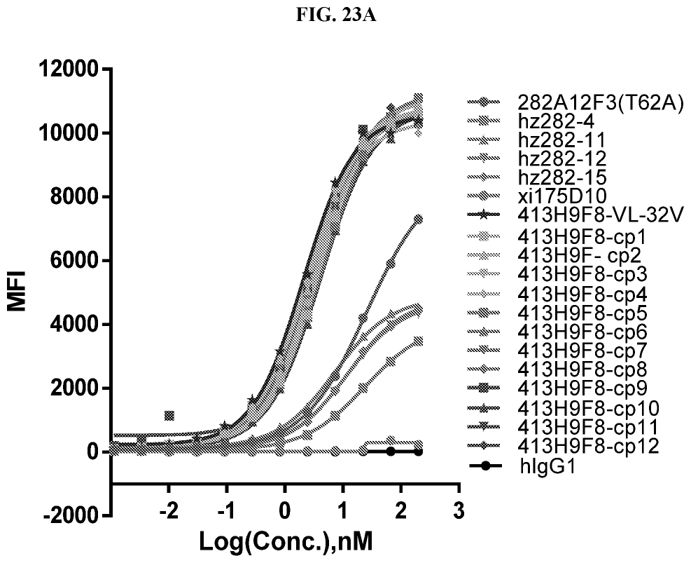

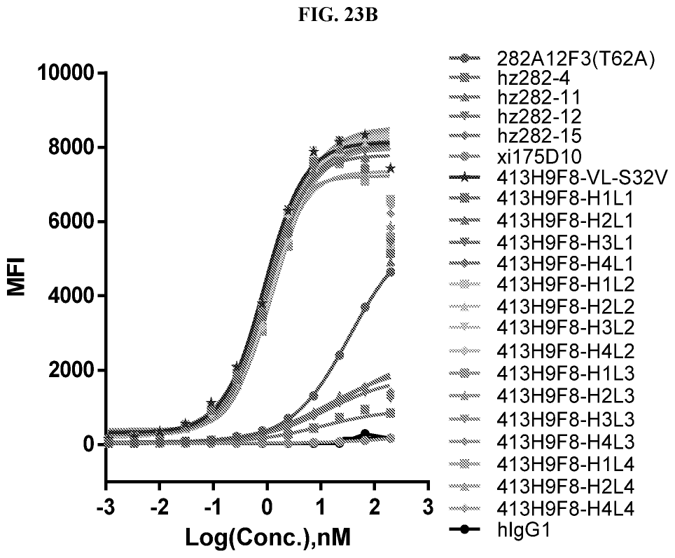

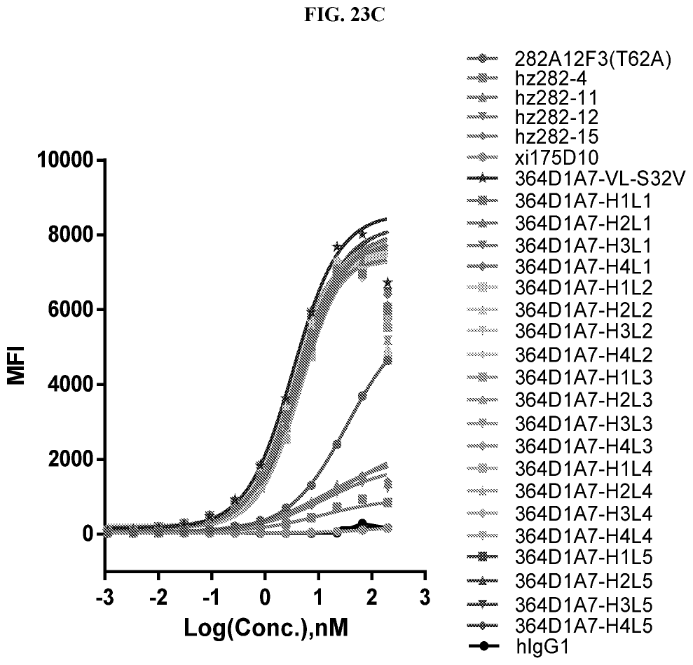

The binding affinities and specificity of humanized 413H9F8-VL-S32V and 364D1A7-VL-S32V antibodies were compared with those of parental antibodies by FACS analysis using CHO-CLDN18.1 and CHO-CLDN18.2 cells. As shown in FIG. 20A-FIG. 20D, FIG. 21A-FIG. 21D, Table 17, and Table 18, all of the tested humanized 413H9F8-VL-S32V antibodies showed comparable binding affinities to 413H9F8-VL-S32V on CHO-CLDN18.2 cells. As shown in FIG. 22A-FIG. 22E and Table 19, all of the tested humanized 364D1A7-VL-S32V antibodies showed comparable binding affinities to 364D1A7-VL-S32V on CHO-CLDN18.2 cells. The binding affinities of humanized 413H9F8-VL-S32V and 364D1A7-VL-S32V antibodies were further validated on SNU620 gastric cancer cell. As shown in FIG. 23A-FIG. 23C, all of the tested humanized 413H9F8-VL-S32V and 364D1A7-VL-S32V antibodies showed similar affinities to SNU620 gastric cancer cells compared with their parental antibodies. The data indicates that the humanized 413H9F8-VL-S32V and 364D1A7-VL-S32V antibodies retained binding specificity and affinity.

TABLE 17
Binding EC₅₀ₛ of humanized 413H9F8-VL-S32V antibodies
(strategy 1) on CHO-CLDN18.2 cells.

| Antibodies | EC₅₀(nM) |
|---|---|
| 413H9F8-cp1 | 2.60 |
| 413H9F8-cp2 | 1.99 |
| 413H9F8-cp3 | 2.09 |
| 413H9F8-cp4 | 2.28 |
| 413H9F8-cp5 | 2.54 |
| 413H9F8-cp6 | 2.32 |
| 413H9F8-cp7 | 2.47 |
| 413H9F8-cp8 | 2.72 |
| 413H9F8-cp9 | 2.21 |
| 413H9F8-cp10 | 3.69 |
| 413H9F8-cp11 | 2.61 |
| 413H9F8-cp12 | 3.01 |
| 413H9F8-VL-32V | 1.84 |
| xi175D10 | 9.32 |
| hz282-11 | 3.68 |
| 282A12F3(T62A) | 4.45 |

TABLE 18
Binding EC₅₀ₛ of humanized 413H9F8-VL-S32V
antibodies (in strategy 2) on CHO-CLDN18.2.

| Antibodies | EC₅₀ (nM) |
|---|---|
| 413H9F8-H1L1 | 2.31 |
| 413H9F8-H2L1 | 2.43 |
| 413H9F8-H3L1 | 2.42 |
| 413H9F8-H4L1 | 2.69 |
| 413H9F8-H1L2 | 2.18 |
| 413H9F8-H2L2 | 1.97 |
| 413H9F8-H3L2 | 2.14 |
| 413H9F8-H4L2 | 2.45 |
| 413H9F8-H1L3 | 2.38 |
| 413H9F8-H2L3 | 2.49 |
| 413H9F8-H3L3 | 2.24 |
| 413H9F8-H4L3 | 2.62 |
| 413H9F8-H1L4 | 3.32 |
| 413H9F8-H2L4 | 2.80 |
| 413H9F8-H3L4 | 2.74 |
| 413H9F8-H4L4 | 2.34 |
| 413H9F8-VL-32V | 2.14 |
| xi175D10 | 7.52 |
| hz282-11 | 3.17 |

TABLE 19
Binding EC₅₀ₛ of humanized 364D1A7
antibodies on CHO-CLDN18.2.

| Antibodies | EC₅₀ (nM) |
|---|---|
| 364D1A7-H1L1 | 3.50 |
| 364D1A7-H2L1 | 3.47 |
| 364D1A7-H3L1 | 4.22 |
| 364D1A7-H4L1 | 4.12 |
| 364D1A7-H1L2 | 6.90 |
| 364D1A7-H2L2 | 6.14 |
| 364D1A7-H3L2 | 4.05 |
| 364D1A7-H4L2 | 4.51 |
| 364D1A7-H1L3 | 4.10 |
| 364D1A7-H2L3 | 4.40 |
| 364D1A7-H3L3 | 3.54 |
| 364D1A7-H4L3 | 4.56 |
| 364D1A7-H1L4 | 4.03 |
| 364D1A7-H2L4 | 3.94 |
| 364D1A7-H3L4 | 4.78 |
| 364D1A7-H4L4 | 3.85 |
| 364D1A7-H1L5 | 3.96 |
| 364D1A7-H2L5 | 3.35 |
| 364D1A7-H3L5 | 4.53 |
| 364D1A7-H4L5 | 4.38 |
| 364D1A7-VL-32V | 3.87 |
| Xi175D10 | 7.24 |
| hz282-11 | 2.75 |

### Example 16—ADCC Function of Exemplary Humanized Antibodies

CLDN18.2 specific ADCC activities of humanized antibodies were validated on CHO-CLDN18.1 and CHO-CLDN18.2 cell lines as described above. As shown in FIG. 14A-FIG. 14B, humanized antibodies 413H9F8-H1L1 and 364D1A7-H1L1 induced ADCC mediated lysis of CHO-CLDN18.2 but not CHO-CLDN18.1. It indicated that humanized antibodies retained the target specificity of their parental antibodies.

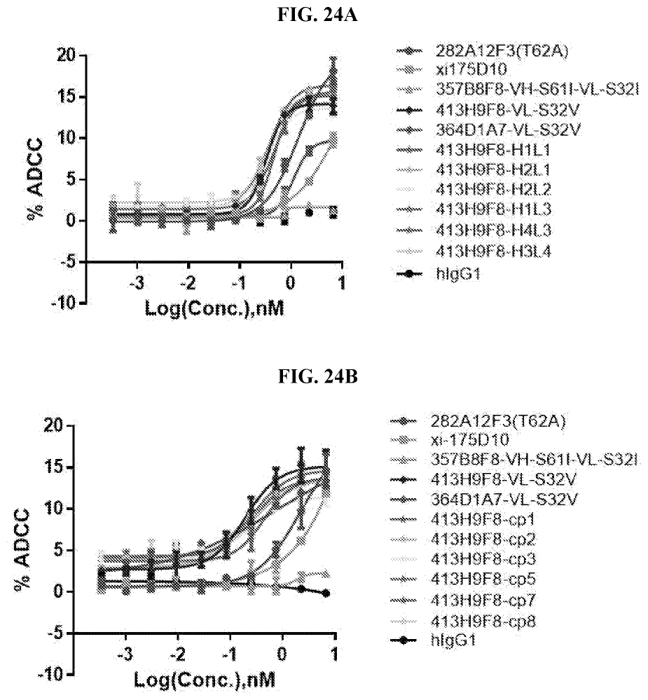

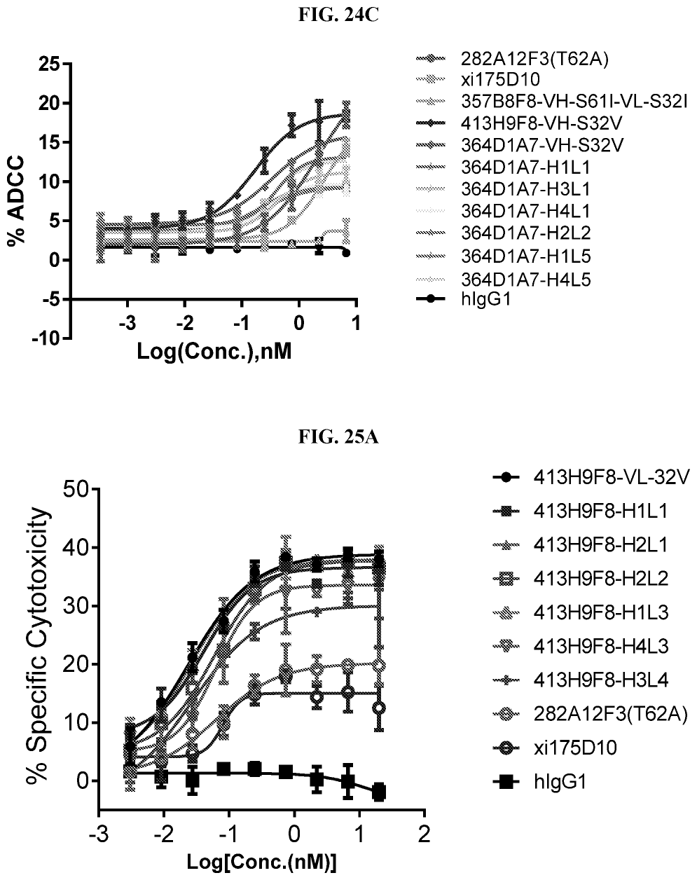

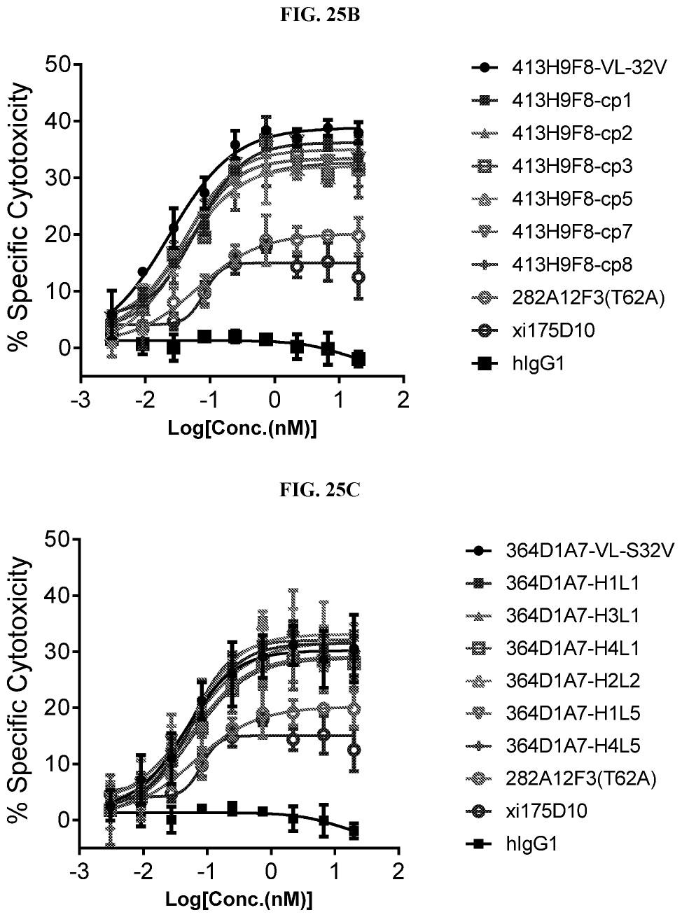

The ADCC efficacy of humanized antibody variants and parental antibodies were analyzed. Briefly, effector FcR-TANK (CD16A-15V) cells were mixed with CFSE labeled target cell NCI-N87-CLDN18.2 at an effector: target cell ratio of 8:1. Mixed cells were cultured with humanized antibody for 4 hours. ADCC efficacy was analyzed and calculated as described above. As shown in FIG. 24A-FIG. 24C and Table 20, almost all of the tested humanized 413H9F8-VL-S32V and 364D1A7-VL-S32V antibodies showed similar ADCC activities compared with their parental antibodies respectively. The ADCC activities of humanized antibodies were further tested with PBMCs against NUGC4-CLDN18.2 gastric cancer cells as described above. As shown in FIG. 25A-FIG. 25C and Table 21, almost all of the tested humanized 413H9F8-VL-S32V and 364D1A7-VL-S32V antibodies showed comparable ADCC activities as compared with their parental antibodies respectively.

TABLE 20
EC₅₀ₛ and maximal ADCC activities of humanized antibodies
of 413H9F8 and 364D1A7 antibodies with FcR-TANK (CD16A-
15V) cells against NCI-N87-CLDN18.2 gastric cancer cell.

| | | % ADCC |
|---|---|---|
| Antibodies | EC50, nM | @ 6.67 nM |
| 413H9F8-VL-32V | 0.33 | 13.9 |
| 413H9F8-H1L1 | 0.47 | 15.1 |
| 413H9F8-H2L1 | 0.31 | 14.1 |
| 413H9F8-H2L2 | 0.33 | 13.7 |
| 413H9F8-H1L3 | 0.37 | 16.3 |
| 413H9F8-H4L3 | 0.47 | 15.4 |
| 413H9F8-H3L4 | 0.35 | 16.0 |
| 413H9F8-cp1 | 0.44 | 13.3 |
| 413H9F8-cp2 | 0.24 | 13.2 |
| 413H9F8-cp3 | 0.22 | 11.1 |
| 413H9F8-cp5 | 0.27 | 14.1 |
| 413H9F8-cp7 | 0.28 | 13.3 |
| 413H9F8-cp8 | 0.27 | 13.1 |
| 364D1A7-VL-32V | 0.34 | 15.3 |
| 364D1A7-H1L1 | 0.40 | 13.5 |
| 364D1A7-H3L1 | 0.41 | 10.8 |
| 364D1A7-H4L1 | 0.31 | 9.3 |
| 364D1A7-H2L2 | 0.22 | 9.4 |
| 364D1A7-H1L5 | 0.26 | 9.1 |
| 364D1A7-H4L5 | 0.34 | 12.0 |
| Xi175D10 | na. | 11.9 |
| 282A12F3(T62A) | 2.25 | 14.6 |
| 357B8F8-VH-S61I-VL-S32I | 1.81 | 2.3 |
| hIgG1 | ~272 | −0.1 |

TABLE 21
The EC₅₀ₛ and maximal ADCC activities of humanized
antibodies of 413H9F8 and 364D1A7 antibodies with
PBMCs against NUGC4-CHO18.2 gastric cancer cell.

| | | % specific cytotoxicity |
|---|---|---|
| Antibody | EC₅₀, nM | @ 6.67 nM |
| 413H9F8-VL-S32V | 7.69 | 31.4 |
| 413H9F8-H1L1 | 8.98 | 27.0 |
| 413H9F8-H2L1 | 5.97 | 34.3 |
| 413H9F8-H2L2 | 6.14 | 33.2 |
| 413H9F8-H1L3 | 4.38 | 34.6 |
| 413H9F8-H4L3 | 7.01 | 27.8 |
| 413H9F8-H3L4 | 6.09 | 28.7 |
| 413H9F8-cp1 | 7.18 | 31.5 |
| 413H9F8-cp2 | 5.30 | 32.0 |
| 413H9F8-cp3 | 5.86 | 30.5 |
| 413H9F8-cp5 | 7.75 | 29.6 |
| 413H9F8-cp7 | 6.68 | 29.7 |
| 413H9F8-cp8 | 8.85 | 27.7 |
| 364D1A7-VL-32V | 6.36 | 26.6 |
| 364D1A7-H1L1 | 13.30 | 26.7 |
| 364D1A7-H3L1 | 11.27 | 26.2 |
| 364D1A7-H4L1 | 7.58 | 26.4 |
| 364D1A7-H2L2 | 9.69 | 26.6 |
| 364D1A7-H1L5 | 8.17 | 27.5 |
| 364D1A7-H4L5 | 6.40 | 26.4 |
| 357B8F8-VH-S61I-VL-S32I | 4.87 | 19.6 |
| 282A12F3(T62A) | 9.52* | 21.0 |
| Xi175D10 | 9.21 | 16.8 |
| hIgG1 | na. | −0.8 |

*Data from 1 donor. Other data are average of 3 donors

### Example 17 ADCC Activities of Fc Variants of Humanized Anti-CLDN18.2 Antibodies

There are 4 main allotypes of human IgG1, including G1m1 (D356/L358), G1m-1 (E356/M358), G1m3 (R214), and G1m17 (K214), differ in their heavy chain. The allotypes are inherited in a codominant Mendelian way, and various sets of combinations are found in African, White, and Mongoloid populations (PMID: 25368619, 26685205). Anti-CLDN18.2 antibodies that are included in the studies have Fc variants with D356/L358 or E356/M358. For example, the reference antibody Xi175D10 has a Fc variant with D356/L358. It is noticeable that antibodies with Fc variants of D356/L358 and E356/M358 have similar ADCC activities (data not shown). A “DL” name suffix was added to indicate antibodies with D356/L358 in the Fc, while for those of E356/M358 variant, no specific name suffix was added. Various Fc engineering approaches, including S239D/I332E and F243L/R292P/Y300LN305I/P396L mutations, have been developed to enhance effector functions of antibodies (PMID: 29070978).

Anti-CLDN18.2 antibodies with S239D/I332E or F243L/R292P/Y300LN305I/P396L Fc variants were generated to improve their effector functions. A “V2” name suffix was added for antibodies with S239D/I332E Fc variant, and a “MG” name suffix was added for antibodies with F243L/R292P/Y300LN305I/P396L Fc variant.

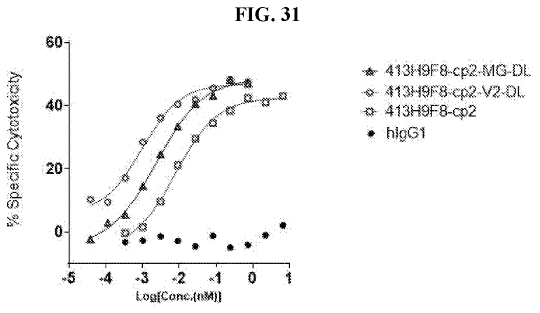

The ADCC effects of anti-CLDN18.2 antibodies with different Fc variants were evaluated on CHO-CLDN18.2 cell lines as described above. Briefly, effector FcR-TANK (CD16A-15V) cells were mixed with CFSE labeled target cell CHO-CLDN18.2 at an effector: target cell ratio of 4:1. Mixed cells were cultured with antibody for 4 hours. ADCC effect was analyzed and calculated as described above. As shown in FIG. 31 and Table 22, both 413H9F8-cp2-V2-DL and 413H9F8-cp2-MG-DL showed enhanced ADCC activities as compared with their parental antibodies 413H9F8-cp2.

TABLE 22
ADCC activities of 413H9F8-cp2 variants with FcR-
TANK (CD16A-15V) cells against CHO-CLDN18.2 cells.

| Antibodies | EC₅₀, nM |
|---|---|
| 413H9F8-cp2 | 0.0080 |
| 413H9F8-cp2-V2-DL | 0.0010 |
| 413H9F8-cp2-MG-DL | 0.0024 |
| hIgG1 | NA. |

NA. not applicable

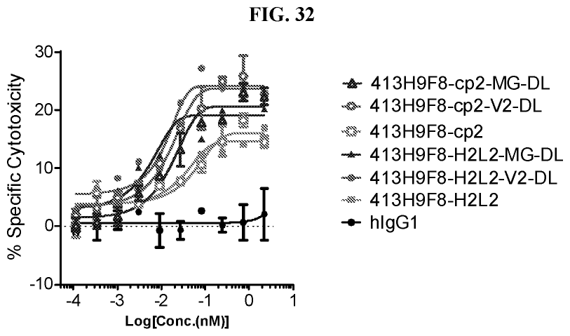

The ADCC activities of humanized antibodies were further tested with PBMCs against NUGC4-CLDN18.2 gastric cancer cells as described above. Humanized antibodies 413H9F8-cp2 and 413H9F8-H2L2 with different Fc variants were analyzed for their abilities to induce ADCC with human PBMCs against NUGC4-CLDN18.2 cells at an effector: target cell ratio of 40:1, cells were cultured for 5 hours. Data are generated from PBMCs derived from one healthy donor. Each data point represents average value of duplicates. As shown in FIG. 32 and Table 23, both “V2” and “MG” variants of 413H9F8-H2L2 and 413H9F8-cp2 showed enhanced ADCC activities as compared with their parental antibodies respectively.

TABLE 23
ADCC activities of 413H9F8-cp2 and 413H9F8-H2L2 variants with
human PBMCs against NUGC4-CLDN18.2 gastric cancer cell line.

| Antibodies | EC₅₀, nM |
|---|---|
| 413H9F8-cp2 | 0.0492 |
| 413H9F8-cp2-V2-DL | 0.0146 |
| 413H9F8-cp2-MG-DL | 0.0175 |
| 413H9F8-H2L2 | 0.480 |
| 413H9F8-H2L2-V2-DL | 0.0148 |
| 413H9F8-H2L2-MG-DL | 0.0046 |
| hIgG1 | NA. |

NA. not applicable

### Example 18—CDC Activities of Selected Humanized Antibodies

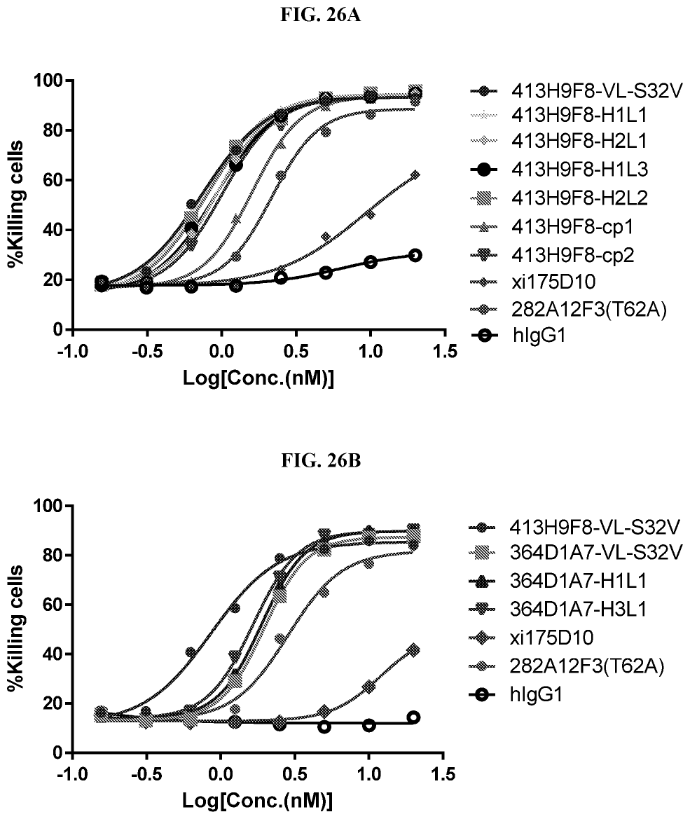

The CDC activities of humanized antibody variants were analyzed to compare their CDC function with parental antibodies as described above. As shown in FIG. 26A-FIG. 26B and Table 24, almost all of the tested humanized 413H9F8-VL-S32V and 364D1A7-VL-S32V clones showed similar CDC activities compared with their parental antibodies respectively.

TABLE 24
EC₅₀ₛ of selected humanized 413H9F8 and 364D1A7 clones
with human serum induced CDC against CHO-CHO18.2 cell.

| Antibody | CDC, EC₅₀, nM |
|---|---|
| 413H9F8-VL-S32V | 0.76 |
| 413H9F8-H1L1 | 0.78 |
| 413H9F8-H2L1 | 1.06 |
| 413H9F8-H2L2 | 0.84 |
| 413H9F8-H1L3 | 1.07 |
| 413H9F8-cp1 | 1.66 |
| 413H9F8-cp2 | 1.13 |
| 364D1A7-VL-S32V | 1.49 |
| 364D1A7-H1L1 | 1.48 |
| 364D1A7-H3L1 | 1.28 |
| xi175D10 | 2.26 |
| xi-282(T62A) | 10.43 |

### Example 19 Internalization of Anti-CLDN18.2 Antibodies by Tumor Cells

Internalization of antibody by tumor cells was determined indirectly by detecting cell surface retention of antibody after incubation at 37° C. to induce antibody internalization. Briefly, NUGC4-CLDN18.2 and NCI-N87-CLDN18.2 cells were collected and washed with wash buffer (PBS with 1% FBS), and adjusted to 1×105 cell/50 μL. Antibodies were diluted to 20 μg/mL, 50 μL of diluted antibodies were mixed with cell at a volume ratio of 1:1, followed by incubation on ice-bath for 30 min. Cells were washed twice with pre-cooling wash buffer and resuspended with 800 μL pre-cooling wash buffer. 100 μL cell suspension was added into 96-well plate at different time points and incubated at 37° C. 200 μL of pre-cooling wash buffer was added to stop the endocytosis temperature condition of all samples. Samples incubated on ice-bath was set as control that had no or minimal internalization. After wash once, 100 μL/well of 2nd antibody (AF647-goat anti-human IgG Fc γ, Jackson, #109-606-170, 1:800 dilution) was added and incubated on ice-bath for 30 min. Cells were washed twice with pre-cooling wash buffer and resuspended with 200 μL pre-cooling wash buffer, and analyzed by flow cytometry (BD FACS Celesta).

% internalization of antibodies=[MFI (incubated on ice-bath)−MFI (incubated at 37° C. for different time)]/MFI (incubated on ice-bath)×100%.

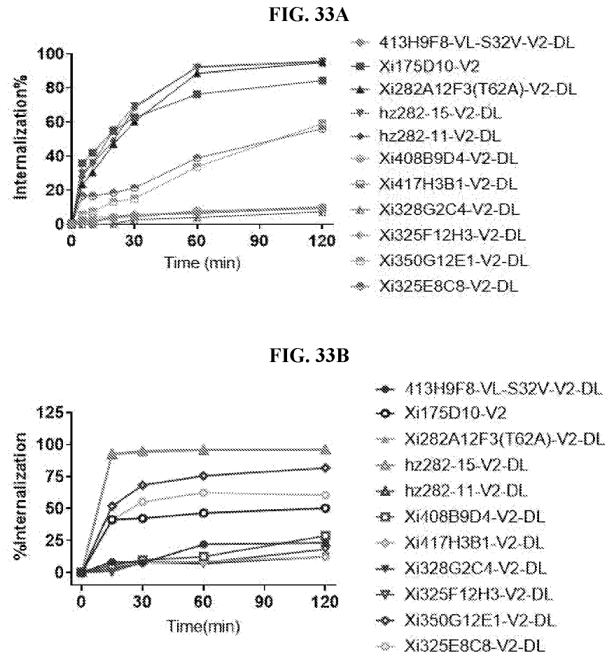

As shown in FIG. 33 A, Xi175D10-V2 and 282A12F3 (including Xi282A12F3(T62A)-V2-DL, hz282-11-V2 and hz282-15-V2 variants) were quickly internalized by NUGC4-CLDN18.2 cells and more than 80% of the antibodies were internalized after incubation at 37° C. for 2 hours. About 50% of Xi350G12E1-V2-DL and Xi325E8C80V2-DL were internalized by NUGC4-CLDN18.2 cells after incubation at 37° C. for 2 hours. It was noticeable that less than 15% of 413H9F8-VL-S32V-V2-DL and Xi408B9D4-V2-DL, Xi417H3B1-V2-DL, Xi328G2C4-V2-DL and Xi325F12H3-V2-DL were internalized by NUGC4-CLDN18.2 cells after incubation at 37° C. for 2 hours. NCI-N87-CLDN18.2 cells were also employed for internalization assay (FIG. 33 B). Specifically, more than 50% of Xi175D10-V2, 282A12F3 (including Xi282A12F3(T62A)-V2-DL, hz282-11-V2 and hz282-15-V2 variants), xi350G12E1-V2-DL and Xi325E8C80V2-DL were internalized by NCI-N87-CLDN18.2 cells after incubation at 37° C. for 2 hours. Less than 30% of 413H9F8-VL-S32V-V2-DL, Xi408B9D4-V2-DL, Xi417H3B1-V2-DL, Xi328G2C4-V2-DL and Xi325F12H3-V2-DL were internalized by NCI-N87-CLDN18.2 cells after incubation at 37° C. for 2 hours.

Collectively, engagement of 282A12F3 to CLDN18.2 overexpressed on gastric cancer cell lines leads to high level internalization of the antibody, whereas antibodies 413H9F8-VL-S32V-V2-DL, Xi408B9D4-V2-DL, Xi417H3B1-V2-DL, Xi328G2C4-V2-DL and Xi325F12H3-V2-DL only triggers minimal to mild antibody internalization, antibodies Xi175D10-V2, Xi325E8C80V2-DL and xi350G12E1-V2-DL induce medium to high level internalization.

### Example 20—Antibody-Drug Conjugation

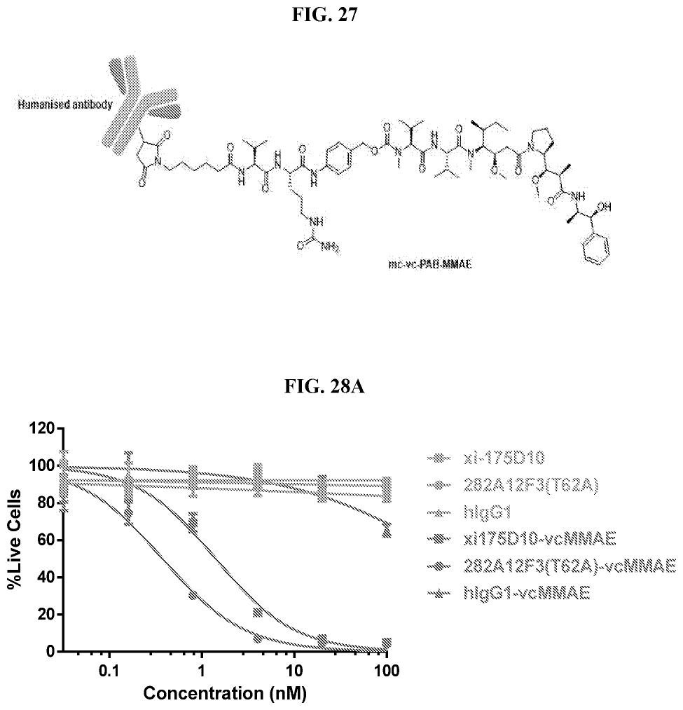

Naked antibodies 175D10 (xi175D10), 282A12F3(T62A) and isotype control antibody human IgG1 were conjugated to mc-vc-PAB-MMAE, a monomethyl auristatin E (MMAE) derivative comprising a cleavable valine-citrulline (vc) linker (FIG. 27). Briefly, antibodies were thawed in a 4° C. refrigerator for over 4 hours and dialyzed against conjugation buffer (25 mM Na₂B₄O₇, 25 mM NaCl, 1 mM DTPA, pH 7.4) at 4° C. overnight. Antibodies were reduced by adding freshly prepared TCEP working solution (5 mM TCEP in cysteine-maleimide conjugation buffer, TCEP-HCl), incubating in a 25° C. water-bath for 2 hours. Antibody was conjugated with freshly prepared mc-vc-PAB-MMAE (XDCExplorer) working solution in DMSO (10 mM) at a ratio of 6 in the presence of 10% v/v Organic Solvent (DMSO), incubating the mixture in a 25° C. water-bath for 2 hours. The antibody-drug conjugation was dialyzed against L-Histidine dialysis buffer (20 mM L-Histidine, pH 5.5) at 4° C. overnight, with one dialysis buffer exchange after 4 hours. Final product was extracted and filtered with 0.2m filter, and the quality was analyzed by HIC-HPLC. HIC-HPLC result showed that the drug-to-antibody ratio (DAR) value of all conjugates ranged from 3.5 to 4.0 (Table 25). With the increase in the TCEP molar ratio, the DAR value of ADC also increased.

TABLE 25
DAR value of ADCs.

| | TCEP | | | | | | Average |
|---|---|---|---|---|---|---|---|
| | molar | DAR₀ | DAR₂ | DAR₄ | DAR₆ | DAR₈ | DAR |
| ADC | ratio | (%) | (%) | (%) | (%) | (%) | (%) |
| Human IgG1 | 2.1 | 6.76 | 31.75 | 43.6 | 15.05 | 2.84 | 3.51 |
| control | 2.3 | 4.65 | 29.74 | 44.6 | 17.46 | 3.55 | 3.71 |
| | 2.5 | 3.7 | 26.43 | 45.34 | 19.52 | 5.01 | 3.91 |
| 282A12F3 | 2.1 | 4.61 | 31.93 | 45.89 | 15.72 | 1.85 | 3.57 |
| | 2.3 | 3.77 | 30.1 | 46.04 | 17.69 | 2.4 | 3.70 |
| | 2.5 | 2.79 | 25.22 | 47.96 | 20.33 | 3.69 | 3.94 |
| Xi175D10 | 2.1 | 2.28 | 28.98 | 50.77 | 16.22 | 1.74 | 3.72 |
| | 2.3 | 1.62 | 25.5 | 53.39 | 17.79 | 1.69 | 3.85 |
| | 2.5 | 1.28 | 21.34 | 55.29 | 19.4 | 2.69 | 4.02 |

### Example 21—Cell Killing Activities of ADCs on HEK293-CLDN18.2 Cells

Cytotoxicity of xi-175D10-vcMMAE, 282A12F3(T62A)-vcMMAE, and huIgG1-vcMMAE (with 3 different DARs) as well as corresponding naked antibodies was tested on HEK29 with over-expressing of CLDN18.2 (HK293-CLDN18.2) cell line. Briefly, HK293-CLDN18.2 cells were seeded in 96-well plate and grown overnight at 37° C., 5% CO₂. Naked antibodies and ADCs were prepared at 4× concentration (60 μg/mL, 400 nM) and were 5-fold serial diluted in cell growth medium. 100 μL of primary dilution was mixed with 100 μL medium to make 2× concentration. 50 μL of each complex dilution was added to the cells in triplicate. Cells were incubated for 5 days at 37° C., 5% CO₂. Cytotoxicity was determined by CellTiter Glo Luminescent Cell Viability Assay kit (Promega). 100 μl/well CellTiter Glo reagent was added for cell viability read-out. Incubated at room temperature on shaker for 10 minutes, recorded Luminescence on Envision.

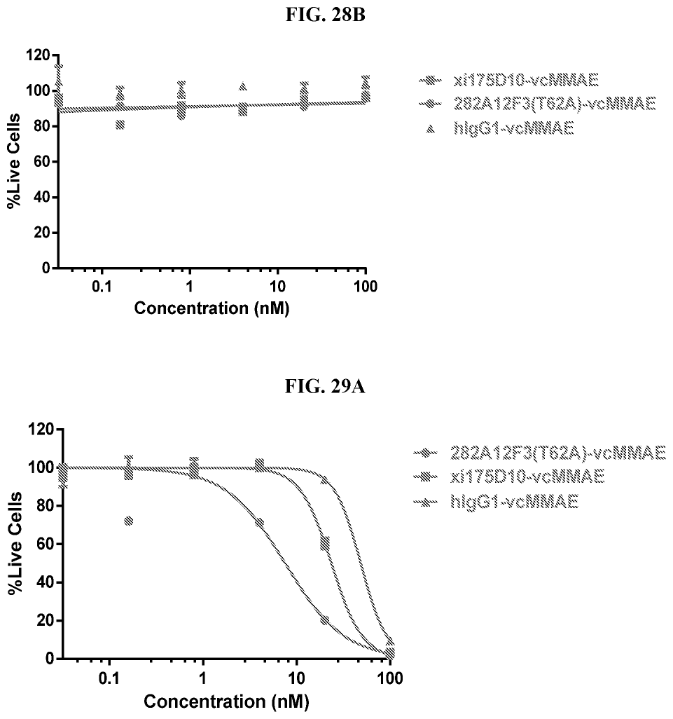

As shown in FIG. 28A-FIG. 28B, cell viability was not affected by treatment with naked antibodies, whereas, cell viability was decreased in a concentration dependent manner as treated with ADCs 282A12F3 (T62A)-vcMMAE and xi175D10-vcMMAE. Moreover, 282A12F3 (T62A)-vcMMAE was more efficient in inducing cell death compared with xi175D10-vcMMAE. ADCs 282A12F3 (T62A)-vcMMAE and xi175D10-vcMMAE did not affect the viability of HEK293 cell, which is CLDN18.2 negative. It indicates that ADCs 282A12F3 (T62A)-vcMMAE and xi175D10-vcMMAE specifically inhibit the viability of CLDN18.2 positive cells.

### Example 22—Cell Killing Activities of ADCs on NCI-N87-CLDN18.2 Cells

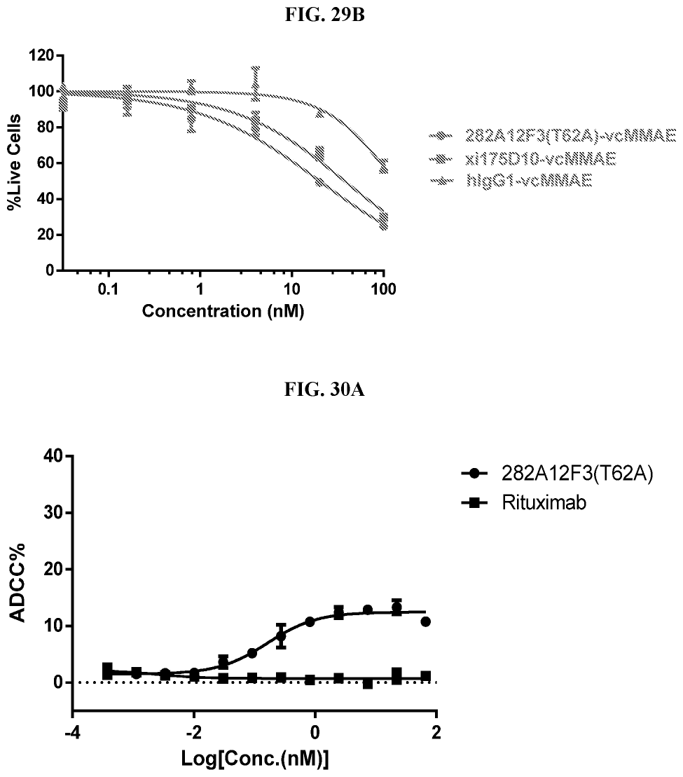

Two gastric cancer cell lines with over-expressing CLDN18.2, NCI-N87-CLDN18.2 and NUGC4-CLDN18.2 cells, were used to test the cell killing activities of ADCs as described above. As shown in FIG. 29A-FIG. 29B, cell viability was decreased in a concentration dependent manner as treated with ADCs 282A12F3 (T62A)-vcMMAE and xi175D10-vcMMAE. Moreover, 282A12 (T62A)-vcMMAE ADCs induced higher cell kill than xi175D10-vcMMAE. NUGC4-CLDN18.2 was less sensitive to ADCs induced cell death as compared to NCI-N87-CLDN18.2 cell.

### Example 23—Cell Killing Activities of ADCs on Cells that are Less Sensitive to ADCC

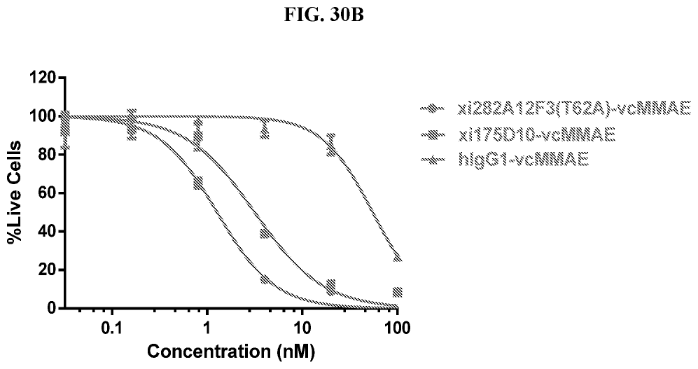

Pancreatic cancer cell line PANC-1-CLDN18.2 which was stably transfected with CLDN18.2 and had been shown to be less sensitive to chimeric 282A12F3 (T62A) mediated ADCC efficacy (FIG. 30A) were used in the ADC-dependent cell killing assay. As shown in FIG. 30B, both 282A12F3 (T62A)-vcMMAE and xi175D10-vcMMAE inhibited the viability of PANC-1-CLDN18.2 cell in concentration dependent manners, and 282A12F3 (T62A)-vcMMAE was more potent than xi175D10-vcMMAE in inducing cell death of PANC-1-CLDN18.2 cells.

In summary, anti-CLDN18.2-ADCs killed cell lines that overexpressing CLDN18.2, including HEK293-CLDN18.2, NCI-N87-CLDN18.2, and NUGC4-CLDN18.2. 282A12F3 (T62A)-vcMMAE was more potent than xi175D10-vcMMAE in inhibiting viability of tested cell lines. Moreover, drug-antibody-ratio in the range of between 3.5 and 4.0 was not observed to modulate cell killing activities of the ADCs (Table 26).

TABLE 26
Cell killing activities of CLDN18.2 specific ADCs

| | CLDN18.2 positive cell lines | | | | | | | | | CLDN18.2 | |
|---|---|---|---|---|---|---|---|---|---|---|---|
| | HEK293- | | | Panc1- | | NCI-N87- | | NUGC4- | | negative cell line | |
| | CLDN18.2 | | | CLDN18.2 | | CLDN18.2 | | CLDN18.2 | | HEK293 | |
| | | | Killing | | Killing | | Killing | | Killing | | Killing |
| | | IC₅₀ | (%) @ | IC₅₀ | (%) @ | IC₅₀ | (%) @ | IC₅₀ | (%) @ | IC₅₀ | (%) @ |
| ADC | DAR | (nM) | 20 nM | (nM) | 20 nM | (nM) | 20 nM | (nM) | 20 nM | (nM) | 20 nM |
| 282A12F3 | 3.57 | 0.44 | 95.0 | 1.5 | 90.8 | 10.8 | 74.4 | 25.6 | 45.8 | na | 1.3 |
| (T62A)-vcMMAE | 3.7 | 0.35 | 95.4 | 1.1 | 92.4 | 9.0 | 78.7 | 21.7 | 45.3 | na | 0.8 |
| | 3.94 | 0.38 | 95.2 | 1.3 | 91.2 | 7.6 | 79.9 | 20.1 | 50.7 | na | 8.9 |
| xi175D10- | 3.72 | 1.51 | 93.7 | 3.3 | 86.5 | 23.0 | 42.6 | 38.3 | 35.5 | na | 9.8 |
| vcMMAE | 3.85 | 1.23 | 93.3 | 3.0 | 87.4 | 21.4 | 46.2 | 39.0 | 34.8 | na | 10.5 |
| | 4.02 | 1.41 | 93.0 | 3.1 | 87.5 | 23.6 | 39.6 | 37.6 | 34.7 | na | 4.8 |
| hIgG1-vcMMAE | 3.51 | na | 17.2 | 58.7 | 14.5 | 53.6 | 4.1 | 181.2 | 9.9 | na | 1.8 |
| | 3.71 | na | 13.1 | 49.2 | 21.7 | 49.7 | 6.7 | 222.0 | 14.3 | na | 2.4 |
| | 3.91 | na | 8.5 | 55.0 | 15.0 | 48.5 | 6.1 | 130.8 | 12.1 | na | −1.7 |

### Example 24 Efficacies of Anti-CLDN18.2 Antibodies in Human Gastric Cancer GA0006 Patient Derived Xenograft (PDX) Model in Nude Mice

The in vivo efficacies of anti-CLDN18.2 antibodies were tested in patient derived xenograft (PDX) model in nude mice. GA0006 was derived from the stomach of an Asian gastric cancer patient with the pathological diagnosis of adenocarcinoma type, multicopy of ERBB2. High expression of CLDN18.2 on GA0006 was confirmed by IHC and FACS analyses with anti-CLDN18.2 antibodies (data not shown). BALB/c nude mice were subcutaneously inoculated with tumors of about 3 mm×3 mm×3 mm in size into the right ankle. Mice were randomly divided into 8 groups (8 mice per group): PBS, hIgG1 isotype (100 mg/kg), xi175D10-V2, 413H9F8-H2L2-V2-DL and 413H9F8-cp2-V2-DL (50 and 100 mg/kg), when the average tumor size reached about 100 mm³. The coefficient of variation for tumor-volume was less than 40%, which was calculated by formula: CV=SD/MTV×100%. The day of randomization was recorded as day 0. Treatment of mice was initiated at day 0. Antibodies were administered 3 times per week for 3 weeks with alternating intravenous and intraperitoneal injection. Tumor sizes were monitored twice a week.

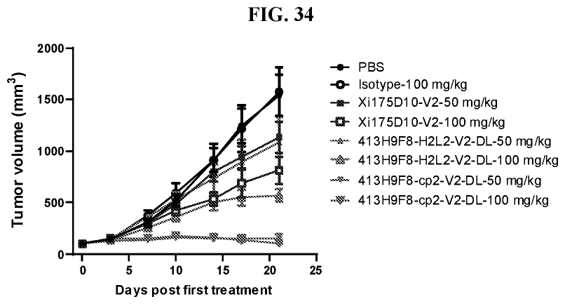

As shown in FIG. 34, 50 mg/kg of xi175D10-V2 or 413H9F8-H2L2-V2-DL treatment retarded tumor growth as compared with isotype (100 mg/kg) group, though did not achieve significant difference (p>0.05). 100 mg/kg of xi175D10-V2 and 413H9F8-H2L2-V2-DL treatment significantly inhibited tumor growth as compared with those treated with isotype (100 mg/kg) (p<0.05 and p<0.01). Both 50 and 100 mg/kg of 413H9F8-cp2-V2-DL treatment significantly inhibited tumor growth as compared with those treated with isotype (100 mg/kg) (p<0.0001). 50 mg/kg of 413H9F8-cp2-V2-DL treatment significantly inhibited tumor growth as compared with those treated with xi175D10-V2 (50 mg/kg) and 413H9F8-H2L2-V2-DL (50 mg/kg) (p<0.0001 and p<0.01). 100 mg/kg of 413H9F8-cp2-V2-DL treatment significantly inhibited tumor growth as compared with those treated with xi175D10-V2 (100 mg/kg) and 413H9F8-H2L2-V2-DL (100 mg/kg) (p<0.001 and p<0.0001).

### Example 25 Efficacies of Anti-CLDN18.2 Antibodies in Mouse Xenograft Models of Pancreatic Cancer in Nu/Nu Mice

The in vivo efficacies of anti-CLDN18.2 antibodies were tested in subcutaneous xenograft models of pancreatic cancer in Nu/Nu mice. Pancreatic cancer cell line MIA Paca-2 overexpressing CLDN18.2 (MIA Paca-2-CLDN18.2) was maintained in vitro as a monolayer culture in DMEM medium supplemented with 10% fetal bovine serum, 2.5% horse serum, 1% penicillin/streptomycin, 5 μg/mL of blasticidin, at 37° C. in an atmosphere of 5% CO₂ in air. MIA Paca-2-CLDN18.2 cells were routinely sub-cultured twice weekly by trypsin-EDTA treatment. MIA Paca-2-CLDN18.2 cells growing in an exponential growth phase were harvested and counted for tumor inoculation. Nu/Nu nude mice, female, 4-6 weeks, were inoculated subcutaneously at the right flank with 5×10⁶ MIA Paca-2-CLDN18.2 cells in 0.2 ml of PBS (supplemented with Matrigel, PBS:Matrigel=1:1) for tumor development. Treatment of Paca-2-CLDN18.2 tumor bearing Nu/Nu mice (10 mice per group) were initiated 3 days after tumor inoculation. Anti-CLDN18.2 antibodies (10 and 40 mg/kg) were administered 2 times per week for 5 weeks with alternating intravenous and intraperitoneal injection. Tumor bearing Nu/Nu mice treated with PBS or isotype (hIgG1, 40 mg/kg) were set as negative control.

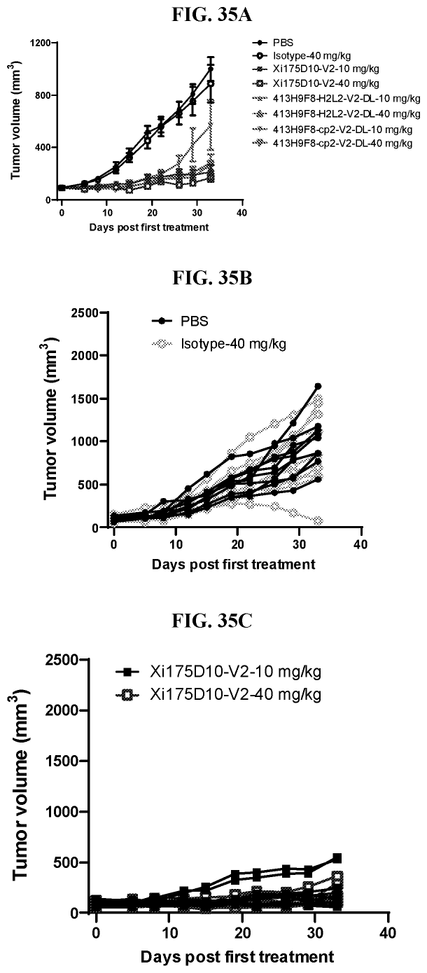

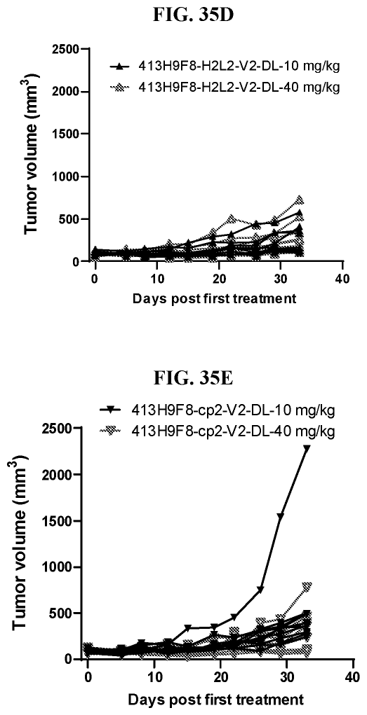

Mice treated with Xi175D10-V2, 413H9F8-H2L2-V2-DL at 10 and 40 mg/kg showed significant tumor growth retardation as compared with mice treated with PBS or isotype (40 mg/kg) (p<0.01) (FIG. 35 A-D). Mice treated with 413H9F8-cp2-V2-DL at 40 mg/kg significantly inhibited tumor growth as compared with those treated with PBS or isotype (40 mg/kg) (p<0.01) (FIGS. 35A and E). Mice treated with 413H9F8-cp2-V2-DL at 10 mg/kg inhibited tumor growth, but not of significant difference as compared with those treated with PBS or isotype (40 mg/kg) (FIGS. 35 A and E).

### Example 26 Combinatorial Efficacies of Anti-CLD1N8.2 Antibodies and Chemotherapy in Human Gastric Cancer GA0006 Patient Derived Xenograft (PDX) Model

PDX mice model were established as described above. Treatment of mice was initiated at day 0. Tumor bearing mice were treated with PBS, EOF (1.25 mg/kg epirubicin, 3.25 mg/kg oxaliplatin and 56.25 mg/kg 5-fluorouracil), xi175D10-V2 (40 mg/kg) combined with EOF or 413H9F8-H2L2-V2-DL (40 mg/kg) combined with EOF. EOF were administered intraperitoneally once a week. Antibodies were administered 3 times per week by alternating intravenous and intraperitoneal injection. Tumor size was monitored twice a week. In total, 5 times of EOF administration and 14 times of antibodies treatment were conducted.

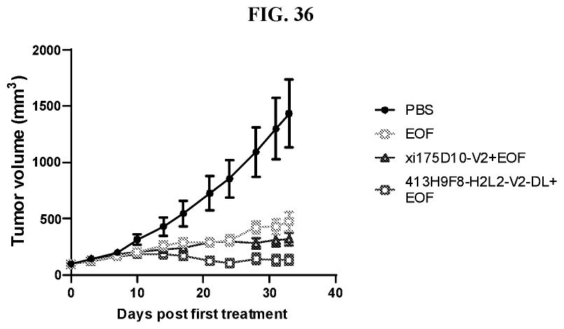

As shown in FIG. 36, treatment with EOF alone or EOF combined with xi175D10-V2 or 413H9F8-H2L2-V2-DL significantly inhibited tumor growth as compared with those treated with PBS (p<0.01). 413H9F8-H2L2-V2-DL combined with EOF showed superior effects than EOF therapy alone (p<0.01). However, xi175D10-V2 combined with EOF did not show better effects as compared with EOF therapy alone (p=0.147). Moreover, combo of 413H9F8-H2L2-V2-DL with EOF was superior than combo of xi175D10-V2 with EOF (p<0.05).

### Example 27

Table 27
illustrates the heavy chain and
light chain sequences of reference
antibody 175D10 (xi175D10).

| | | SEQ |
|---|---|---|
| | | ID |
| | SEQUENCE | NO: |
| 175D10 | MGWSCIILFLVATATGVHSQVQLQQPG | 98 |
| Heavy | AELVRPGASVKLSCKASGYTFTSYWIN | |
| Chain | WVKQRPGQGLEWIGNIYPSDSYTNYNQ | |
| | KFKDKATLTVDKSSSTAYMQLSSPTSE | |
| | DSAVYYCTRSWRGNSFDYWGQGTTLTV | |
| | SSASTKGPSVFPLAPSSKSTSGGTAAL | |
| | GCLVKDYFPEPVTVSWNSGALTSGVHT | |
| | FPAVLQSSGLYSLSSVVTVPSSSLGTQ | |
| | TYICNVNHKPSNTKVDKKVEPKSCDKT | |
| | HTCPPCPAPELLGGPSVFLFPPKPKDT | |
| | LMISRTPEVTCVVVDVSHEDPEVKFNW | |
| | YVDGVEVHNAKTKPREEQYNSTYRVVS | |
| | VLTVLHQDWLNGKEYKCKVSNKALPAP | |
| | IEKTISKAKGQPREPQVYTLPPSRDEL | |
| | TKNQVSLTCLVKGFYPSDIAVEWESNG | |
| | QPENNYKTTPPVLDSDGSFFLYSKLTV | |
| | DKSRWQQGNVFSCSVMHEALHNHYTQK | |
| | SLSLSPGK | |
| 175D10 | MESQTQVLMSLLFWVSGTCGDIVMTQS | 99 |
| Light | PSSLTVTAGEKVTMSCKSSQSLLNSGN | |
| Chain | QKNYLTWYQQKPGQPPKLLIYWASTRE | |
| | SGVPDRFTGSGSGTDFTLTISSVQAED | |
| | LAVYYCQNDYSYPFTFGSGTKLEIKRT | |
| | VAAPSVFIFPPSDEQLKSGTASVVCLL | |
| | NNFYPREAKVQWKVDNALQSGNSQESV | |
| | TEQDSKDSTYSLSSTLTLSKADYEKHK | |
| | VYACEVTHQGLSSPVTKSFNRGEC | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. An anti-Claudin 18.2 (anti-CLDN18.2) antibody comprising a half maximal effective concentration (EC50) that is lower than an EC50 of reference antibody 175D10, wherein the reference antibody 175D10 comprises a heavy chain (HC) sequence set forth in SEQ ID NO: 98 and a light chain (LC) sequence set forth in SEQ ID NO: 99.

2. An anti-Claudin 18.2 (anti-CLDN18.2) antibody comprising at least one mutation at a post-translational modification site.

3. An anti-Claudin 18.2 (anti-CLDN18.2) antibody comprising at least one mutation at a Fc region that confer enhanced antibody-dependent cell-mediated cytotoxicity (ADCC), wherein the enhanced ADCC is compared to reference antibody 175D10 comprising a heavy chain (HC) sequence set forth in SEQ ID NO: 98 and a light chain (LC) sequence set forth in SEQ ID NO: 99.

4. The anti-CLDN18.2 antibody of claim 1, wherein the EC50 of the anti-CLDN18.2 antibody is about 5 nM or lower.

5. The anti-CLDN18.2 antibody of claim 1, wherein the EC50 of the anti-CLDN18.2 antibody is about 5 nM, about 4 nM, about 3 nM, about 2 nM, about 1 nM, about 0.5 nM, or lower.

6. An anti-Claudin 18.2 (anti-CLDN18.2) antibody comprising a higher binding affinity to CLDN18.2 relative to a binding affinity of reference antibody 175D10, wherein the reference antibody 175D10 comprises a heavy chain (HC) sequence set forth in SEQ ID NO: 98 and a light chain (LC) sequence set forth in SEQ ID NO: 99.

7. The anti-CLDN18.2 antibody of any one of the claims 1-6, wherein the anti-CLDN18.2 antibody comprises a variable heavy chain (VH) region and a variable light chain (VL) region, wherein the VH region comprises:
CDR1 sequence GFSLTSYX₁VX₂;
wherein X₁ is selected from N or G; and X₂ is selected from Y or H;
CDR2 sequence VIWX₃X₄GX₅TX₆YX₇X₈X₉LX₁₀S;
wherein X₃ is selected from N or P; X₄ is selected from T or G; X₅ is selected from A or N; X₆ is selected from R or N; X₇ is selected from N, Q, or E; X₈ is selected from S or I; X₉ is selected from T or A; and X₁₀ is selected from K or M; and
CDR3 sequence DX₁₁X₁₂X₁₃X₁₄X₁₅X₁₆X₁₇X₁₈X₁₉X₂₀;
wherein X₁₁ is selected from S or R; X₁₂ is selected from A or R; X₁₃ is selected from M or L; X₁₄ is selected from P or A; X₁₅ is selected from A or M; X₁₆ is selected from I or D; X₁₇ is selected from P or Y; X₁₈ is present or absence, if present, is F; X₁₉ is present or absence, if present, is A; and X₂₀ is present or absence, if present, is Y.

8. The anti-CLDN18.2 antibody of any one of the claims 1-6, wherein the VH region comprises
CDR1 sequence X₂₁X₂₂X₂₃X₂₄X₂₅ SFGMH;
wherein X₂₁ is present or absence, if present, is G; X₂₂ is present or absence, if present, is F; X₂₃ is present or absence, if present, is T; X₂₄ is present or absence, if present, is F; and X₂₅ is present or absence, if present, is S;
CDR2 sequence YISSGSX₂₆X₂₇IYYX₂₈DX₂₉X₃₀KG;
wherein X₂₆ is selected from S or G; X₂₇ is selected from P or S; X₂₈ is selected from V or A; X₂₉ is selected from K or T; and X₃₀ is selected from L or V; and CDR3 sequence AX₃₁X₃₂X₃₃X₃₄X₃₅X₃₆X₃₇X₃₈X₃₉X₄₀X₄₁;
wherein X₃₁ is selected from G or T; X₃₂ is selected from Y or S; X₃₃ is selected from A or Y; X₃₄ is selected from V or Y; X₃₅ is selected from R or Y; X₃₆ is selected from N or G; X₃₇ is selected from A or N; X₃₈ is selected from L or A; X₃₉ is selected from D or L; X₄₀ is selected from Y or E; and X₄₁ is present or absence, if present, is Y.

9. The anti-CLDN18.2 antibody of any one of the claims 1-8, wherein the VH region comprises CDR1 sequence consisting of SEQ ID NO: 1, CDR2 sequence VIWNTGATRYX₇SX₉LKS, and CDR3 sequence consisting of SEQ ID NO: 3, wherein X₇ is selected from N, Q, or E; and X₉ is selected from T or A.

10. The anti-CLDN18.2 antibody of any one of the claims 1-8, wherein the VH region comprises CDR1 sequence consisting of SEQ ID NO: 13, CDR2 sequence VIWPGGNTNYX₇X₈ALMS, and CDR3 sequence consisting of SEQ ID NO: 15, wherein X₇ is selected from N or E; and X₈ is selected from S or I.

11. The anti-CLDN18.2 antibody of any one of the claims 1-10, wherein the VH region comprises CDR1 sequence selected from SEQ ID NOs: 1, 7, 10, or 13; CDR2 sequence selected from SEQ ID NOs: 2, 4, 5, 6, 8, 11, 14, 16, or 17; and CDR3 sequence selected from SEQ ID NOs: 3, 9, 12, or 15.

12. The anti-CLDN18.2 antibody of any one of the claims 1-10, wherein the VH region comprises CDR1 sequence consisting of SEQ ID NO: 1; CDR2 sequence selected from SEQ ID NOs: 2, 4, 5, or 6; and CDR3 sequence consisting of SEQ ID NO: 3.

13. The anti-CLDN18.2 antibody of any one of the claims 1-10, wherein the VH region comprises CDR1 sequence consisting of SEQ ID NO: 13; CDR2 sequence selected from SEQ ID NOs: 14, 16, or 17; and CDR3 sequence consisting of SEQ ID NO: 15.

14. The anti-CLDN18.2 antibody of any one of the claims 1-10, wherein the VH region comprises CDR1 sequence consisting of SEQ ID NO: 7, CDR2 sequence consisting of SEQ ID NO: 8, and CDR3 sequence consisting of SEQ ID NO: 9.

15. The anti-CLDN18.2 antibody of any one of the claims 1-10, wherein the VH region comprises CDR1 sequence consisting of SEQ ID NO: 10, CDR2 sequence consisting of SEQ ID NO: 11, and CDR3 sequence consisting of SEQ ID NO: 12.

16. The anti-CLDN18.2 antibody of any one of the claims 1-15, wherein the VL region comprises CDR1 sequence selected from SEQ ID NOs: 18, 21, 24-28, 31-35, 38, or 39; CDR2 sequence selected from SEQ ID NOs: 19, 22, 29, or 36; and CDR3 sequence selected from SEQ ID NOs: 20, 23, 30, or 37.

17. The anti-CLDN18.2 antibody of any one of the claims 1-15, wherein the VL region comprises CDR1 sequence selected from SEQ ID NOs: 21 or 24-27; CDR2 sequence consisting of SEQ ID NO: 22; and CDR3 sequence consisting of SEQ ID NO: 23.

18. The anti-CLDN18.2 antibody of any one of the claims 1-15, wherein the VL region comprises CDR1 sequence selected from SEQ ID NOs: 28 or 31-34; CDR2 sequence consisting of SEQ ID NO: 29; and CDR3 sequence consisting of SEQ ID NO: 30.

19. The anti-CLDN18.2 antibody of any one of the claims 1-15, wherein the VL region comprises CDR1 sequence selected from SEQ ID NOs: 35, 38, or 39; CDR2 sequence consisting of SEQ ID NO: 36; and CDR3 sequence consisting of SEQ ID NO: 37.

20. The anti-CLDN18.2 antibody of any one of the claims 1-15, wherein the VL region comprises CDR1 sequence consisting of SEQ ID NO: 18, CDR2 sequence consisting of SEQ ID NO: 19, and CDR3 sequence consisting of SEQ ID NO: 20.

21. The anti-CLDN18.2 antibody of any one of the claims 1-20, wherein the anti-CLDN18.2 antibody is a full-length antibody.

22. The anti-CLDN18.2 antibody of any one of the claims 1-20, wherein the anti-CLDN18.2 antibody is a binding fragment.

23. The anti-CLDN18.2 antibody of any one of the claims 1-22, wherein the anti-CLDN18.2 antibody comprises a monovalent Fab′, a divalent Fab2, a single-chain variable fragment (scFv), a diabody, a minibody, a nanobody, a single-domain antibody (sdAb), or a camelid antibody or binding fragment thereof.

24. The anti-CLDN18.2 antibody of any one of the claims 1-22, wherein the anti-CLDN18.2 antibody comprises a humanized antibody or binding fragment thereof, a chimeric antibody or binding fragment thereof, a monoclonal antibody or binding fragment thereof, or a bispecific antibody or binding fragment thereof.

25. The anti-CLDN18.2 antibody of any one of the claims 1-24, wherein the anti-CLDN18.2 antibody comprises a mutation at a post-translational modification site.

26. The anti-CLDN18.2 antibody of claim 25, wherein the mutation is at an amino acid position 60, 61, or 62 of a VH region, and wherein the amino acid positions correspond to position 60, 61, or 62 of SEQ ID NO: 40.

27. The anti-CLDN18.2 antibody of claim 26, wherein the mutation is at an amino acid position 60 or 62 of SEQ ID NO: 40.

28. The anti-CLDN18.2 antibody of claim 26, wherein the mutation is at an amino acid position 60 or 61 of SEQ ID NO: 57.

29. The anti-CLDN18.2 antibody of any one of the claims 26-28, wherein the mutation at amino acid residue N60 is to glutamine or glutamic acid.

30. The anti-CLDN18.2 antibody of any one of the claims 26-28, wherein the mutation at amino acid residue S61 is to isoleucine.

31. The anti-CLDN18.2 antibody of any one of the claims 26-28, wherein the mutation at amino acid residue T62 is to alanine.

32. The anti-CLDN18.2 antibody of claim 25, wherein the mutation is at an amino acid position 31 or 32 of a VL region, and wherein the amino acid positions correspond to position 31 or 32 of SEQ ID NO: 46, 52, or 60.

33. The anti-CLDN18.2 antibody of claim 32, wherein the mutation is at amino acid position 31 or 32 of SEQ ID NO: 46, 52, or 60.

34. The anti-CLDN18.2 antibody of claim 32 or 33, wherein the mutation at amino acid residue N31 is to aspartic acid or glutamic acid.

35. The anti-CLDN18.2 antibody of claim 32 or 33, wherein the mutation at amino acid residue S32 is to leucine, valine, or isoleucine.

36. The anti-CLDN18.2 antibody of any one of the claims 25-35, wherein the mutation enhances the binding affinity of the modified anti-CLDN18.2 antibody relative to the reference antibody 175D10.

37. The anti-CLDN18.2 antibody of any one of the claims 1-36, wherein the anti-CLDN18.2 antibody comprises a chimeric antibody or binding fragment thereof.

38. The anti-CLDN18.2 antibody of claim 37, wherein the chimeric antibody or binding fragment thereof comprises a VH region comprising at least 80%, 85%, 90%, 95%, or 100% sequence identity to SEQ ID NOs: 40-43 and a VL region comprising at least 80%, 85%, 90%, 95%, or 100% sequence identity to SEQ ID NO: 44.

39. The anti-CLDN18.2 antibody of claim 37, wherein the chimeric antibody or binding fragment thereof comprises a VH region comprising at least 80%, 85%, 90%, 95%, or 100% sequence identity to SEQ ID NO: 45 and a VL region comprising at least 80%, 85%, 90%, 95%, or 100% sequence identity to ID NOs: 46-50.

40. The anti-CLDN18.2 antibody of claim 37, wherein the chimeric antibody or binding fragment thereof comprises a VH region comprising at least 80%, 85%, 90%, 95%, or 100% sequence identity to SEQ ID NO: 51 and a VL region comprising at least 80%, 85%, 90%, 95%, or 100% sequence identity to SEQ ID NOs: 52-56.

41. The anti-CLDN18.2 antibody of claim 37, wherein the chimeric antibody or binding fragment thereof comprises a VH region comprising at least 80%, 85%, 90%, 95%, or 100% sequence identity to SEQ ID NOs: 57-59 and a VL region comprising at least 80%, 85%, 90%, 95%, or 100% sequence identity to SEQ ID NOs: 60-62.

42. The anti-CLDN18.2 antibody of any one of the claims 37-41, wherein the chimeric antibody or binding fragment thereof comprises a CH region comprising at least 80%, 85%, 90%, 95%, or 100% sequence identity to SEQ ID NO: 63 and a CL region comprising at least 80%, 85%, 90%, 95%, or 100% sequence identity to SEQ ID NO: 64.

43. The anti-CLDN18.2 antibody of any one of the claims 1-36, wherein the anti-CLDN18.2 antibody comprises a humanized antibody or binding fragment thereof.

44. The anti-CLDN18.2 antibody of claim 43, wherein the humanized antibody or binding fragment thereof comprises a VH region comprising at least 80%, 85%, 90%, 95%, or 100% sequence identity to SEQ ID NOs: 65-68 and a VL region comprising at least 80%, 85%, 90%, 95%, or 100% sequence identity to SEQ ID NOs: 69-73.

45. The anti-CLDN18.2 antibody of claim 43, wherein the humanized antibody or binding fragment thereof comprises a VH region comprising at least 80%, 85%, 90%, 95%, or 100% sequence identity to SEQ ID NOs: 74-76 and a VL region comprising at least 80%, 85%, 90%, 95%, or 100% sequence identity to SEQ ID NOs: 77-80.

46. The anti-CLDN18.2 antibody of claim 43, wherein the humanized antibody or binding fragment thereof comprises a VH region comprising at least 80%, 85%, 90%, 95%, or 100% sequence identity to SEQ ID NOs: 81-84 and a VL region comprising at least 80%, 85%, 90%, 95%, or 100% sequence identity to SEQ ID NOs: 85-88.

47. The anti-CLDN18.2 antibody of claim 43, wherein the humanized antibody or binding fragment thereof comprises a VH region comprising at least 80%, 85%, 90%, 95%, or 100% sequence identity to SEQ ID NOs: 89-92 and a VL region comprising at least 80%, 85%, 90%, 95%, or 100% sequence identity to SEQ ID NOs: 93-97.

48. The anti-CLDN18.2 antibody of any one of the claims 1-47, wherein the anti-CLDN18.2 antibody comprises an IgM framework.

49. The anti-CLDN18.2 antibody of any one of the claims 1-47, wherein the anti-CLDN18.2 antibody comprises an IgG2 framework.

50. The anti-CLDN18.2 antibody of any one of the claims 1-47, wherein the anti-CLDN18.2 antibody comprises an IgG1 framework.

51. The anti-CLDN18.2 antibody of any one of the claims 1-50, wherein the anti-CLDN18.2 antibody comprises one or more mutations in the FC region.

52. The anti-CLDN18.2 antibody of claim 51, wherein the one or more mutations comprise a mutation at amino acid position S239, amino acid position I332, amino acid position F243, amino acid position R292, amino acid position Y300, amino acid position V305, amino acid position P396 or a combination thereof.

53. The anti-CLDN18.2 antibody of claim 51 or 52, wherein one or more mutations in the FC region confer enhanced ADCC to the reference antibody 175D10.

54. The anti-CLDN18.2 antibody of any one of the claims 1-53, wherein the anti-CLDN18.2 antibody has a complement-dependent cytotoxicity (CDC) activity compared to the reference antibody 175D10.

55. The anti-CLDN18.2 antibody of any one of the claims 1-54, wherein the anti-CLDN18.2 antibody is further conjugated to a payload.

56. The anti-CLDN18.2 antibody of claim 55, wherein the payload is an auristatin or its derivative thereof.

57. The anti-CLDN18.2 antibody of claim 56, wherein the auristatin derivative is monomethyl auristatin E (MMAE).

58. The anti-CLDN18.2 antibody of claim 56, wherein the auristatin derivative is monomethyl auristatin F (MMAF).

59. The anti-CLDN18.2 antibody of any one of the claims 55-58, wherein the drug-to-antibody ratio (DAR) is about 2, about 3, or about 4.

60. The anti-CLDN18.2 antibody of any one of the claims 1-59, wherein the anti-CLDN18.2 antibody shares a binding epitope with the reference antibody 175D10.

61. The anti-CLDN18.2 antibody of any one of the claims 1-60, wherein the anti-CLDN18.2 antibody has a cross-binding activity to mouse and cynomolgus CLDN18.2 protein.

62. An anti-Claudin 18.2 (anti-CLDN18.2) antibody that specifically binds to an isoform of CLDN18.2.

63. The anti-CLDN18.2 antibody of claim 62, wherein the isoform of CLDN18.2 is an isoform expressed in cell line SNU620.

64. A nucleic acid polymer encoding an anti-CLDN18.2 antibody of claims 1-63.

65. A vector comprising a nucleic acid polymer of claim 64.

66. A pharmaceutical composition comprising:
an anti-CLDN18.2 antibody of claims 1-63; and
a pharmaceutically acceptable excipient.

67. The pharmaceutical composition of claim 66, wherein the pharmaceutical composition is formulated for systemic administration.

68. The pharmaceutical composition of claim 66 or 67, wherein the pharmaceutical composition is formulated for parenteral administration.

69. A method of treating a subject having a cancer that is characterized with an overexpression of CLDN18.2 protein, comprising:
administering to the subject an anti-CLDN18.2 antibody of claims 1-63 or a pharmaceutical composition of claims 66-68, thereby treating the cancer in the subject.

70. The method of claim 69, wherein the cancer is a gastrointestinal cancer.

71. The method of claim 70, wherein the gastrointestinal cancer is a gastric cancer.

72. The method of claim 70, wherein the gastrointestinal cancer is a pancreatic cancer.

73. The method of claim 70, wherein the gastrointestinal cancer is an esophageal cancer or cholangiocarcinoma.

74. The method of claim 69, wherein the cancer is lung cancer or ovarian cancer.

75. The method of claim 69, wherein further comprising administering to the subject an additional therapeutic agent.

76. The method of claim 75, wherein the additional therapeutic agent comprises a chemotherapeutic agent, an immunotherapeutic agent, a targeted therapeutic agent, a hormone-based therapeutic agent, a stem-cell based therapeutic agent, or radiation.

77. The method of claim 75 or 76, wherein the additional therapeutic agent and the anti-CLDN18.2 antibody are administered simultaneously.

78. The method of claim 75 or 76, wherein the additional therapeutic agent and the anti-CLDN18.2 antibody are administered sequentially.

79. The method of claim 78, wherein the additional therapeutic agent is administered prior to the anti-CLDN18.2 antibody.

80. The method of claim 78, wherein the additional therapeutic agent is administered after the administration of the anti-CLDN18.2 antibody.

81. The method of any one of the claims 75-80, wherein the additional therapeutic agent and the anti-CLDN18.2 antibody are formulated as separate dosage.

82. A method of inducing cell kill effect, comprising:
contacting a plurality of cells with an anti-CLDN18.2 antibody comprising a payload for a time sufficient to internalize the anti-CLDN18.2 antibody and thereby to induce the cell kill effect.

83. The method of claim 82, wherein the anti-CLDN18.2 antibody comprises an anti-CLDN18.2 antibody of claims 1-54.

84. The method of claim 83, wherein the payload comprises a maytansinoid, an auristatin, a taxoid, a calicheamicins, a duocarmycin, an amatoxin, or a derivative thereof.

85. The method of claim 83, wherein the payload comprises an auristatin or its derivative thereof.

86. The method of claim 85, wherein the payload is monomethyl auristatin E (MMAE).

87. The method of claim 85, wherein the payload is monomethyl auristatin F (MMAF).

88. The method of claim 82, wherein the cell is a cancer cell.

89. The method of claim 88, wherein the cell is from a gastrointestinal cancer.

90. The method of claim 89, wherein the gastrointestinal cancer is a gastric cancer.

91. The method of claim 89, wherein the gastrointestinal cancer is a pancreatic cancer.

92. The method of claim 89, wherein the gastrointestinal cancer is an esophageal cancer or cholangiocarcinoma.

93. The method of claim 88, wherein the cell is from a lung cancer or an ovarian cancer.

94. The method of any one of the claims 82-93, wherein the method is an in vitro method.

95. The method of any one of the claims 82-93, wherein the method is an in vivo method.

96. The method of any of the preceding claims, wherein the subject is a human.

97. A kit comprising an anti-CLDN18.2 antibody of claims 1-63, a vector of claim 65, or a pharmaceutical composition of claims 66-68.


SEQUENCE LISTING (101 sequences)

SEQ ID NO: 1 - protein, 10 aa, Artificial Sequence
  Synthetic
GFSLTSYNVY

SEQ ID NO: 2 - protein, 16 aa, Artificial Sequence
  Synthetic
VIWNTGATRYNSTLKS

SEQ ID NO: 3 - protein, 11 aa, Artificial Sequence
  Synthetic
DSAMPAIPFAY

SEQ ID NO: 4 - protein, 16 aa, Artificial Sequence
  Synthetic
VIWNTGATRYQSTLKS

SEQ ID NO: 5 - protein, 16 aa, Artificial Sequence
  Synthetic
VIWNTGATRYESTLKS

SEQ ID NO: 6 - protein, 16 aa, Artificial Sequence
  Synthetic
VIWNTGATRYNSALKS

SEQ ID NO: 7 - protein, 10 aa, Artificial Sequence
  Synthetic
GFTFSSFGMH

SEQ ID NO: 8 - protein, 17 aa, Artificial Sequence
  Synthetic
YISSGSSPIYYVDKLKG

SEQ ID NO: 9 - protein, 11 aa, Artificial Sequence
  Synthetic
AGYAVRNALDY

SEQ ID NO: 10 - protein, 5 aa, Artificial Sequence
  Synthetic
SFGMH

SEQ ID NO: 11 - protein, 17 aa, Artificial Sequence
  Synthetic
YISSGSGSIYYADTVKG

SEQ ID NO: 12 - protein, 12 aa, Artificial Sequence
  Synthetic
ATSYYYGNALEY

SEQ ID NO: 13 - protein, 10 aa, Artificial Sequence
  Synthetic
GFSLTSYGVH

SEQ ID NO: 14 - protein, 16 aa, Artificial Sequence
  Synthetic
VIWPGGNTNYNSALMS

SEQ ID NO: 15 - protein, 8 aa, Artificial Sequence
  Synthetic
DRRLAMDY

SEQ ID NO: 16 - protein, 16 aa, Artificial Sequence
  Synthetic
VIWPGGNTNYESALMS

SEQ ID NO: 17 - protein, 16 aa, Artificial Sequence
  Synthetic
VIWPGGNTNYNIALMS

SEQ ID NO: 18 - protein, 17 aa, Artificial Sequence
  Synthetic
KSSQSLFGSVRQKNYLA

SEQ ID NO: 19 - protein, 7 aa, Artificial Sequence
  Synthetic
LASTRES

SEQ ID NO: 20 - protein, 9 aa, Artificial Sequence
  Synthetic
QQYYDIPWT

SEQ ID NO: 21 - protein, 17 aa, Artificial Sequence
  Synthetic
KSSQSLLNSGNQKNYLA

SEQ ID NO: 22 - protein, 7 aa, Artificial Sequence
  Synthetic
GASTRES

SEQ ID NO: 23 - protein, 9 aa, Artificial Sequence
  Synthetic
QNDLFYPLT

SEQ ID NO: 24 - protein, 17 aa, Artificial Sequence
  Synthetic
KSSQSLLDSGNQKNYLA

SEQ ID NO: 25 - protein, 17 aa, Artificial Sequence
  Synthetic
KSSQSLLESGNQKNYLA

SEQ ID NO: 26 - protein, 17 aa, Artificial Sequence
  Synthetic
KSSQSLLNLGNQKNYLA

SEQ ID NO: 27 - protein, 17 aa, Artificial Sequence
  Synthetic
KSSQSLLNVGNQKNYLA

SEQ ID NO: 28 - protein, 17 aa, Artificial Sequence
  Synthetic
KSSQSLFNSGNQKNYLT

SEQ ID NO: 29 - protein, 7 aa, Artificial Sequence
  Synthetic
WASTRKS

SEQ ID NO: 30 - protein, 9 aa, Artificial Sequence
  Synthetic
QNVYSYPLT

SEQ ID NO: 31 - protein, 17 aa, Artificial Sequence
  Synthetic
KSSQSLFDSGNQKNYLT

SEQ ID NO: 32 - protein, 17 aa, Artificial Sequence
  Synthetic
KSSQSLFESGNQKNYLT

SEQ ID NO: 33 - protein, 17 aa, Artificial Sequence
  Synthetic
KSSQSLFNLGNQKNYLT

SEQ ID NO: 34 - protein, 17 aa, Artificial Sequence
  Synthetic
KSSQSLFNVGNQKNYLT

SEQ ID NO: 35 - protein, 17 aa, Artificial Sequence
  Synthetic
KSSQSLLNSGNQKNYLT

SEQ ID NO: 36 - protein, 7 aa, Artificial Sequence
  Synthetic
WASTRES

SEQ ID NO: 37 - protein, 9 aa, Artificial Sequence
  Synthetic
QNDYSYPFT

SEQ ID NO: 38 - protein, 17 aa, Artificial Sequence
  Synthetic
KSSQSLLESGNQKNYLT

SEQ ID NO: 39 - protein, 17 aa, Artificial Sequence
  Synthetic
KSSQSLLNIGNQKNYLT

SEQ ID NO: 40 - protein, 119 aa, Artificial Sequence
  Synthetic
QVQLKESGPVLVQPSQTLSLTCTVAGFSLTSYNVYWVRQPPGKGLEWMGVIWNTGATRYNSTLKSRLSISKDTSKSQVFLKMNSLQTEDTATYYCARDSAMPAIPFAYWGQGTLVTVSS

SEQ ID NO: 41 - protein, 119 aa, Artificial Sequence
  Synthetic
QVQLKESGPVLVQPSQTLSLTCTVAGFSLTSYNVYWVRQPPGKGLEWMGVIWNTGATRYQSTLKSRLSISKDTSKSQVFLKMNSLQTEDTATYYCARDSAMPAIPFAYWGQGTLVTVSS

SEQ ID NO: 42 - protein, 119 aa, Artificial Sequence
  Synthetic
QVQLKESGPVLVQPSQTLSLTCTVAGFSLTSYNVYWVRQPPGKGLEWMGVIWNTGATRYESTLKSRLSISKDTSKSQVFLKMNSLQTEDTATYYCARDSAMPAIPFAYWGQGTLVTVSS

SEQ ID NO: 43 - protein, 119 aa, Artificial Sequence
  Synthetic
QVQLKESGPVLVQPSQTLSLTCTVAGFSLTSYNVYWVRQPPGKGLEWMGVIWNTGATRYNSALKSRLSISKDTSKSQVFLKMNSLQTEDTATYYCARDSAMPAIPFAYWGQGTLVTVSS

SEQ ID NO: 44 - protein, 113 aa, Artificial Sequence
  Synthetic
DIVMTQSPSSLAVSAGETVTINCKSSQSLFGSVRQKNYLAWYQQKPGQSPKLLIYLASTRESGVPDRFIGSGSGTDFTLTISSVQAEDLAKYYCQQYYDIPWTFGGGTKLELK

SEQ ID NO: 45 - protein, 120 aa, Artificial Sequence
  Synthetic
DVQLVESGGGSVQPGGSRRLSCAASGFTFSSFGMHWVRQAPEKGLEWVAYISSGSSPIYYVDKLKGRFTVSRDNPKNTLFLQMTSLRSEDTAMYYCARAGYAVRNALDYWGQGTSITVSS

SEQ ID NO: 46 - protein, 113 aa, Artificial Sequence
  Synthetic
DIVMTQSPSSLSVSVGEKVTLSCKSSQSLLNSGNQKNYLAWYQQKTGQPPKLLIYGASTRESGVPDRFIGSGSGTDFTLTISSVQAEDLAVYFCQNDLFYPLTFGAGTKLELK

SEQ ID NO: 47 - protein, 113 aa, Artificial Sequence
  Synthetic
DIVMTQSPSSLSVSVGEKVTLSCKSSQSLLDSGNQKNYLAWYQQKTGQPPKLLIYGASTRESGVPDRFIGSGSGTDFTLTISSVQAEDLAVYFCQNDLFYPLTFGAGTKLELK

SEQ ID NO: 48 - protein, 113 aa, Artificial Sequence
  Synthetic
DIVMTQSPSSLSVSVGEKVTLSCKSSQSLLESGNQKNYLAWYQQKTGQPPKLLIYGASTRESGVPDRFIGSGSGTDFTLTISSVQAEDLAVYFCQNDLFYPLTFGAGTKLELK

SEQ ID NO: 49 - protein, 113 aa, Artificial Sequence
  Synthetic
DIVMTQSPSSLSVSVGEKVTLSCKSSQSLLNLGNQKNYLAWYQQKTGQPPKLLIYGASTRESGVPDRFIGSGSGTDFTLTISSVQAEDLAVYFCQNDLFYPLTFGAGTKLELK

SEQ ID NO: 50 - protein, 113 aa, Artificial Sequence
  Synthetic
DIVMTQSPSSLSVSVGEKVTLSCKSSQSLLNVGNQKNYLAWYQQKTGQPPKLLIYGASTRESGVPDRFIGSGSGTDFTLTISSVQAEDLAVYFCQNDLFYPLTFGAGTKLELK

SEQ ID NO: 51 - protein, 119 aa, Artificial Sequence
  Synthetic
DVQLVESGGGLVQPGGSRKLSCAASGFTFSSFGMHWVRQAPEKGLEWVAYISSGSGSIYYADTVKGRFTLSRDNPKNTLFLQMTSLRSEDTAIYYCATSYYYGNALEYWGQGTSVTVSS

SEQ ID NO: 52 - protein, 113 aa, Artificial Sequence
  Synthetic
DVVLTQSPSSLTVTEGEKVSMSCKSSQSLFNSGNQKNYLTWYQQKPGQTPTLLIYWASTRKSGVPDRFTGSGSGTDFTLTINTVQAEDLAVYYCQNVYSYPLTFGAGTKLDLK

SEQ ID NO: 53 - protein, 113 aa, Artificial Sequence
  Synthetic
DVVLTQSPSSLTVTEGEKVSMSCKSSQSLFDSGNQKNYLTWYQQKPGQTPTLLIYWASTRKSGVPDRFTGSGSGTDFTLTINTVQAEDLAVYYCQNVYSYPLTFGAGTKLDLK

SEQ ID NO: 54 - protein, 113 aa, Artificial Sequence
  Synthetic
DVVLTQSPSSLTVTEGEKVSMSCKSSQSLFESGNQKNYLTWYQQKPGQTPTLLIYWASTRKSGVPDRFTGSGSGTDFTLTINTVQAEDLAVYYCQNVYSYPLTFGAGTKLDLK

SEQ ID NO: 55 - protein, 113 aa, Artificial Sequence
  Synthetic
DVVLTQSPSSLTVTEGEKVSMSCKSSQSLFNLGNQKNYLTWYQQKPGQTPTLLIYWASTRKSGVPDRFTGSGSGTDFTLTINTVQAEDLAVYYCQNVYSYPLTFGAGTKLDLK

SEQ ID NO: 56 - protein, 113 aa, Artificial Sequence
  Synthetic
DVVLTQSPSSLTVTEGEKVSMSCKSSQSLFNVGNQKNYLTWYQQKPGQTPTLLIYWASTRKSGVPDRFTGSGSGTDFTLTINTVQAEDLAVYYCQNVYSYPLTFGAGTKLDLK

SEQ ID NO: 57 - protein, 116 aa, Artificial Sequence
  Synthetic
QVQLKESGPGLVAPSQSLSITCTVSGFSLTSYGVHWVRQPPGKGLEWLGVIWPGGNTNYNSALMSRLSISKDNSKSQVFLKMNSLQTDDTAMYYCARDRRLAMDYWGQGTSVTVSS

SEQ ID NO: 58 - protein, 116 aa, Artificial Sequence
  Synthetic
QVQLKESGPGLVAPSQSLSITCTVSGFSLTSYGVHWVRQPPGKGLEWLGVIWPGGNTNYESALMSRLSISKDNSKSQVFLKMNSLQTDDTAMYYCARDRRLAMDYWGQGTSVTVSS

SEQ ID NO: 59 - protein, 116 aa, Artificial Sequence
  Synthetic
QVQLKESGPGLVAPSQSLSITCTVSGFSLTSYGVHWVRQPPGKGLEWLGVIWPGGNTNYNIALMSRLSISKDNSKSQVFLKMNSLQTDDTAMYYCARDRRLAMDYWGQGTSVTVSS

SEQ ID NO: 60 - protein, 113 aa, Artificial Sequence
  Synthetic
DIVMTQSPSSLTVTAGEKVTMTCKSSQSLLNSGNQKNYLTWYQQKPGQPPKLLIYWASTRESGVPDRFTGSGSGTDFTLTISILQAEDLAVYYCQNDYSYPFTFGSGTKLEIK

SEQ ID NO: 61 - protein, 113 aa, Artificial Sequence
  Synthetic
DIVMTQSPSSLTVTAGEKVTMTCKSSQSLLESGNQKNYLTWYQQKPGQPPKLLIYWASTRESGVPDRFTGSGSGTDFTLTISILQAEDLAVYYCQNDYSYPFTFGSGTKLEIK

SEQ ID NO: 62 - protein, 113 aa, Artificial Sequence
  Synthetic
DIVMTQSPSSLTVTAGEKVTMTCKSSQSLLNIGNQKNYLTWYQQKPGQPPKLLIYWASTRESGVPDRFTGSGSGTDFTLTISILQAEDLAVYYCQNDYSYPFTFGSGTKLEIK

SEQ ID NO: 63 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 107 aa, Homo sapiens [sequence omitted]

SEQ ID NO: 65 - protein, 119 aa, Artificial Sequence
  Synthetic
QVQLQESGPGLVKPSETLSLTCTVSGFSLTSYNVYWIRQPPGKGLEWIGVIWNTGATRYNSALKSRVTISVDTSKNQFSLKLSSVTAADTAVYYCARDSAMPAIPFAYWGQGTLVTVSS

SEQ ID NO: 66 - protein, 119 aa, Artificial Sequence
  Synthetic
QVQLQESGPGLVKPSETLSLTCTVSGFSLTSYNVYWIRQPPGKGLEWIGVIWNTGATRYNSALKSRVTISKDTSKNQVSLKLSSVTAADTAVYYCARDSAMPAIPFAYWGQGTLVTVSS

SEQ ID NO: 67 - protein, 119 aa, Artificial Sequence
  Synthetic
QVQLQESGPGLVKPSETLSLTCTVSGFSLTSYNVYWIRQPPGKGLEWIGVIWNTGATRYNSALKSRVTISKDTSKSQVSLKLSSVTAADTAVYYCARDSAMPAIPFAYWGQGTLVTVSS

SEQ ID NO: 68 - protein, 119 aa, Artificial Sequence
  Synthetic
QVQLQESGPGLVKPSETLSLTCTVAGFSLTSYNVYWIRQPPGKGLEWIGVIWNTGATRYNSALKSRVTISKDTSKSQVSLKLSSVTAADTAVYYCARDSAMPAIPFAYWGQGTLVTVSS

SEQ ID NO: 69 - protein, 113 aa, Artificial Sequence
  Synthetic
DIVMTQSPDSLAVSLGERATINCKSSQSLFGSVRQKNYLAWYQQKPGQPPKLLIYLASTRESGVPDRFSGSGSGTDFTLTISSLQAEDVAVYYCQQYYDIPWTFGGGTKVEIK

SEQ ID NO: 70 - protein, 113 aa, Artificial Sequence
  Synthetic
DIVMTQSPDSLAVSLGERATINCKSSQSLFGSVRQKNYLAWYQQKPGQSPKLLIYLASTRESGVPDRFSGSGSGTDFTLTISSLQAEDVAKYYCQQYYDIPWTFGGGTKVEIK

SEQ ID NO: 71 - protein, 113 aa, Artificial Sequence
  Synthetic
DIQMTQSPSSLSASVGDRVTITCKSSQSLFGSVRQKNYLAWYQQKPGKAPKLLIYLASTRESGVPSRFSGSGSGTDFTLTISSLQPEDFATYYCQQYYDIPWTFGGGTKVEIK

SEQ ID NO: 72 - protein, 113 aa, Artificial Sequence
  Synthetic
DIQMTQSPSSLSASVGDRVTITCKSSQSLFGSVRQKNYLAWYQQKPGKSPKLLIYLASTRESGVPDRFSGSGSGTDFTLTISSLQPEDFAKYYCQQYYDIPWTFGGGTKVEIK

SEQ ID NO: 73 - protein, 113 aa, Artificial Sequence
  Synthetic
DIVMTQSPSSLSASVGDRVTINCKSSQSLFGSVRQKNYLAWYQQKPGKSPKLLIYLASTRESGVPDRFSGSGSGTDFTLTISSLQPEDFAKYYCQQYYDIPWTFGGGTKVEIK

SEQ ID NO: 74 - protein, 120 aa, Artificial Sequence
  Synthetic
EVQLVESGGGLVQPGGSLRLSCAASGFTFSSFGMHWVRQAPGKGLEWVSYISSGSSPIYYVDKLKGRFTISRDNAKNSLYLQMNSLRAEDTAVYYCARAGYAVRNALDYWGQGTLVTVSS

SEQ ID NO: 75 - protein, 120 aa, Artificial Sequence
  Synthetic
DVQLVESGGGLVQPGGSLRLSCAASGFTFSSFGMHWVRQAPGKGLEWVAYISSGSSPIYYVDKLKGRFTISRDNAKNSLYLQMNSLRAEDTAVYYCARAGYAVRNALDYWGQGTLVTVSS

SEQ ID NO: 76 - protein, 120 aa, Artificial Sequence
  Synthetic
DVQLVESGGGLVQPGGSLRLSCAASGFTFSSFGMHWVRQAPGKGLEWVAYISSGSSPIYYVDKLKGRFTVSRDNAKNSLYLQMNSLRAEDTAVYYCARAGYAVRNALDYWGQGTLVTVSS

SEQ ID NO: 77 - protein, 113 aa, Artificial Sequence
  Synthetic
EIVMTQSPPTLSLSPGERVTLSCKSSQSLLNVGNQKNYLAWYQQKPGQAPRLLIYGASTRESGIPARFSGSGSGTDFTLTISSLQPEDFAVYYCQNDLFYPLTFGGGTKVEIK

SEQ ID NO: 78 - protein, 113 aa, Artificial Sequence
  Synthetic
DIVMTQSPPTLSLSPGERVTLSCKSSQSLLNVGNQKNYLAWYQQKPGQAPRLLIYGASTRESGIPDRFSGSGSGTDFTLTISSLQPEDFAVYYCQNDLFYPLTFGGGTKVEIK

SEQ ID NO: 79 - protein, 113 aa, Artificial Sequence
  Synthetic
DIVMTQSPPTLSLSPGERVTLSCKSSQSLLNVGNQKNYLAWYQQKPGQAPKLLIYGASTRESGIPDRFSGSGSGTDFTLTISSLQPEDFAVYYCQNDLFYPLTFGGGTKVEIK

SEQ ID NO: 80 - protein, 113 aa, Artificial Sequence
  Synthetic
DIVMTQSPPTLSLSPGERVTLSCKSSQSLLNVGNQKNYLAWYQQKPGQAPKLLIYGASTRESGIPDRFSGSGSGTDFTLTISSLQPEDFAVYFCQNDLFYPLTFGGGTKVEIK

SEQ ID NO: 81 - protein, 120 aa, Artificial Sequence; identical to SEQ ID NO: 74
  Synthetic

SEQ ID NO: 82 - protein, 120 aa, Artificial Sequence
  Synthetic
EVQLVESGGGLVQPGGSLRLSCAASGFTFSSFGMHWVRQAPGKGLEWVAYISSGSSPIYYVDKLKGRFTISRDNAKNSLYLQMNSLRAEDTAVYYCARAGYAVRNALDYWGQGTLVTVSS

SEQ ID NO: 83 - protein, 120 aa, Artificial Sequence
  Synthetic
EVQLVESGGGLVQPGGSLRLSCAASGFTFSSFGMHWVRQAPGKGLEWVAYISSGSSPIYYVDKLKGRFTVSRDNAKNSLYLQMNSLRAEDTAVYYCARAGYAVRNALDYWGQGTLVTVSS

SEQ ID NO: 84 - protein, 120 aa, Artificial Sequence
  Synthetic
EVQLVESGGGLVQPGGSLRLSCAASGFTFSSFGMHWVRQAPGKGLEWVAYISSGSSPIYYVDKLKGRFTVSRDNAKNSLYLQMTSLRAEDTAVYYCARAGYAVRNALDYWGQGTLVTVSS

SEQ ID NO: 85 - protein, 113 aa, Artificial Sequence
  Synthetic
DIVMTQSPDSLAVSLGERATINCKSSQSLLNVGNQKNYLAWYQQKPGQPPKLLIYGASTRESGVPDRFSGSGSGTDFTLTISSLQAEDVAVYYCQNDLFYPLTFGGGTKVEIK

SEQ ID NO: 86 - protein, 113 aa, Artificial Sequence
  Synthetic
DIVMTQSPDSLAVSLGERATINCKSSQSLLNVGNQKNYLAWYQQKPGQPPKLLIYGASTRESGVPDRFSGSGSGTDFTLTISSLQAEDVAVYFCQNDLFYPLTFGGGTKVEIK

SEQ ID NO: 87 - protein, 113 aa, Artificial Sequence
  Synthetic
DIVMTQSPDSLAVSLGERATINCKSSQSLLNVGNQKNYLAWYQQKPGQPPKLLIYGASTRESGVPDRFIGSGSGTDFTLTISSLQAEDVAVYFCQNDLFYPLTFGGGTKVEIK

SEQ ID NO: 88 - protein, 113 aa, Artificial Sequence
  Synthetic
DIVMTQSPDSLAVSLGERATISCKSSQSLLNVGNQKNYLAWYQQKPGQPPKLLIYGASTRESGVPDRFIGSGSGTDFTLTISSLQAEDVAVYFCQNDLFYPLTFGAGTKVEIK

SEQ ID NO: 89 - protein, 119 aa, Artificial Sequence
  Synthetic
EVQLVESGGGLVQPGGSLRLSCAASGFTFSSFGMHWVRQAPGKGLEWVSYISSGSGSIYYADTVKGRFTISRDNAKNSLYLQMNSLRAEDTAVYYCATSYYYGNALEYWGQGTTVTVSS

SEQ ID NO: 90 - protein, 119 aa, Artificial Sequence
  Synthetic
EVQLVESGGGLVQPGGSLRLSCAASGFTFSSFGMHWVRQAPGKGLEWVAYISSGSGSIYYADTVKGRFTISRDNPKNSLYLQMNSLRAEDTAVYYCATSYYYGNALEYWGQGTTVTVSS

SEQ ID NO: 91 - protein, 119 aa, Artificial Sequence
  Synthetic
EVQLVESGGGLVQPGGSLRLSCAASGFTFSSFGMHWVRQAPGKGLEWVAYISSGSGSIYYADTVKGRFTLSRDNPKNSLYLQMNSLRAEDTAVYYCATSYYYGNALEYWGQGTTVTVSS

SEQ ID NO: 92 - protein, 119 aa, Artificial Sequence
  Synthetic
DVQLVESGGGLVQPGGSLRLSCAASGFTFSSFGMHWVRQAPGKGLEWVAYISSGSGSIYYADTVKGRFTLSRDNPKNTLYLQMNSLRAEDTAVYYCATSYYYGNALEYWGQGTTVTVSS

SEQ ID NO: 93 - protein, 113 aa, Artificial Sequence
  Synthetic
DIVMTQSPDSLAVSLGERATINCKSSQSLFNVGNQKNYLTWYQQKPGQPPKLLIYWASTRKSGVPDRFSGSGSGTDFTLTISSLQAEDVAVYYCQNVYSYPLTFGGGTKVEIK

SEQ ID NO: 94 - protein, 113 aa, Artificial Sequence
  Synthetic
DIVLTQSPDSLAVSLGERATINCKSSQSLFNVGNQKNYLTWYQQKPGQTPKLLIYWASTRKSGVPDRFSGSGSGTDFTLTISSLQAEDVAVYYCQNVYSYPLTFGGGTKVEIK

SEQ ID NO: 95 - protein, 113 aa, Artificial Sequence
  Synthetic
DVVLTQSPDSLAVSLGERATINCKSSQSLFNVGNQKNYLTWYQQKPGQTPKLLIYWASTRKSGVPDRFSGSGSGTDFTLTISSLQAEDVAVYYCQNVYSYPLTFGGGTKVEIK

SEQ ID NO: 96 - protein, 113 aa, Artificial Sequence
  Synthetic
DVVLTQSPDSLAVSLGERATISCKSSQSLFNVGNQKNYLTWYQQKPGQTPTLLIYWASTRKSGVPDRFSGSGSGTDFTLTISSLQAEDVAVYYCQNVYSYPLTFGGGTKVEIK

SEQ ID NO: 97 - protein, 113 aa, Artificial Sequence
  Synthetic
DVVLTQSPDSLAVSLGERATISCKSSQSLFNVGNQKNYLTWYQQKPGQTPTLLIYWASTRKSGVPDRFTGSGSGTDFTLTISSLQAEDVAVYYCQNVYSYPLTFGAGTKVEIK

SEQ ID NO: 98 - protein, 467 aa, Artificial Sequence
  Synthetic
MGWSCIILFLVATATGVHSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 240 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - DNA, 786 bp, Homo sapiens
atgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaa

SEQ ID NO: 101 - DNA, 390 bp, Artificial Sequence
  Synthetic
atggagacagacacactcctgctatgggtactgctgctctgggttccaggttccactggtgacgcggcccagccggccaggcgcgcgcgccgtacgaagcttggtaccgagctcggatccactccagtgtggtggaattctgcagatggccgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagcggccatccagcacagtggcggccgctcgaggagggcccgaacaaaaactcatctcagaagaggatctgaatag